SEQUENCE LISTING (146 sequences)

SEQ ID NO: 1 - protein, 976 aa, Homo sapiens [sequence omitted]

SEQ ID NO: 2 - protein, 117 aa, Mus musculus
QVQLQQPGAELVKPGASVKMSCKASGYTFTSYNMHWVKQTPGQGLEWIGVIYSGNGDTSYNQKFKGKATLTADKSSSTAYMQINSLTSEDSAVYYCARERDTRFGNWGQGTLVTVSA

SEQ ID NO: 3 - protein, 111 aa, Mus musculus
NIVLTQSPASLAVSLGLRATISCRASESVDIYGNSFMHWYQQKPGQPPKLLIYLASNLESGVPARFSGSGSRTDFTLTIDPVEADDAATYYCQQNNEDPYTFGGGTKLEIK

SEQ ID NO: 4 - protein, 5 aa, Mus musculus
SYNMH

SEQ ID NO: 5 - protein, 17 aa, Mus musculus
VIYSGNGDTSYNQKFKG

SEQ ID NO: 6 - protein, 8 aa, Mus musculus
ERDTRFGN

SEQ ID NO: 7 - protein, 15 aa, Mus musculus
RASESVDIYGNSFMH

SEQ ID NO: 8 - protein, 7 aa, Mus musculus
LASNLES

SEQ ID NO: 9 - protein, 9 aa, Mus musculus
QQNNEDPYT

SEQ ID NO: 10 - protein, 8 aa, Mus musculus
GYTFTSYN

SEQ ID NO: 11 - protein, 8 aa, Mus musculus
IYSGNGDT

SEQ ID NO: 12 - protein, 10 aa, Mus musculus
ARERDTRFGN

SEQ ID NO: 13 - protein, 10 aa, Mus musculus
ESVDIYGNSF

SEQ ID NO: 14 - skipped

SEQ ID NO: 15 - protein, 10 aa, Mus musculus
QQNNEDPYTF

SEQ ID NO: 16 - protein, 98 aa, Homo sapiens
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCAR

SEQ ID NO: 17 - protein, 120 aa, Homo sapiens
QVQLVQSGAEVKKPGASVKVSCKASGYTFTTYGISWVRQAPGQGLEWMGWISTYNGDTNYAQKLRGRVTMTTDTSTSTAYMELRSLTSDDTAVYYCARDRLSTGTTFYYWGQGTLVTVSS

SEQ ID NO: 18 - protein, 120 aa, Homo sapiens
QVQLVESGAEVKKPGASVKLSCKASGYTFSSYWMHWVRQAPGQRLEWMGEINPDNGHTNYNEKFKSRVTITVDKSASTAYMELSSLRSEDTAVYYCAREADYSYGAFDIWGPGTTVTVSS

SEQ ID NO: 19 - protein, 118 aa, Homo sapiens
QVQLVQSGAEVRKPGASVKLSCKASGYTFTRYSMHWVRQAPGQGLEWMGILNPSGGSTSYAQNFQGRVTLTRDTSTSTVYMELSSLRSEDTAVYYCTRDRLRNWFDPWGQGTLVTVSS

SEQ ID NO: 20 - protein, 98 aa, Homo sapiens
EVQLLESGGGLVQPGGSLRLSCAASGFTFSSYAMSWVRQAPGKGLEWVSVIYSGGSSTYYADSVKGRFTISRDNSKNTLYLQMNSLRAEDTAVYYCAK

SEQ ID NO: 21 - protein, 98 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCAR

SEQ ID NO: 22 - protein, 117 aa, synthetic construct
QVQLVQSGAEVKKPGASVKVSCKASGYTFTSYNMHWVRQAPGQGLEWMGVIYSGNGDTSYNQKFKGRVTMTTDTSTSTAYMELRSLTSDDTAVYYCARERDTRFGNWGQGTLVTVSA

SEQ ID NO: 23 - protein, 117 aa, synthetic construct
QVQLVESGAEVKKPGASVKLSCKASGYTFTSYNMHWVRQAPGQRLEWMGVIYSGNGDTSYNQKFKGRVTITVDKSASTAYMELSSLRSEDTAVYYCARERDTRFGNWGQGTLVTVSA

SEQ ID NO: 24 - protein, 117 aa, synthetic construct
QVQLVQSGAEVRKPGASVKLSCKASGYTFTSYNMHWVRQAPGQGLEWMGVIYSGNGDTSYNQKFKGRVTLTRDTSTSTVYMELSSLRSEDTAVYYCARERDTRFGNWGQGTLVTVSA

SEQ ID NO: 25 - protein, 117 aa, synthetic construct
EVQLLESGGGLVKPGASLRLSCAASGYTFTSYNIHWVRQAPGQGLEWVSAIYSGNVDTSYSQKFKGRFTISRDNSKNTLYLQMNSLRAEDTAVYYCARERDTRFGNWGQGTLVTVSA

SEQ ID NO: 26 - protein, 117 aa, synthetic construct
EVQLVQSGAEVKKPGESLKISCKGSGYTFTSYNMHWVRQMPGKGLEWMGVIYSGNADTSYNQKFKGQVTISADKSISTAYLQWSSLKASDTAMYYCARERDTRFGNWGQGTLVTVSA

SEQ ID NO: 27 - protein, 5 aa, synthetic construct
SYNIH

SEQ ID NO: 28 - protein, 17 aa, synthetic construct
AIYSGNVDTSYSQKFKG

SEQ ID NO: 29 - protein, 17 aa, synthetic construct
VIYSGNADTSYNQKFKG

SEQ ID NO: 30 - protein, 8 aa, synthetic construct
IYSGNVDT

SEQ ID NO: 31 - protein, 8 aa, synthetic construct
IYSGNADT

SEQ ID NO: 32 - protein, 95 aa, Homo sapiens
EIVLTQSPATLSLSPGERATLSCRASQSVSSYLAWYQQKPGQAPRLLIYDASNRATGIPARFSGSGPGTDFTLTISSLEPEDFAVYYCQQRSNWH

SEQ ID NO: 33 - protein, 108 aa, Homo sapiens
EIVLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIKS

SEQ ID NO: 34 - protein, 107 aa, Homo sapiens
DIVMTQSPSTLSASVGDRVTITCRASQSISSWLAWYQQKPGKAPKLLIYEASSLESGVPSRFSGSGSGTEFTLTISSLQPDDFATYYCQQYNSYPYTFGQGTKLEIK

SEQ ID NO: 35 - protein, 95 aa, Homo sapiens
EIVMTQSPATLSVSPGERATLSCRASQSVSSNLAWYQQKPGQAPRLLIYGASTRATGIPARFSGSGSGTEFTLTISSLQSEDFAVYYCQQYNNWP

SEQ ID NO: 36 - protein, 95 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 37 - protein, 95 aa, Homo sapiens
EIVLTQSPATLSLSPGERATLSCRASQGVSSYLAWYQQKPGQAPRLLIYDASNRATGIPARFSGSGPGTDFTLTISSLEPEDFAVYYCQQRSNWH

SEQ ID NO: 38 - protein, 111 aa, synthetic construct
EIVLTQSPSSLSASVGDRVTITCRASESVDIYGNSFMHWYQQKPGKAPKLLIYLASNLESGVPSRFSGSGSGTDFTLTISSLQPEDFATYYCQQNNEDPYTFGQGTKLEIK

SEQ ID NO: 39 - protein, 111 aa, synthetic construct
DIVMTQSPSTLSASVGDRVTITCRASESVDIYGNSFMHWYQQKPGKAPKLLIYLASNLESGVPSRFSGSGSGTEFTLTISSLQPDDFATYYCQQNNEDPYTFGQGTKLEIK

SEQ ID NO: 40 - protein, 111 aa, synthetic construct
DIVMTQSPSTLSASVGDRVTITCRASESVDIYGSSFMHWYQQKPGQPPKLLIYLASNLESGVPSRFSGSGSGTEFTLTISSLEADDVATYYCQQNNEDPYTFGGGTKLEIK

SEQ ID NO: 41 - protein, 111 aa, synthetic construct
EIVMTQSPATLSVSPGERATLSCRASESVDIYGQSFMHWYQQKPGQAPRLLIYLASNLESGIPARFSGSGSGTEFTLTISSLQSEDFAVYYCQQNDEEPYTFGGGTKLEIK

SEQ ID NO: 42 - protein, 111 aa, synthetic construct
DIQMTQSPSSLSASVGDRVTITCRASESVDIYGNAFMHWYQQKPGKAPKLLIYLASNLESGVPSRFSGSGSGTDFTLTISSLQPEDFATYYCQQANEEPYTFGGGTKLEIK

SEQ ID NO: 43 - protein, 111 aa, synthetic construct
EIVLTQSPATLSLSPGERATLSCRASESVDIYGQSFMHWYQQKPGQAPRLLIYLASNLESGLPARFSGSGPGTDFTLTISSLEPEDFAVYYCQQNNENPYTFGGGTKLEIK

SEQ ID NO: 44 - protein, 15 aa, synthetic construct
RASESVDIYGSSFMH

SEQ ID NO: 45 - protein, 15 aa, synthetic construct
RASESVDIYGQSFMH

SEQ ID NO: 46 - protein, 15 aa, synthetic construct
RASESVDIYGNAFMH

SEQ ID NO: 47 - protein, 10 aa, synthetic construct
ESVDIYGSSF

SEQ ID NO: 48 - protein, 10 aa, synthetic construct
ESVDIYGQSF

SEQ ID NO: 49 - protein, 10 aa, synthetic construct
ESVDIYGNAF

SEQ ID NO: 50 - protein, 9 aa, synthetic construct
QQNDEEPYT

SEQ ID NO: 51 - protein, 9 aa, synthetic construct
QQANEEPYT

SEQ ID NO: 52 - protein, 9 aa, synthetic construct
QQNNENPYT

SEQ ID NO: 53 - protein, 10 aa, synthetic construct
QQNDEEPYTF

SEQ ID NO: 54 - protein, 10 aa, synthetic construct
QQANEEPYTF

SEQ ID NO: 55 - protein, 10 aa, synthetic construct
QQNNENPYTF

SEQ ID NO: 56 - protein, 114 aa, synthetic construct
EIVLTQSPSSLSASVGDRVTITCRASESVDIYGQSFMHWYQQKPGKAPKLLIYLASNLESGVPSRFSGSGSGTDFTLTISSLQPEDFATYYCQQNNEDPYTFGQGTKLEIKRTV

SEQ ID NO: 57 - protein, 114 aa, synthetic construct
EIVLTQSPSSLSASVGDRVTITCRASESVDIYGRSFMHWYQQKPGKAPKLLIYLASNLESGVPSRFSGSGSGTDFTLTISSLQPEDFATYYCQQNNEDPYTFGQGTKLEIKRTV

SEQ ID NO: 58 - protein, 114 aa, synthetic construct
EIVLTQSPSSLSASVGDRVTITCRASESVDIYGSSFMHWYQQKPGKAPKLLIYLASNLESGVPSRFSGSGSGTDFTLTISSLQPEDFATYYCQQNNEDPYTFGQGTKLEIKRTV

SEQ ID NO: 59 - protein, 114 aa, synthetic construct
EIVLTQSPSSLSASVGDRVTITCRASESVDIYGKSFMHWYQQKPGKAPKLLIYLASNLESGVPSRFSGSGSGTDFTLTISSLQPEDFATYYCQQNNEDPYTFGQGTKLEIKRTV

SEQ ID NO: 60 - protein, 114 aa, synthetic construct
EIVLTQSPSSLSASVGDRVTITCRASESVDIYGFSFMHWYQQKPGKAPKLLIYLASNLESGVPSRFSGSGSGTDFTLTISSLQPEDFATYYCQQNNEDPYTFGQGTKLEIKRTV

SEQ ID NO: 61 - protein, 114 aa, synthetic construct
EIVLTQSPSSLSASVGDRVTITCRASESVDIYGESFMHWYQQKPGKAPKLLIYLASNLESGVPSRFSGSGSGTDFTLTISSLQPEDFATYYCQQNNEDPYTFGQGTKLEIKRTV

SEQ ID NO: 62 - protein, 114 aa, synthetic construct
EIVLTQSPSSLSASVGDRVTITCRASESVDIYGYSFMHWYQQKPGKAPKLLIYLASNLESGVPSRFSGSGSGTDFTLTISSLQPEDFATYYCQQNNEDPYTFGQGTKLEIKRTV

SEQ ID NO: 63 - protein, 114 aa, synthetic construct
EIVLTQSPSSLSASVGDRVTITCRASESVDIYGNSFMHWYQQKPGKAPKLLIYLASNLESGVPSRFSGSGSGTDFTLTISSLQPEDFATYYCQQGNEDPYTFGQGTKLEIKRTV

SEQ ID NO: 64 - protein, 114 aa, synthetic construct
EIVLTQSPSSLSASVGDRVTITCRASESVDIYGNSFMHWYQQKPGKAPKLLIYLASNLESGVPSRFSGSGSGTDFTLTISSLQPEDFATYYCQQRNEDPYTFGQGTKLEIKRTV

SEQ ID NO: 65 - protein, 114 aa, synthetic construct
EIVLTQSPSSLSASVGDRVTITCRASESVDIYGNSFMHWYQQKPGKAPKLLIYLASNLESGVPSRFSGSGSGTDFTLTISSLQPEDFATYYCQQENEDPYTFGQGTKLEIKRTV

SEQ ID NO: 66 - protein, 114 aa, synthetic construct
EIVLTQSPSSLSASVGDRVTITCRASESVDIYGNSFMHWYQQKPGKAPKLLIYLASNLESGVPSRFSGSGSGTDFTLTISSLQPEDFATYYCQQHNEDPYTFGQGTKLEIKRTV

SEQ ID NO: 67 - protein, 114 aa, synthetic construct
EIVLTQSPSSLSASVGDRVTITCRASESVDIYGNSFMHWYQQKPGKAPKLLIYLASNLESGVPSRFSGSGSGTDFTLTISSLQPEDFATYYCQQNKEDPYTFGQGTKLEIKRTV

SEQ ID NO: 68 - protein, 114 aa, synthetic construct
EIVLTQSPSSLSASVGDRVTITCRASESVDIYGNSFMHWYQQKPGKAPKLLIYLASNLESGVPSRFSGSGSGTDFTLTISSLQPEDFATYYCQQNNEPPYTFGQGTKLEIKRTV

SEQ ID NO: 69 - protein, 114 aa, synthetic construct
EIVLTQSPSSLSASVGDRVTITCRASESVDIYGNSFMHWYQQKPGKAPKLLIYLASNLESGVPSRFSGSGSGTDFTLTISSLQPEDFATYYCQQNNEEPYTFGQGTKLEIKRTV

SEQ ID NO: 70 - protein, 117 aa, synthetic construct
QVQLVQSGAEVKKPGASVKVSCKASGYTFTSYNMHWVRQAPGQGLEWMGVIYSGSGDTSYNQKFKGRVTMTTDTSTSTAYMELRSLTSDDTAVYYCARERDTRFGNWGQGTLVTVSA

SEQ ID NO: 71 - protein, 17 aa, synthetic construct
VIYSGSGDTSYNQKFKG

SEQ ID NO: 72 - protein, 8 aa, synthetic construct
IYSGSGDT

SEQ ID NO: 73 - protein, 15 aa, synthetic construct
RASESVDIYGRSFMH

SEQ ID NO: 74 - protein, 10 aa, synthetic construct
ESVDIYGRSF

SEQ ID NO: 75 - protein, 15 aa, synthetic construct
RASESVDIYGKSFMH

SEQ ID NO: 76 - protein, 10 aa, synthetic construct
ESVDIYGKSF

SEQ ID NO: 77 - protein, 15 aa, synthetic construct
RASESVDIYGFSFMH

SEQ ID NO: 78 - protein, 10 aa, synthetic construct
ESVDIYGFSF

SEQ ID NO: 79 - protein, 15 aa, synthetic construct
RASESVDIYGESFMH

SEQ ID NO: 80 - protein, 10 aa, synthetic construct
ESVDIYGESF

SEQ ID NO: 81 - protein, 15 aa, synthetic construct
RASESVDIYGYSFMH

SEQ ID NO: 82 - protein, 10 aa, synthetic construct
ESVDIYGYSF

SEQ ID NO: 83 - protein, 9 aa, synthetic construct
QQGNEDPYT

SEQ ID NO: 84 - protein, 10 aa, synthetic construct
QQGNEDPYTF

SEQ ID NO: 85 - protein, 9 aa, synthetic construct
QQRNEDPYT

SEQ ID NO: 86 - protein, 10 aa, synthetic construct
QQRNEDPYTF

SEQ ID NO: 87 - protein, 9 aa, synthetic construct
QQENEDPYT

SEQ ID NO: 88 - protein, 10 aa, synthetic construct
QQENEDPYTF

SEQ ID NO: 89 - protein, 9 aa, synthetic construct
QQHNEDPYT

SEQ ID NO: 90 - protein, 10 aa, synthetic construct
QQHNEDPYTF

SEQ ID NO: 91 - protein, 9 aa, synthetic construct
QQNKEDPYT

SEQ ID NO: 92 - protein, 10 aa, synthetic construct
QQNKEDPYTF

SEQ ID NO: 93 - protein, 9 aa, synthetic construct
QQNNEPPYT

SEQ ID NO: 94 - protein, 10 aa, synthetic construct
QQNNEPPYTF

SEQ ID NO: 95 - protein, 9 aa, synthetic construct
QQNNEEPYT

SEQ ID NO: 96 - protein, 10 aa, synthetic construct
QQNNEEPYTF

SEQ ID NO: 97 - protein, 114 aa, synthetic construct
EIVLTQSPSSLSASVGDRVTITCRASESVDIYGYSFMHWYQQKPGKAPKLLIYLASNLESGVPSRFSGSGSGTDFTLTISSLQPEDFATYYCQQNNEEPYTFGQGTKLEIKRTV

SEQ ID NO: 98 - protein, 114 aa, synthetic construct
EIVLTQSPSSLSASVGDRVTITCRASESVDIYGESFMHWYQQKPGKAPKLLIYLASNLESGVPSRFSGSGSGTDFTLTISSLQPEDFATYYCQQNNEEPYTFGQGTKLEIKRTV

SEQ ID NO: 99 - protein, 114 aa, synthetic construct
EIVLTQSPSSLSASVGDRVTITCRASESVDIYGYSFMHWYQQKPGKAPKLLIYLASNLESGVPSRFSGSGSGTDFTLTISSLQPEDFATYYCQQNKEEPYTFGQGTKLEIKRTV

SEQ ID NO: 100 - protein, 114 aa, synthetic construct
EIVLTQSPSSLSASVGDRVTITCRASESVDIYGESFMHWYQQKPGKAPKLLIYLASNLESGVPSRFSGSGSGTDFTLTISSLQPEDFATYYCQQNKEEPYTFGQGTKLEIKRTV

SEQ ID NO: 101 - protein, 114 aa, synthetic construct
EIVLTQSPSSLSASVGDRVTITCRASESVDIYGQSFMHWYQQKPGKAPKLLIYLASNLESGVPSRFSGSGSGTDFTLTISSLQPEDFATYYCQQNKEEPYTFGQGTKLEIKRTV

SEQ ID NO: 102 - protein, 114 aa, synthetic construct
EIVLTQSPSSLSASVGDRVTITCRASESVDIYGSSFMHWYQQKPGKAPKLLIYLASNLESGVPSRFSGSGSGTDFTLTISSLQPEDFATYYCQQNKEEPYTFGQGTKLEIKRTV

SEQ ID NO: 103 - protein, 114 aa, synthetic construct
EIVLTQSPSSLSASVGDRVTITCRASESVDIYGHSFMHWYQQKPGKAPKLLIYLASNLESGVPSRFSGSGSGTDFTLTISSLQPEDFATYYCQQNKEEPYTFGQGTKLEIKRTV

SEQ ID NO: 104 - protein, 114 aa, synthetic construct
EIVLTQSPSSLSASVGDRVTITCRASESVDIYGSSFMHWYQQKPGKAPKLLIYKASNLESGVPSRFSGSGSGTDFTLTISSLQPEDFATYYCQQNKEEPYTFGQGTKLEIKRTV

SEQ ID NO: 105 - protein, 114 aa, synthetic construct
EIVLTQSPSSLSASVGDRVTITCRASESVDIYGESFMHWYQQKPGKAPKLLIYLASNLESGVPSRFSGSGSGTDFTLTISSLQPEDFATYYCQQNREEPYTFGQGTKLEIKRTV

SEQ ID NO: 106 - protein, 117 aa, synthetic construct
QVQLVQSGAEVKKPGASVKVSCKASGYTFTSYNMHWVRQAPGQGLEWMGVIYSGQGDTSYNQKFKGRVTMTTDTSTSTAYMELRSLTSDDTAVYYCARERDTRFGNWGQGTLVTVSA

SEQ ID NO: 107 - protein, 17 aa, synthetic construct
VIYSGQGDTSYNQKFKG

SEQ ID NO: 108 - protein, 8 aa, synthetic construct
IYSGQGDT

SEQ ID NO: 109 - protein, 15 aa, synthetic construct
RASESVDIYGHSFMH

SEQ ID NO: 110 - protein, 10 aa, synthetic construct
ESVDIYGHSF

SEQ ID NO: 111 - protein, 7 aa, synthetic construct
KASNLES

SEQ ID NO: 112 - skipped

SEQ ID NO: 113 - protein, 9 aa, synthetic construct
QQNKEEPYT

SEQ ID NO: 114 - protein, 10 aa, synthetic construct
QQNKEEPYTF

SEQ ID NO: 115 - protein, 9 aa, synthetic construct
QQNREEPYT

SEQ ID NO: 116 - protein, 10 aa, synthetic construct
QQNREEPYTF

SEQ ID NO: 117 - protein, 111 aa, synthetic construct
EIVLTQSPSSLSASVGDRVTITCRASESVDIYGESFMHWYQQKPGKAPKLLIYHASNLESGVPSRFSGSGSGTDFTLTISSLQPEDFATYYCQQNREEPYTFGQGTKLEIK

SEQ ID NO: 118 - protein, 111 aa, synthetic construct
EIVLTQSPSSLSASVGDRVTITCRASESVDIYGESFMHWYQQKPGKAPKLLIYEASNLESGVPSRFSGSGSGTDFTLTISSLQPEDFATYYCQQNREEPYTFGQGTKLEIK

SEQ ID NO: 119 - protein, 111 aa, synthetic construct
EIVLTQSPSSLSASVGDRVTITCRASESVDIYGESFMHWYQQKPGKAPKLLIYSASNLESGVPSRFSGSGSGTDFTLTISSLQPEDFATYYCQQNREEPYTFGQGTKLEIK

SEQ ID NO: 120 - protein, 111 aa, synthetic construct
EIVLTQSPSSLSASVGDRVTITCRASESVDIYGESFMHWYQQKPGKAPKLLIYQASNLESGVPSRFSGSGSGTDFTLTISSLQPEDFATYYCQQNREEPYTFGQGTKLEIK

SEQ ID NO: 121 - protein, 7 aa, synthetic construct
HASNLES

SEQ ID NO: 122 - skipped

SEQ ID NO: 123 - protein, 7 aa, synthetic construct
EASNLES

SEQ ID NO: 124 - skipped

SEQ ID NO: 125 - protein, 7 aa, synthetic construct
SASNLES

SEQ ID NO: 126 - skipped

SEQ ID NO: 127 - protein, 7 aa, synthetic construct
QASNLES

SEQ ID NO: 128 - skipped

SEQ ID NO: 129 - protein, 128 aa, synthetic construct
QVQLQQSGPGLVKPSQTLSLTCAISGDSVSSNSAAWSWIRQSPGRGLEWLGRIYYRSKWYNDYAVSVKSRITINPDTSKNQFSLQLNSVTPEDTAVYYCARLDHRYHEDTVYPGMDVWGQGTLVTVSS

SEQ ID NO: 130 - protein, 107 aa, synthetic construct
DIELTQPPSVSVAPGQTARISCSGDNLPAYTVTWYQQKPGQAPVLVIYDDSDRPSGIPERFSGSNSGNTATLTISGTQAEDEADYYCASWDPSSGVVFGGGTKLTVL

SEQ ID NO: 131 - protein, 7 aa, synthetic construct
SNSAAWS

SEQ ID NO: 132 - protein, 18 aa, synthetic construct
RIYYRSKWYNDYAVSVKS

SEQ ID NO: 133 - protein, 16 aa, synthetic construct
LDHRYHEDTVYPGMDV

SEQ ID NO: 134 - protein, 11 aa, synthetic construct
SGDNLPAYTVT

SEQ ID NO: 135 - protein, 7 aa, synthetic construct
DDSDRPS

SEQ ID NO: 136 - protein, 10 aa, synthetic construct
ASWDPSSGVV

SEQ ID NO: 137 - protein, 10 aa, synthetic construct
GDSVSSNSAA

SEQ ID NO: 138 - protein, 9 aa, synthetic construct
IYYRSKWYN

SEQ ID NO: 139 - protein, 18 aa, synthetic construct
ARLDHRYHEDTVYPGMDV

SEQ ID NO: 140 - protein, 6 aa, synthetic construct
NLPAYT

SEQ ID NO: 141 - skipped

SEQ ID NO: 142 - protein, 117 aa, synthetic construct
QVQLVQSGAEVKKPGASVKVSCKASGYTFTSYNMHWVRQAPGQGLEWMGVIYSGNGDTSYNQKFKGRVTITADKSTSTAYMELSSLRSEDTAVYYCARERDTRFGNWGQGTLVTVSS

SEQ ID NO: 143 - protein, 112 aa, synthetic construct
DIVMTQSPDSLAVSLGERATINCRASESVDIYGNSFMHWYQQKPGQPPKLLIYLASNLESGVPDRFSGSGSGTDFTLTISSLQAEDVAVYYCQQNNEDPYTFGGGTKVEIKR

SEQ ID NO: 144 - protein, 117 aa, synthetic construct
QVQLVQSGAEVKKPGASVKVSCKASGYTFTSYNMHWVRQAPGQGLEWMGVIYSGNGDTSYAQKFQGRVTITADKSTSTAYMELSSLRSEDTAVYYCARERDTRFGNWGQGTLVTVSS

SEQ ID NO: 145 - protein, 111 aa, synthetic construct
DIVMTQSPLSLPVTPGEPASISCRASESVDIYGQSFMHWYLQKPGQSPQLLIYLASNLESGVPDRFSGSGSGTDFTLKISRVEAEDVGVYYCQQNNEDPYTFGGGTKVEIK

SEQ ID NO: 146 - protein, 17 aa, synthetic construct
VIYSGNGDTSYAQKFQG

(19) United States
(12) Patent Application Publication
(10) Pub. No.: US 2026/0258124 A1
(43) Pub. Date: Sep. 3, 2026

(54) ANTIBODIES TARGETING CD117

(71) Applicants: CIMEIO THERAPEUTICS AG, BASEL (CH); UNIVERSITÄT BASEL, BALE (CH)

(72) Inventors: Stefanie URLINGER, MUNCHEN (DE); Anna CAMUS, BASEL (CH); Rosalba LEPORE, BASEL (CH); Alessandro SINOPOLI, SAINT-LOUIS (FR); Amélie WIEDERKEHR, RANSPACH LE BAS (FR); Lukas JEKER, RIEHEN (CH); Frank LEHMANN, BAD SACKINGEN (DE); Anna HAYDN, ZURICH (CH); Mathilde TESTUT, HUNINGUE (FR); Lisa WELLINGER, BASEL (CH); Laura GARCIA PRAT, BASEL (CH); Emiel TEN BUREN, AARBURG (CH); Melanie HUG, HOLSTEIN (CH)

(21) Appl. No.: 18/871,440

(22) PCT Filed: Jul. 7, 2023
(86) PCT No.: PCT/EP2023/068817

§ 371 Date: Dec. 3, 2024

(30) Foreign Application Priority Data
Jul. 7, 2022 (EP) 22183671.1
Nov. 16, 2022 (EP) 22207927.9
Dec. 16, 2022 (EP) 22214286.1

(51) Int. Cl. C07K 16/28

(52) U.S. Cl. CPC C07K 16/2803; C07K 2317/24

(57) ABSTRACT

The present disclosure relates to antibodies and antibody fragments that are specific for CD117. The antibodies are improved versions of state-of-the art antibodies. In addition to a sophisticated humanization campaign, the antibodies were also engineered to remove several detrimental motifs within the CDR region without losing any beneficial properties. The antibodies are useful for the treatment of diseases associated with CD117.

### FIELD OF THE INVENTION

The present disclosure relates to antibodies and antibody fragments that are specific for CD117. The antibodies are improved versions of state-of-the art antibodies. In addition to a sophisticated humanization campaign, the antibodies were also engineered to remove several detrimental motifs within the CDR region without losing any beneficial properties. The antibodies are useful for the treatment of diseases associated with CD117.

### STATEMENT REGARDING FUNDING

The project leading to this application has received funding from the European Research Council (ERC) under the European Union's Horizon 2020 research and innovation programme (grant agreement No 818806).

### BACKGROUND

CD117 (also referred to as c-kit or Stem Cell Factor Receptor (SCRF)) is a single transmembrane, receptor tyrosine kinase that binds the ligand Stem Cell Factor (SCF). SCF induces homodimerization of CD117 which activates its tyrosine kinase activity and results in signaling through both the PI3K-AKT and MAPK pathways (Kindblom et al, Am J. Path. 1998 152(5):1 259).

CD117 was initially discovered as an oncogene and has been studied in the field of oncology (see, for example, Stankov et al. (2014) Curr Pharm Des. 20:2849-80). CD117 is highly expressed on hematopoietic stem cells (HSCs) and its ligand is stem cell factor, SCF. SCF is a growth factor that acts through CD117 and is required for the survival, proliferation, and differentiation of hematopoietic cells. The expression of CD117 on HSCs together with its activity in regulating hematopoiesis make CD117 a potential target for bone marrow conditioning across a broad range of diseases (Russkamp et al. Exp. Hematol. (2021) 95:31-45; Czechowicz et al. Nat Commun (2019) 10: 617). There remains, however, a need for anti-CD117 based therapy that is effective for conditioning a patient for transplantation, such as a bone marrow transplantation.

Several anti-CD117 moieties are known in the art, some of which are currently in development. Antibody SR-1 was originally isolated from a hybridoma (WO1992017505). Blood (2019) 133: 2069-78 describes the use of SR-1 in HSC-driven blood and immune disorders. Certain humanized versions of SR-1 were generated (WO2007127317; WO2020112687). WO2020033664 discloses the original SR-1 antibody (with the sequence of the variable heavy chain and the variable light chains being identical to the sequences disclosed in US20200165337A1) for use in bispecific and multispecific constructs. J. Clin. Oncol. (2021) Vol. 39 No. 15 Meeting Abstract 2021 ASCO Annual Meeting, abstract 7035 discloses results of a clinical Phase I trail of antibody JSP191. Blood (2019) 134 (Suppl. 1):4428 discloses a study relate to the anti-CD117 antibody FSI-174. Anti-CD117 drug conjugates are described in WO2016020791. Other anti-CD117 antibodies are described in WO2015050959 and WO2019084064. Certain anti-CD117 antibodies are also available commercially, e.g. antibody 104D2 Dianova (#117PE-100T). These and other anti-CD117 moieties may be used in the context of the present disclosure. WO2021041945 discloses genomic alterations of antigens, including CD117, generated by base editing. These antigen alterations were however not characterized, in particular the biological function of the variants was not tested.

### SUMMARY OF THE INVENTION

The present disclosure relates to a humanized antibody or antibody fragments specific for human CD117, wherein said antibodies or antibody fragments comprise a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9, wherein the humanized antibody or antibody fragment has at least a mutation in which the asparagine in the LCDR1 region (SEQ ID NO: 7) is replaced by glutamic acid or tyrosine. In certain embodiments said humanized antibody or antibody fragment has one or more of the additional following mutations:
a) the first asparagine in the HCDR2 region (SEQ ID NO: 5) is replaced by a serine,
b) the second asparagine in the LCDR3 region (SEQ ID NO: 9) is replaced by lysine, or
c) the aspartic acid in the LCDR3 region (SEQ ID NO: 9) is replaced by glutamic acid.

The present disclosure relates to a humanized antibody or antibody fragment, wherein said humanized antibody or antibody fragment comprises:
a) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 95,
b) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 81, the LCDR2 region of SEQ ID NO: 8 and the LCDR3 region of SEQ ID NO: 113,
c) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 113,
d) a variable heavy chain comprising a H CDR1 region of SEQ ID NO: 4, a HCDR2 region of SEQ ID NO: 71, a HCDR3 region of SEQ ID NO: 6 and a variable light chain comprising, the LCDR1 region of SEQ ID NO: 79, the LCDR2 region of SEQ ID NO:8 and the LCDR3 region of SEQ ID NO:95,
e) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 81, the LCDR2 region of SEQ ID NO: 8 and the LCDR3 region of SEQ ID NO: 95,
f) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 9, or
g) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 81, the LCDR2 region of SEQ ID NO: 8 and the LCDR3 region of SEQ ID NO: 9.

The present disclosure relates to a humanized antibody or antibody fragment, wherein said humanized antibody or antibody fragment comprises:
a) a variable heavy chain of SEQ ID NO: 22 and a variable light chain of SEQ ID NO: 98,
b) a variable heavy chain of SEQ ID NO: 22 and a variable light chain of SEQ ID NO: 99,
c) a variable heavy chain of SEQ ID NO: 22 and a variable light chain of SEQ ID NO: 100,
d) a variable heavy chain of SEQ ID NO: 70 and a variable light chain of SEQ ID NO: 98,
e) a variable heavy chain of SEQ ID NO: 22 and a variable light chain of SEQ ID NO: 97,
f) a variable heavy chain of SEQ ID NO: 22 and a variable light chain of SEQ ID NO: 61, or
g) a variable heavy chain of SEQ ID NO: 22 and a variable light chain of SEQ ID NO: 62.

The present disclosure also relates to humanized antibody or antibody fragment, wherein said humanized antibody or antibody fragment inhibits SCF binding to CD117, SCF-dependent proliferation of CD117-positive cells and/or SCF-dependent phosphorylation of CD117.

The present disclosure also relates to humanized antibody or antibody fragment, wherein said humanized antibody or antibody fragment binds to CD117 with about the same affinity as an antibody or antibody fragment comprising a variable heavy chain comprising a HCDR1 region of SEQ ID NO: 4, a HCDR2 region of SEQ ID NO: 5, a HCDR3 region of SEQ ID NO: 6 and a variable heavy chain comprising, the LCDR1 region of SEQ ID NO: 7, the LCDR2 region of SEQ ID NO: 8 and the LCDR3 region of SEQ ID NO: 9.

The present disclosure also relates to humanized antibody or antibody fragment, wherein said humanized antibody or antibody fragment binds to CD117 with a higher affinity than an antibody or antibody fragment comprising a variable heavy chain comprising a HCDR1 region of SEQ ID NO: 4, a HCDR2 region of SEQ ID NO: 5, a HCDR3 region of SEQ ID NO: 6 and a variable heavy chain comprising, the LCDR1 region of SEQ ID NO: 7, the LCDR2 region of SEQ ID NO: 8 and the LCDR3 region of SEQ ID NO: 9.

The present disclosure also relates to humanized antibody or antibody fragment, wherein said humanized antibody or antibody fragment binds to the same epitope on CD117 as an antibody or antibody fragment comprising a variable heavy chain comprising a HCDR1 region of SEQ ID NO: 4, a HCDR2 region of SEQ ID NO: 5, a HCDR3 region of SEQ ID NO: 6 and a variable heavy chain comprising, the LCDR1 region of SEQ ID NO: 7, the LCDR2 region of SEQ ID NO: 8 and the LCDR3 region of SEQ ID NO: 9.

The present disclosure also relates to humanized antibody or antibody fragment, wherein said humanized antibody or antibody is cross-reactive with cynomolgus monkey.

The present disclosure also relates to humanized antibody or antibody fragment as disclosed herein for use in medicine, preferably the treatment of cancer, such as a hematological cancer (such as AML) or a solid cancer (such as mast cell cancer, testicular stromal cancer, gastrointestinal stromal cancer, melanoma, breast and lung cancer) or for the conditioning of bone marrow.

The present disclosure also relates to nucleic acids or a nucleic acid composition comprising a nucleic acid sequence or a plurality of nucleic acid sequences encoding the humanized antibody or antibody fragment of the present disclosure.

The present disclosure also relates to vectors comprising nucleic acids or nucleic acid compositions encoding the humanized antibody or antibody fragment of the present disclosure.

The present disclosure also relates to host cells comprising the vectors or the nucleic acids or nucleic acid composition of the humanized antibody or antibody fragment of the present disclosure.

The present disclosure also relates to pharmaceutical compositions comprising the humanized antibody or antibody fragment of the present disclosure and a pharmaceutically acceptable carrier or excipient.

### FIGURE LEGENDS

FIG. 1 shows the alignment of five humanized variants to the original murine VH sequence of SR-1. VHO is the murine sequence and VH1-5 are the humanized variants. CDRs are underlined. Key residues important for the VH/VL interface and canonical loop structure have been maintained as much as possible.

FIG. 2 shows the alignment of five humanized variants to the original murine VL sequence of SR-1. VLO is the murine sequence and VL1-6 are the humanized variants. CDRs are underlined. Key residues important for the VH/VL interface and canonical loop structure have been maintained as much as possible.

FIG. 3 shows the result of an assay measuring the antibody-mediated inhibition of SCF-dependent phosphorylation of CD117. From left to the right the following antibodies were tested: A—NegCtrl, B—SR-1, C—AMG191, D—FSI-174, E—Dufour, F—Matterhorn, G—Piz Bernina, H—Allalinhorn, I—Piz Zupo, J—Eiger, K—Piz Argient, L—Piz Roseg, M—Bietschhorn, N—Bellavista.

FIG. 4 shows the result of an experiment investigating if the antibodies of the present disclosure are able to inhibit binding of CD117 to SCF. Tested were the following antibodies: NegCtrl, SR-1, AMG191, FSI-174, Dufour, Matterhorn, Piz Bernina, Allalinhorn, Piz Zupo, Eiger, Piz Argient, Piz Roseg, Bietschhorn and Bellavista. Except Piz Roseg and Piz Argient, all antibodies of the present disclosure effectively inhibited binding of SCF to CD117. The NegCtrl antibody (anti-chicken lysozyme) did not inhibit binding of SCF to CD117.

FIG. 5 shows the result of an experiment investigating binding of the antibodies of the present disclosure to CD117 on TF-1 cells. Except for Piz Roseg and Piz Argient, all antibodies of the present disclosure strongly bound to CD117 on TF-1 cells. Binding could also be detected for Piz Roseg and Piz Argient, although only at higher antibody concentrations. The NegCtrl antibody (anti-chicken lysozyme) did not show any binding to TF-1 cells.

FIG. 6 shows the result of an experiment investigating if the antibodies of the present disclosure are able to inhibit binding of CD117 to SCF in HSPCs (CD34+CD38− cells) and HSC's (CD34+CD38− CD90+CD45RA− cells) in Low cytokine medium and High cytokine medium. For both tested cell populations and both media the same result was achieved. Antibodies SR-1 and Allalinhorn both inhibited binding of SCF to CD117 completely and to the same extent. The NegCtrl antibody (anti-chicken lysozyme) did not inhibit binding of SCF to CD117.

FIG. 7 shows the result of antibody-mediated inhibition of HSPC colony formation. All three anti-CD117 antibodies tested led to a dose-dependent reduction of colony. Reduction in colony formation was most pronounced with antibody Allalinhorn.

FIG. 8 shows the SCF-dependent phosphorylation of CD117 at position Tyr719 of wild-type TF-1 cells and E73K, variants of CD117. D121K and S123K variants of CD117 behaved similarly (data not shown).

FIG. 9 shows that SCF-dependent CD117 phosphorylation in wild-type TF-1 cells is blocked by antibodies Piz Bernina and Allalinhorn, whereas in TF-1 cells with D121K and S123K variants of CD117 phosphorylation is unaffected. The same is also observed for variants E73K, E73Y and S123F (data not shown).

FIG. 10 shows that antibody Allalinhorn effectively depletes HSPCs in vivo.

FIG. 11 shows that in contrast to antibody Allalinhorn, binding of the isotope control antibody is not affected by E73K.

FIG. 12 shows that wild-type DF-1 cells, but not DF-1 cells transfected with D121K or S123K variants of CD117, are depleted by the tested ADCs (Piz Bernina-teserine and Allalinhorn-teserine). D121K A5 and D121K A6 designate two different batches of plasmids for the same variant.

FIG. 13 shows depletion of unedited CD34+ progenitor cells (gated as live/hCD45+/CD34+/CD38−) in mice receiving antibody Allalinhorn compared to mice receiving an isotype control antibody. In contrast, injection of Allalinhorn resulted in an enrichment of E73K, D121K and S123K-edited variant cells compared to animals receiving isotype control antibody.

### DEFINITIONS

The disclosure pertains to antibodies, which specifically bind to CD117, and uses of such antibodies, in particular therapeutic uses.

The term “CD117” refers to a protein also known as KIT, c-Kit, SCFR, PBT or MASTC. Human CD117 has the following amino acid sequence (UniProt P10721, updated on Jun. 28, 2023):

(SEQ ID NO. 1)
MRGARGAWDFLCVLLLLLRVQTGSSQPSVSPGEPSPPSIHPGKSDLIVRVGDEIRLLCTD
PGFVKWTFEILDETNENKQNEWITEKAEATNTGKYTCTNKHGLSNSIYVFVRDPAKLFLV
DRSLYGKEDNDTLVRCPLTDPEVTNYSLKGCQGKPLPKDLRFIPDPKAGIMIKSVKRAYH
RLCLHCSVDQEGKSVLSEKFILKVRPAFKAVPVVSVSKASYLLREGEEFTVTCTIKDVSS
SVYSTWKRENSQTKLQEKYNSWHHGDFNYERQATLTISSARVNDSGVFMCYANNTFGSAN
VTTTLEVVDKGFINIFPMINTTVFVNDGENVDLIVEYEAFPKPEHQQWIYMNRTFTDKWE
DYPKSENESNIRYVSELHLTRLKGTEGGTYTFLVSNSDVNAAIAFNVYVNIKPEILTYDR
LVNGMLQCVAAGFPEPTIDWYFCPGTEQRCSASVLPVDVQTLNSSGPPFGKLVVQSSIDS
SAFKHNGTVECKAYNDVGKTSAYFNFAFKGNNKEQIHPHTLFTPLLIGFVIVAGMMCIIV
MILTYKYLQKPMYEVQWKVVEEINGNNYVYIDPTQLPYDHKWEFPRNRLSFGKTLGAGAF
GKVVEATAYGLIKSDAAMTVAVKMLKPSAHLTEREALMSELKVLSYLGNHMNIVNLLGAC
TIGGPTLVITEYCCYGDLLNFLRRKRDSFICSKQEDHAEAALYKNLLHSKESSCSDSTNE
YMDMKPGVSYVVPTKADKRRSVRIGSYIERDVTPAIMEDDELALDLEDLLSFSYQVAKGM
AFLASKNCIHRDLAARNILLTHGRITKICDFGLARDIKNDSNYVVKGNARLPVKWMAPES
IFNCVYTFESDVWSYGIFLWELFSLGSSPYPGMPVDSKFYKMIKEGFRMLSPEHAPAEMY
DIMKTCWDADPLKRPTFKQIVQLIEKQISESTNHIYSNLANCSPNRQKPVVDHSVRINSV
GSTASSSQPLLVHDDV

The term “antibody” as used herein refers to a protein comprising at least two heavy (H) chains and two light (L) chains inter-connected by disulfide bonds, which interacts with an antigen. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FR's arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. The term “antibody” includes for example, monoclonal antibodies, human antibodies, humanized antibodies, camelised antibodies and chimeric antibodies. The antibodies can be of any isotype (e.g., IgG, IgE, IgM, IgD, IgA and IgY), class (e.g., Igd, IgG2, IgG3, IgG4, IgA1 and IgA2) or subclass. Both the light and heavy chains are divided into regions of structural and functional homology.

The term “antibody fragment”, as used herein, refers to one or more portions of an antibody that retain the ability to specifically interact with (e.g., by binding, steric hindrance, stabilizing spatial distribution) an antigen. Examples of binding fragments include, but are not limited to, a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al, (1989) Nature 341:544-546), which consists of a VH domain; and an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al, (1988) Science 242:423-426; and Huston et al, (1988) Proc. Natl. Acad. Sci. 85:5879-5883). Such single chain antibodies are also intended to be encompassed within the term “antibody fragment”. These antibody fragments are obtained using conventional techniques known to those of skill in the art, and the fragments are screened for utility in the same manner as are intact antibodies. Antibody fragments can also be incorporated into single domain antibodies, maxibodies, minibodies, intrabodies, diabodies, triabodies, tetrabodies, v-NAR and bis-scFv (see, e.g., Hollinger and Hudson, (2005) Nature Biotechnology 23:1126-1136). Antibody fragments can be grafted into scaffolds based on polypeptides such as Fibronectin type III (Fn3) (see U.S. Pat. No. 6,703,199, which describes fibronectin polypeptide monobodies). Antibody fragments can be incorporated into single chain molecules comprising a pair of tandem Fv segments (VH-CH1-VH-CH1) which, together with complementary light chain polypeptides, form a pair of antigen-binding sites (Zapata et al, (1995) Protein Eng. 8: 1057-1062; and U.S. Pat. No. 5,641,870).

The structures and locations of immunoglobulin variable domains, e.g., CDRs, may be defined using well known numbering schemes, e.g., the Kabat numbering scheme, the Chothia numbering scheme, or a combination of Kabat and Chothia (see, e.g. Sequences of Proteins of Immunological Interest, U.S. Department of Health and Human Services (1991), eds. Kabat et al; Lazikani et at., (1997) J. Mol. Bio. 273:927-948); Kabat et al, (1991) Sequences of Proteins of Immunological Interest, 5th edit., NIH Publication no. 91-3242 U.S. Department of Health and Human Services; Chothia et al., (1987) J. Mol. Biol. 196:901-917; Chothia et al, (1989) Nature 342:877-883; and Al-Lazikani et al, (1997) J. Mol. Biol. 273:927-948; Annals of the New York Academy of Sciences, 764, 47-49 (1995); Nucleic Acids Research, 25, 206-211 (1997).

A “human antibody” or “human antibody fragment”, as used herein, is an antibody and antibody fragment having variable regions in which both the framework and CDR regions are from sequences of human origin. Human antibodies can also be isolated from synthetic libraries or from transgenic mice (e.g. Xenomouse, OmniMouse, Harbour Mouse, ATX-Gx Mouse, Trianni Mouse) provided the respective system yield in antibodies having variable regions in which both the framework and CDR regions are derived from sequences of human origin. Furthermore, if the antibody contains a constant region, the constant region also is derived from such sequences. Human origin includes, e.g., human germline sequences, or mutated versions of human germline sequences or antibody containing consensus framework sequences derived from human framework sequences analysis, for example, as described in Knappik et al, (2000) J Mol Biol 296:57-86).

A “humanized antibody” or “humanized antibody fragment” is defined herein as an antibody molecule, which has constant antibody regions derived from sequences of human origin and the variable antibody regions or parts thereof or only the CDRs are derived from another species. For example, a humanized antibody can be CDR-grafted, wherein the CDRs of the variable domain are from a non-human origin, while one or more frameworks of the variable domain are of human origin and the constant domain (if any) is of human origin.

The term “chimeric antibody” or “chimeric antibody fragment” is defined herein as an antibody molecule, which has constant antibody regions derived from, or corresponding to, sequences found in one species and variable antibody regions derived from another species. Preferably, the constant antibody regions are derived from, or corresponding to, sequences found in humans, and the variable antibody regions (e.g. VH, VL, CDR or FR regions) are derived from sequences found in a non-human animal, e.g. a mouse, rat, rabbit or hamster.

The term “isolated antibody” or “isolated antibody fragment” refers to an antibody or antibody fragment that is substantially free of other antibodies or antibody fragments having different antigenic specificities. Moreover, an isolated antibody or antibody fragment may be substantially free of other cellular material and/or chemicals. Thus, in some aspects, antibodies provided are isolated antibodies, which have been separated from antibodies with a different specificity. An isolated antibody may be a monoclonal antibody. An isolated antibody may be a recombinant monoclonal antibody. An isolated antibody that specifically binds to an epitope, isoform or variant of a target may, however, have cross-reactivity to other related antigens, e.g., from other species (e.g., species homologs).

The term “recombinant antibody” or “recombinant antibody fragment”, as used herein, includes all antibodies or antibody fragment that are prepared, expressed, created or segregated by means not existing in nature. For example, antibodies isolated from a host cell transformed to express the antibody, antibodies selected and isolated from a recombinant, combinatorial human antibody library, and antibodies prepared, expressed, created or isolated by any other means that involve splicing of all or a portion of a human immunoglobulin gene, sequences to other DNA sequences or antibodies isolated from an animal (e.g., a mouse) that is transgenic or transchromosomal for human immunoglobulin genes or a hybridoma prepared therefrom. Preferably, such recombinant antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. A recombinant antibody may be a monoclonal antibody.

The term “monoclonal” as used herein has the meaning typically ascribed to it in the art, namely an antibody or an antibody fragment (or its corresponding functional fragment) arising from a single clone of an antibody-producing cell, recognizing a single epitope on the antigen bound.

As used herein, an antibody “binds specifically to”, “specifically binds to”, is “specific to/for” or “specifically recognizes” an antigen, such as human CD117, if such antibody is able to discriminate between such antigen and one or more reference antigen(s), since binding specificity is not an absolute, but a relative property. For example, a standard ELISA assay or standard flow cytometry assay can be carried out. The scoring may be carried out by standard color development (e.g. secondary antibody with horseradish peroxide and tetramethyl benzidine with hydrogen peroxide) or by binding of a secondary antibody labeled with PE or another dye or marker. The reaction in certain wells is scored by the optical density (OD), for example, at 450 nm or by mean or median fluorescence intensity (MFI) in flow cytometry. Typical background (=negative reaction) may be 0.1 OD; typical positive reaction may be 1 OD. Background and positive reaction MFI are highly dependent on instrument settings. The difference positive/negative can be more than 10-fold. Typically, determination of binding specificity is performed by using not a single reference antigen, but a set of about three to five unrelated antigens, such as milk powder, BSA, transferrin or the like. For flow cytometry various antigen-negative cells can be used. An antibody that specifically binds to an antigen may however have cross-reactivity to the respective orthologous antigen from other species (e.g., species homologs). In certain embodiments such cross-reactivity to an orthologous antigen is even preferred.

As used herein, an antibody has “cross-reactivity” or is “cross-reactive” if it binds to the orthologous antigen from other species. For example, an antibody is cross-reactive if it binds to human CD117 and to cynomolgus CD117.

As used herein, the term “affinity” refers to the strength of interaction between the polypeptide and its target at a single site. Within each site, the binding region of the polypeptide interacts through weak non-covalent forces with its target at numerous sites; the more interactions, the stronger the affinity.

The term “epitope” includes any proteinaceous region which is specifically recognized by an antibody or antibody fragment thereof or otherwise interacts with a molecule. Generally, epitopes are of chemically active surface groupings of molecules such as amino acids or carbohydrate or sugar side chains and generally may have specific three-dimensional structural characteristics, as well as specific charge characteristics. As will be appreciated by one of skill in the art, practically anything to which an antibody can specifically bind could be an epitope.

The term “domain” or “protein domain” refers to a region of a protein's polypeptide chain that forms a functional unit and/or independently forms a three-dimensional structure.

“Compositions” or of the present disclosure may be used for therapeutic or prophylactic applications. The present disclosure, therefore, includes a pharmaceutical composition containing an antibody or antibody fragment as disclosed herein and a pharmaceutically acceptable carrier or excipient therefore. In a related aspect, the present disclosure provides a method for treating inflammatory diseases, autoimmune diseases, hematologic malignancies and potentially other diseases. Such method contains the steps of administering to a subject in need thereof an effective amount of the pharmaceutical composition that contains an antibody or antibody fragment as described herein.

The present disclosure provides therapeutic methods comprising the administration of a therapeutically effective amount of an antibody or antibody fragment as disclosed herein to a subject in need of such treatment. A “therapeutically effective amount” or “effective amount”, as used herein, refers to the amount of an anti-CD117 antibody necessary to elicit the desired biological response. In accordance with the subject disclosure, the therapeutic effective amount is the amount of an anti-CD117 antibody necessary to treat and/or prevent a disease.

“Administered” or “administration” includes but is not limited to delivery of a drug by an injectable form, such as, for example, an intravenous, intramuscular, intradermal or subcutaneous route or mucosal route, for example, as a nasal spray or aerosol for inhalation or as an ingestible solution, capsule or tablet. Preferably, the administration is by an injectable form.

As used herein, “treatment”, “treat” or “treating” and the like refers to clinical intervention in an attempt to alter the natural course of a disease in the subject being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies or antibody fragments according to the preset disclosure are used to delay development of a disease or to slow the progression of a disease.

“Preventing” or “prevention” refers to a reduction in risk of acquiring or developing a disease (i.e., causing at least one of the clinical symptoms of the disease not to develop in a subject that may be exposed to a disease-causing agent, or predisposed to the disease in advance of disease onset). “Prevention” also refers to methods which aim to prevent the onset of a disease or its symptoms or which delay the onset of a disease or its symptoms.

“Subject” or “species” or as used in this context refers to any mammal, including rodents, such as mouse or rat, and primates, such as cynomolgus monkey (Macaca fascicularis), Marmoset monkey (Callithrix jacchus), rhesus monkey (Macaca mulatta) or humans (Homo sapiens). Preferably, the subject is a primate, most preferably a human.

The term “effector function” refers to those biological activities attributable to the Fc region of an antibody, which vary with the antibody isotype. Non-limiting examples of antibody effector functions include C1 q binding and complement dependent cytotoxicity (CDC); Fc receptor binding and antibody-dependent cell-mediated cytotoxicity (ADCC) and/or antibody-dependent cellular phagocytosis (ADCP); down regulation of cell surface receptors (e.g. B cell receptor); and direct cell activation or direct cell inhibition.

“Antibody-dependent cell-mediated cytotoxicity” or “ADCC” refers to a form of cytotoxicity in which antibodies bound onto Fc receptors (FcRs) present on certain cytotoxic cells (e.g. NK cells, neutrophils, and macrophages) enable these cytotoxic effector cells to bind specifically to an antigen-bearing target cell and subsequently kill the target cell with cytotoxins. The primary cells for mediating ADCC, NK cells, express FcγRIII only, whereas monocytes/macrophages express FcγRI, FcγRII, and FcγRIII.

“Complement-dependent cytotoxicity” or “CDC” refers to the lysis of a target cell in the presence of complement. Activation of the classical complement pathway is initiated by the binding of the first component of the complement system (C1q) to antibodies (of the appropriate subclass) of the present disclosure, which are bound to their cognate antigen.

“Antibody-dependent cellular phagocytosis” or “ADCP” refers to a mechanism of elimination of antibody-coated target cells by internalization by phagocytic cells, such as macrophages or dendritic cells.

Throughout this specification, unless the context requires otherwise, the words “comprise”, “have” and “include” and their respective variations such as “comprises”, “comprising”, “has”, “having”, “includes” and “including” will be understood to imply the inclusion of a stated element or integer or group of elements or integers but not the exclusion of any other element or integer or group of elements or integers.

The terms “engineered” or“modified” as used herein includes manipulation of nucleic acids or polypeptides by synthetic means (e.g., by recombinant techniques, in vitro peptide synthesis, by enzymatic or chemical coupling of peptides or some combination of these techniques). Preferably, the antibodies or antibody fragments according to the present disclosure are engineered or modified to improve one or more properties, such as antigen binding, stability, half-life, effector function, immunogenicity, safety and the like.

“Variant” as used herein refers to a polypeptide that differs from a reference polypeptide by one or more modifications for example amino acid substitutions, insertions or deletions. Variant polypeptides typically retain most of the properties of the reference polypeptide, e.g. binding to the target antigen, but introduce a novel, additional feature or property, e.g. the variant polypeptide has a higher affinity to the target antigen compared to the reference polypeptide or the variant polypeptide is a humanized version of the reference polypeptide.

The term “amino acid mutation” as used herein is meant to encompass amino acid substitutions, deletions, insertions, and modifications. Any combination of substitution, deletion, insertion, and modification can be made as long as the final construct possesses the desired characteristics, e.g., reduced binding to an Fc receptor. Amino acid sequence deletions and insertions include N-and/or C-terminal deletions and insertions of amino acid residues. Particular amino acid mutations are amino acid substitutions. Amino acid substitutions include replacement by non-naturally occurring amino acids or by naturally occurring amino acid derivatives of the twenty standard amino acids. Amino acid mutations can be generated using genetic or chemical methods well known in the art. Genetic methods may include site-directed mutagenesis, PCR, gene synthesis and the like. It is contemplated that methods of altering the side chain group of an amino acid residue by methods other than genetic engineering, such as chemical modification, may also be useful. Various designations may be used herein to indicate the same amino acid mutation. For example, a substitution of glycine at position 237 of the antibody Fc region to alanine can be indicated as 237A, G237, G237A, or Gly237Ala.

The term “EC50” as used herein, refers to the concentration of an antibody or antibody fragment, which induces a response in an assay half-way between the baseline and maximum. It therefore represents the antibody or ligand concentration at which 50% of the maximal effect is observed.

The term “Ka”, as used herein refers to the association rate of a particular antibody-antigen interaction.

The term “Kd” as used herein, refers to the dissociation rate of a particular antibody-antigen interaction. Kd values for antibodies can be determined using methods well established in the art.

The term “KD” as used herein, refer to the dissociation constant of a particular antibody-antigen interaction, which is obtained from the ratio of Kd to Ka (i.e., Kd/Ka) and is expressed as a molar concentration. A preferred method for determining the Kd of an antibody is by using surface plasmon resonance, preferably using a biosensor system, such as a Biacore system, or by using biolayer interferometry with the Octet BLI instrument.

The terms “inhibition” or “inhibit” or “reduction” or “reduce” or “neutralization” or “neutralize” refer to a decrease or cessation of any phenotypic characteristic (such as binding or a biological activity or function) or to the decrease or cessation in the incidence, degree, or likelihood of that characteristic. “Inhibition”, “reduction” or “neutralization” needs not to be complete as long as it is detectable using an appropriate assay. In some embodiments, by “reduce” or “inhibit” or “neutralize” is meant the ability to cause a decrease of 20% or greater. In another embodiment, by “reduce” or “inhibit” or “neutralize” is meant the ability to cause a decrease of 50% or greater. In yet another embodiment, by “reduce” or “inhibit” or “neutralize” is meant the ability to cause an overall decrease of 75%, 85%, 90%, 95%, or greater.

The term “antagonistic” antibody as used herein refers to an antibody or antibody fragment that interacts with an antigen and partially or fully inhibits or neutralizes a biological activity or function or any other phenotypic characteristic of a target antigen.

A “wild-type” protein is a version or variant of the protein as it is found in nature. An amino acid sequence of a wildtype protein, e.g., a Fc region of a human IgG1 antibody, is the amino acid sequence of the protein as it occurs in nature. Due to allotypic differences, there can be more than one amino acid sequence for a wildtype protein. For example, there are several allotypes of naturally occurring human IGg1 heavy chain constant regions (see, e.g., Jeffries et at. (2009) mAbs 1:1).

The “Fc region” is used to define the C-terminal region of an immunoglobulin heavy chain. The Fc region of an immunoglobulin generally comprises two constant domains, a CH2 domain and a CH3 domain. Although the boundaries of the Fc region of an IgG heavy chain might vary slightly, the human IgG heavy chain Fc region is usually defined to extend from Cys226, or from Pro230, to the C-terminus of the heavy chain. However, the C-terminal lysine (Lys447) of the Fc region may or may not be present. Unless otherwise specified herein, numbering of amino acid residues in the Fc region is according to the EU numbering system, also called the EU index, as described in Kabat et al, Sequences of Proteins of Immunological Interest, 5th Ed. Public Health Service, National Institutes of Health, Bethesda, MD, 1991. Various Fc modifications are commonly used. For a review see for example Antibodies (2020) 9: 64. Silencing functions include (numbering according EU index) the LALA (L234A/L235A), the PA-LALA (L234A/L235A/P329A) and the PG-LALA (L234A/L235A/P329G) mutations, as well as the AEASS mutations (L234A/L235E/G237A/A330S/P331S). A preferred FC modification is PA-LALA.

### EMBODIMENTS OF THE INVENTION

### Polypeptides

In certain embodiments, the present disclosure relates to antibodies or antibody fragments that are specific for CD117. In certain embodiments, the present disclosure relates to antibodies or antibody fragments that are specific for human CD117. In certain embodiments, the present disclosure relates to humanized antibodies or antibody fragments that are specific for CD117. In certain embodiments, the present disclosure relates to humanized antibodies or antibody fragments that are specific for human CD117.

In certain embodiments, the present disclosure relates to humanized antibodies or antibody fragments that are specific fora polypeptide consisting of the amino acid sequence of SEQ ID No. 1. In certain embodiments, the present disclosure relates to humanized antibodies or antibody fragments that are specific for a polypeptide comprising the amino acid sequence of SEQ ID No. 1.

In certain embodiments, the present disclosure relates to a humanized antibody or antibody fragment specific for human CD117, wherein said antibody or antibody fragment comprises a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9, wherein said humanized antibody or antibody fragment has at least a mutation in which the asparagine in the LCDR1 region (SEQ ID NO: 7) is replaced by glutamic acid or tyrosine.

In certain embodiments, the present disclosure relates to a humanized antibody or antibody fragment specific for human CD117, wherein said antibody or antibody fragment comprises a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9, wherein said humanized antibody or antibody fragment has at least a mutation in which the asparagine in the LCDR1 region (SEQ ID NO: 7) is replaced by glutamic acid or tyrosine, and wherein said humanized antibody or antibody fragment has one or more of the additional following mutations:
a) the first asparagine in the HCDR2 region (SEQ ID NO: 5) is replaced by a serine,
b) the second asparagine in the LCDR3 region (SEQ ID NO: 9) is replaced by lysine, and/or
c) the aspartic acid in the LCDR3 region (SEQ ID NO: 9) is replaced by glutamic acid.

In certain embodiments, the present disclosure relates to a humanized antibody or antibody fragment as described above, wherein said humanized antibody or antibody fragment comprises:
a) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 95,
b) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 81, the LCDR2 region of SEQ ID NO: 8 and the LCDR3 region of SEQ ID NO: 113,
c) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 113,
d) a variable heavy chain comprising a HCDR1 region of SEQ ID NO: 4, a HCDR2 region of SEQ ID NO: 71, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 95,
e) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 81, the LCDR2 region of SEQ ID NO: 8 and the LCDR3 region of SEQ ID NO: 95,
f) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO:9, or
g) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 81, the LCDR2 region of SEQ ID NO: 8 and the LCDR3 region of SEQ ID NO: 9.

In certain embodiments, the present disclosure relates to a humanized antibody or antibody fragment as described above, wherein said humanized antibody or antibody fragment comprises
a) a variable heavy chain of SEQ ID NO: 22 and a variable light chain of SEQ ID NO: 98,
b) a variable heavy chain of SEQ ID NO: 22 and a variable light chain of SEQ ID NO: 99,
c) a variable heavy chain of SEQ ID NO: 22 and a variable light chain of SEQ ID NO: 100,
d) a variable heavy chain of SEQ ID NO: 70 and a variable light chain of SEQ ID NO: 98,
e) a variable heavy chain of SEQ ID NO: 22 and a variable light chain of SEQ ID NO: 97,
f) a variable heavy chain of SEQ ID NO: 22 and a variable light chain of SEQ ID NO: 61, or
g) a variable heavy chain of SEQ ID NO: 22 and a variable light chain of SEQ ID NO: 62.

In certain embodiments, the present disclosure relates to a humanized antibody or antibody fragment specific for human CD117, wherein said antibody or antibody fragment comprises a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9, wherein said humanized antibody or antibody fragment has at least the two following mutations:
a) the asparagine in the LCDR1 region (SEQ ID NO: 7) is replaced by glutamic acid or tyrosine, and
b) the aspartic acid in the LCDR3 region (SEQ ID NO: 9) is replaced by glutamic acid.

In certain embodiments, said humanized antibody or antibody fragment specific for human CD117 has one or more of the additional following mutations:
a) the second asparagine in the LCDR3 region (SEQ ID NO: 9) is replaced by lysine, and/or
b) the first asparagine in the HCDR2 region (SEQ ID NO: 5) is replaced by serine.

In certain embodiments, the present disclosure relates to a humanized antibody or antibody fragment specific for human CD117, wherein said antibody or antibody fragment comprises a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9, wherein said humanized antibody or antibody fragment has the two following mutations:
a) the asparagine in the LCDR1 region (SEQ ID NO: 7) is replaced by glutamic acid, and
b) the aspartic acid in the LCDR3 region (SEQ ID NO: 9) is replaced by glutamic acid.

In certain embodiments, the present disclosure relates to a humanized antibody or antibody fragment specific for human CD117, wherein said antibody or antibody fragment comprises a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9, wherein said humanized antibody or antibody fragment has the three following mutations:
a) the asparagine in the LCDR1 region (SEQ ID NO: 7) is replaced by tyrosine,
b) the second asparagine in the LCDR3 region (SEQ ID NO: 9) is replaced by lysine, and
c) the aspartic acid in the LCDR3 region (SEQ ID NO: 9) is replaced by glutamic acid.

In certain embodiments, the present disclosure relates to a humanized antibody or antibody fragment specific for human CD117, wherein said antibody or antibody fragment comprises a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9, wherein said humanized antibody or antibody fragment has the two following mutations:
a) the asparagine in the LCDR1 region (SEQ ID NO: 7) is replaced by glutamic acid,
b) the second asparagine in the LCDR3 region (SEQ ID NO: 9) is replaced by lysine, and
c) the aspartic acid in the LCDR3 region (SEQ ID NO: 9) is replaced by glutamic acid.

In certain embodiments, the present disclosure relates to a humanized antibody or antibody fragment specific for human CD117, wherein said antibody or antibody fragment comprises a variable heavy chain comprising a HCDR1 region of SEQ ID NO: 4, a HCDR2 region of SEQ ID NO: 5, a HCDR3 region of SEQ ID NO: 6 and a variable heavy chain comprising, the LCDR1 region of SEQ ID NO: 7, the LCDR2 region of SEQ ID NO: 8 and the LCDR3 region of SEQ ID NO: 9, wherein said humanized antibody or antibody fragment has the two following mutations:
a) the first asparagine in the HCDR2 region (SEQ ID NO:5) is replaced by serine,
b) the asparagine in the LCDR1 region (SEQ ID NO: 7) is replaced by glutamic acid, and
c) the aspartic acid in the LCDR3 region (SEQ ID NO: 9) is replaced by glutamic acid.

In certain embodiments, the present disclosure relates to a humanized antibody or antibody fragment specific for human CD117, wherein said antibody or antibody fragment comprises a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9, wherein the humanized antibody or antibody fragment comprises a mutation by which the asparagine in the LCDR1 region (SEQ ID NO: 7) is replaced by glutamic acid, histidine, glutamine or serine.

In certain embodiments, the present disclosure relates to a humanized antibody or antibody fragment specific for human CD117, wherein said antibody or antibody fragment comprises a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9, wherein the humanized antibody or antibody fragment comprises a mutation by which the second asparagine in the LCDR3 region (SEQ ID NO: 9) is replaced by lysine or arginine.

In certain embodiments, the present disclosure relates to a humanized antibody or antibody fragment specific for human CD117, wherein said antibody or antibody fragment comprises a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9, wherein the humanized antibody or antibody fragment comprises a mutation by which the aspartic acid in the LCDR3 region (SEQ ID NO: 9) is replaced by glutamic acid.

In certain embodiments, the present disclosure relates to a humanized antibody or antibody fragment specific for human CD117, wherein said antibody or antibody fragment comprises a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9, wherein the humanized antibody or antibody fragment comprises the two following mutations:
the asparagine in the LCDR1 region (SEQ ID NO: 7) is replaced by glutamic acid, histidine, glutamine or serine, and
the second asparagine in the LCDR3 region (SEQ ID NO: 9) is replaced by lysine or arginine.

In certain embodiments, the present disclosure relates to a humanized antibody or antibody fragment specific for human CD117, wherein said antibody or antibody fragment comprises a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9, wherein the humanized antibody or antibody fragment comprises the two following mutations:
the asparagine in the LCDR1 region (SEQ ID NO: 7) is replaced by glutamic acid, histidine, glutamine or serine, and
the aspartic acid in the LCDR3 region (SEQ ID NO: 9) is replaced by glutamic acid.

In certain embodiments, the present disclosure relates to a humanized antibody or antibody fragment specific for human CD117, wherein said antibody or antibody fragment comprises a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9, wherein the humanized antibody or antibody fragment comprises the two following mutations:
the second asparagine in the LCDR3 region (SEQ ID NO: 9) is replaced by lysine or arginine, and
the aspartic acid in the LCDR3 region (SEQ ID NO: 9) is replaced by glutamic acid.

In certain embodiments, the present disclosure relates to a humanized antibody or antibody fragment specific for human CD117, wherein said antibody or antibody fragment comprises a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9, wherein the humanized antibody or antibody fragment comprises the three following mutations:
the asparagine in the LCDR1 region (SEQ ID NO: 7) is replaced by glutamic acid, histidine, glutamine or serine,
the second asparagine in the LCDR3 region (SEQ ID NO:9) is replaced by lysine or arginine, and
the aspartic acid in the LCDR3 region (SEQ ID NO: 9) is replaced by glutamic acid.

In certain embodiments, the present disclosure relates to a humanized antibody or antibody fragment specific for human CD117, wherein said antibody or antibody fragment comprises a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9, wherein the humanized antibody or antibody fragment comprises a mutation by which the first leucine in the LCDR2 region (SEQ ID NO: 8) is replaced by lysine, histidine, glutamic acid, serine or glutamine.

In certain embodiments, the present disclosure relates to a humanized antibody or antibody fragment specific for human CD117, wherein said antibody or antibody fragment comprises a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9, wherein the humanized antibody or antibody fragment comprises the four following mutations:
the asparagine in the LCDR1 region (SEQ ID NO: 7) is replaced by glutamic acid, histidine, glutamine or serine,
the second asparagine in the LCDR3 region (SEQ ID NO: 9) is replaced by lysine or arginine,
the aspartic acid in the LCDR3 region (SEQ ID NO: 9) is replaced by glutamic acid, and
the first leucine in the LCDR2 region (SEQ ID NO: 8) is replaced by lysine, histidine, glutamic acid, serine or glutamine.

In certain embodiments, the present disclosure relates to a humanized antibody or antibody fragment specific for human CD117, wherein said antibody or antibody fragment comprises a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9, wherein the humanized antibody or antibody fragment comprises a mutation by which the first asparagine in the HCDR2 region (SEQ ID NO: 5) is replaced by serine or glutamine.

In certain embodiments, the present disclosure relates to a humanized antibody or antibody fragment specific for human CD117, wherein said antibody or antibody fragment comprises a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9, wherein the humanized antibody or antibody fragment comprises the four following mutations:
the asparagine in the LCDR1 region (SEQ ID NO: 7) is replaced by glutamic acid, histidine, glutamine or serine,
the second asparagine in the LCDR3 region (SEQ ID NO: 9) is replaced by lysine or arginine,
the aspartic acid in the LCDR3 region (SEQ ID NO: 9) is replaced by glutamic acid, and
the first asparagine in the HCDR2 region (SEQ ID NO: 5) is replaced by serine or glutamine.

In certain embodiments, the present disclosure relates to a humanized antibody or antibody fragment specific for human CD117, wherein said antibody or antibody fragment comprises a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9, wherein the humanized antibody or antibody fragment comprises the five following mutations:
the asparagine in the LCDR1 region (SEQ ID NO: 7) is replaced by glutamic acid, histidine, glutamine or serine,
the second asparagine in the LCDR3 region (SEQ ID NO: 9) is replaced by lysine or arginine,
the aspartic acid in the LCDR3 region (SEQ ID NO: 9) is replaced by glutamic acid,
the first leucine in the LCDR2 region (SEQ ID NO: 8) is replaced by lysine, histidine, glutamic acid, serine or glutamine, and
the first asparagine in the HCDR2 region (SEQ ID NO: 5) is replaced by serine or glutamine.

In certain embodiments, the present disclosure relates to a humanized antibody or antibody fragment, wherein said humanized antibody or antibody fragment comprises:
a) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 95,
b) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 81, the LCDR2 region of SEQ ID NO: 8 and the LCDR3 region of SEQ ID NO: 113,
c) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 113,
d) a variable heavy chain comprising a HCDR1 region of SEQ ID NO: 4, a HCDR2 region of SEQ ID NO: 71, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 95,
e) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 115,
f) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO:115,
g) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 121 and the LCDR3 region of SEQ ID NO: 115,
h) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 123 and the LCDR3 region of SEQ ID NO: 115,
i) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 125 and the LCDR3 region of SEQ ID NO: 115,
j) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 127 and the LCDR3 region of SEQ ID NO: 115, or,
k) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 113.

In certain embodiments, the present disclosure relates to a humanized antibody or antibody fragment, wherein said humanized antibody or antibody fragment comprises:
a) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 95,
b) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 81, the LCDR2 region of SEQ ID NO: 8 and the LCDR3 region of SEQ ID NO: 113,
c) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 113, or
d) a variable heavy chain comprising a HCDR1 region of SEQ ID NO: 4, a HCDR2 region of SEQ ID NO: 71, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 95.

In certain embodiments, the present disclosure relates to a humanized antibody or antibody fragment, wherein said humanized antibody or antibody fragment comprises a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 95.

In certain embodiments, the present disclosure relates to a humanized antibody or antibody fragment, wherein said humanized antibody or antibody fragment comprises a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 81, the LCDR2 region of SEQ ID NO: 8 and the LCDR3 region of SEQ ID NO: 113.

In certain embodiments, the present disclosure relates to a humanized antibody or antibody fragment, wherein said humanized antibody or antibody fragment comprises a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 113.

In certain embodiments, the present disclosure relates to a humanized antibody or antibody fragment, wherein said humanized antibody or antibody fragment comprises a variable heavy chain comprising a HCDR1 region of SEQ ID NO: 4, a HCDR2 region of SEQ ID NO: 71, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 95.

In certain embodiments, the present disclosure relates to a humanized antibody or antibody fragment, wherein said humanized antibody or antibody fragment comprises a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 45, the LCDR2 region of SEQ ID NO: 8 and the LCDR3 region of SEQ ID NO: 113.

In certain embodiments, the present disclosure relates to a humanized antibody or antibody fragment, wherein said humanized antibody or antibody fragment comprises a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 44, the LCDR2 region of SEQ ID NO: 8 and the LCDR3 region of SEQ ID NO: 113.

In certain embodiments, the present disclosure relates to a humanized antibody or antibody fragment, wherein said humanized antibody or antibody fragment comprises a variable heavy chain comprising a HCDR1 region of SEQ ID NO: 4, a HCDR2 region of SEQ ID NO:5, a HCDR3 region of SEQ ID NO: 6 and a variable light chain comprising, the LCDR1 region of SEQ ID NO: 109, the LCDR2 region of SEQ ID NO: 8 and the LCDR3 region of SEQ ID NO: 113.

In certain embodiments, the present disclosure relates to a humanized antibody or antibody fragment, wherein said humanized antibody or antibody fragment comprises a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 115.

In certain embodiments, the present disclosure relates to a humanized antibody or antibody fragment, wherein said humanized antibody or antibody fragment comprises a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 115.

In certain embodiments, the present disclosure relates to a humanized antibody or antibody fragment, wherein said humanized antibody or antibody fragment comprises a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 121 and the LCDR3 region of SEQ ID NO: 115.

In certain embodiments, the present disclosure relates to a humanized antibody or antibody fragment, wherein said humanized antibody or antibody fragment comprises a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 123 and the LCDR3 region of SEQ ID NO: 115.

In certain embodiments, the present disclosure relates to a humanized antibody or antibody fragment, wherein said humanized antibody or antibody fragment comprises a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 125 and the LCDR3 region of SEQ ID NO: 115.

In certain embodiments, the present disclosure relates to a humanized antibody or antibody fragment, wherein said humanized antibody or antibody fragment comprises a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 127 and the LCDR3 region of SEQ ID NO: 115.

In certain embodiments, the present disclosure relates to a humanized antibody or antibody fragment, wherein said humanized antibody or antibody fragment comprises a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO:8 and the LCDR3 region of SEQ ID NO: 113.

In certain embodiments, the present disclosure relates to a humanized antibody or antibody fragment, wherein said humanized antibody or antibody fragment comprises:
a) a variable heavy chain of SEQ ID NO: 22 and a variable light chain of SEQ ID NO: 98,
b) a variable heavy chain of SEQ ID NO: 22 and a variable light chain of SEQ ID NO: 99,
c) a variable heavy chain of SEQ ID NO: 22 and a variable light chain of SEQ ID NO: 100,
d) a variable heavy chain of SEQ ID NO: 70 and a variable light chain of SEQ ID NO: 98,
e) a variable heavy chain of SEQ ID NO: 22 and a variable light chain of SEQ ID NO: 105,
f) a variable heavy chain of SEQ ID NO: 106 and a variable light chain of SEQ ID NO: 105,
g) a variable heavy chain of SEQ ID NO: 106 and a variable light chain of SEQ ID NO: 117,
h) a variable heavy chain of SEQ ID NO: 106 and a variable light chain of SEQ ID NO: 118,
i) a variable heavy chain of SEQ ID NO: 106 and a variable light chain of SEQ ID NO: 119,
j) a variable heavy chain of SEQ ID NO: 106 and a variable light chain of SEQ ID NO: 120, or
k) a variable heavy chain of SEQ ID NO: 106 and a variable light chain of SEQ ID NO: 100.

In certain embodiments, the present disclosure relates to a humanized antibody or antibody fragment, wherein said humanized antibody or antibody fragment comprises:
a) a variable heavy chain of SEQ ID NO: 22 and a variable light chain of SEQ ID NO: 98,
b) a variable heavy chain of SEQ ID NO: 22 and a variable light chain of SEQ ID NO: 99,
c) a variable heavy chain of SEQ ID NO: 22 and a variable light chain of SEQ ID NO: 100, or
d) a variable heavy chain of SEQ ID NO: 70 and a variable light chain of SEQ ID NO: 98.

In certain embodiments, the present disclosure relates to a humanized antibody or antibody fragment, wherein said humanized antibody or antibody fragment comprises a variable heavy chain of SEQ ID NO: 22 and a variable light chain of SEQ ID NO: 98.

In certain embodiments, the present disclosure relates to a humanized antibody or antibody fragment, wherein said humanized antibody or antibody fragment comprises a variable heavy chain of SEQ ID NO: 22 and a variable light chain of SEQ ID NO: 99.

In certain embodiments, the present disclosure relates to a humanized antibody or antibody fragment, wherein said humanized antibody or antibody fragment comprises a variable heavy chain of SEQ ID NO: 22 and a variable light chain of SEQ ID NO: 100.

In certain embodiments, the present disclosure relates to a humanized antibody or antibody fragment, wherein said humanized antibody or antibody fragment comprises a variable heavy chain of SEQ ID NO: 70 and a variable light chain of SEQ ID NO: 98.

In certain embodiments, the present disclosure relates to a humanized antibody or antibody fragment, wherein said humanized antibody or antibody fragment comprises a variable heavy chain of SEQ ID NO: 22 and a variable light chain of SEQ ID NO: 101.

In certain embodiments, the present disclosure relates to a humanized antibody or antibody fragment, wherein said humanized antibody or antibody fragment comprises a variable heavy chain of SEQ ID NO: 22 and a variable light chain of SEQ ID NO: 102.

In certain embodiments, the present disclosure relates to a humanized antibody or antibody fragment, wherein said humanized antibody or antibody fragment comprises a variable heavy chain of SEQ ID NO: 22 and a variable light chain of SEQ ID NO: 103.

In certain embodiments, the present disclosure relates to a humanized antibody or antibody fragment, wherein said humanized antibody or antibody fragment comprises a variable heavy chain of SEQ ID NO: 22 and a variable light chain of SEQ ID NO: 105.

In certain embodiments, the present disclosure relates to a humanized antibody or antibody fragment, wherein said humanized antibody or antibody fragment comprises a variable heavy chain of SEQ ID NO: 106 and a variable light chain of SEQ ID NO: 105.

In certain embodiments, the present disclosure relates to a humanized antibody or antibody fragment, wherein said humanized antibody or antibody fragment comprises a variable heavy chain of SEQ ID NO: 106 and a variable light chain of SEQ ID NO: 117.

In certain embodiments, the present disclosure relates to a humanized antibody or antibody fragment, wherein said humanized antibody or antibody fragment comprises a variable heavy chain of SEQ ID NO: 106 and a variable light chain of SEQ ID NO: 118.

In certain embodiments, the present disclosure relates to a humanized antibody or antibody fragment, wherein said humanized antibody or antibody fragment comprise a variable heavy chain of SEQ ID NO: 106 and a variable light chain of SEQ ID NO: 119.

In certain embodiments, the present disclosure relates to a humanized antibody or antibody fragment, wherein said humanized antibody or antibody fragment comprises a variable heavy chain of SEQ ID NO: 106 and a variable light chain of SEQ ID NO: 120.

In certain embodiments, the present disclosure relates to a humanized antibody or antibody fragment, wherein said humanized antibody or antibody fragment comprises a variable heavy chain of SEQ ID NO: 106 and a variable light chain of SEQ ID NO: 100.

In certain embodiments, the present disclosure relates to a humanized antibody or antibody fragment, wherein said humanized antibody or antibody fragment comprises 6 six CDRs of one of the antibodies disclosed in Table 18.

In certain embodiments, the present disclosure relates to a humanized antibody or antibody fragment, wherein said humanized antibody or antibody fragment comprises 6 six CDRs as defined by Kabat of one of the antibodies disclosed in Table 18.

In certain embodiments, the present disclosure relates to a humanized antibody or antibody fragment, wherein said humanized antibody or antibody fragment comprises 6 six CDRs as defined by IMGT of one of the antibodies disclosed in Table 18.

In certain embodiments, the present disclosure relates to a humanized antibody or antibody fragment, wherein said humanized antibody or antibody fragment comprises a variable heavy chain and a variable light chain of one of the antibodies disclosed in Table 18.

In certain embodiments, the present disclosure relates to a humanized antibody or antibody fragment, wherein said humanized antibody or antibody fragment is a monoclonal antibody or antibody fragment.

In certain embodiments, the present disclosure relates to a humanized antibody or antibody fragment, wherein said humanized antibody or antibody fragment is a recombinant antibody or antibody fragment.

In certain embodiments, the present disclosure relates to a humanized antibody or antibody fragment, wherein said humanized antibody or antibody fragment is of the IgG isotype.

In certain embodiments, the present disclosure relates to a humanized antibody or antibody fragment, wherein said humanized antibody or antibody fragment is of the IgG1 class.

In certain embodiments, the present disclosure relates to a humanized antibody or antibody fragment, wherein said humanized antibody or antibody fragment is of the human IgG1 class.

The isolated antibody or antibody fragment according to the present disclosure may or may not be fused to one or more other amino acid residues, polypeptides or moieties. Such a fusion protein may be prepared in any suitable manner, including genetically or chemically approaches.

Said linked moieties may contain secretory or leader sequences, sequences that aid detection, expression, separation or purification, or sequences that confer to increased protein stability, for example, during recombinant production. Non-limiting examples of potential moieties include beta-galactosidase, glutathione-S-transferase, luciferase, a T7 polymerase fragment, a secretion signal peptide, an antibody or antibody fragment, a toxin, a cytokine, a chemokine, a reporter enzyme, a moiety being capable of binding a metal ion like a poly-histidine tag, a tag suitable for detection and/or purification, a homo- or hetero-association domain, a moiety which increases solubility of a protein, or a moiety which comprises an enzymatic cleavage site.

Accordingly, the isolated antibody or antibody fragment according to the present disclosure may optionally contain one or more moieties for binding to other targets or target proteins of interest. It should be clear that such further moieties may or may not provide further functionality to the antibody and may or may not modify the properties of the isolated antibody or antibody fragment according to the present disclosure.

### Nucleic Acids

In an embodiment, the present disclosure relates to a nucleic acid composition comprising a nucleic acid sequence or a plurality of nucleic acid sequences encoding an isolated antibody or antibody fragment specific for human CD117, wherein said antibody or antibody fragment comprises:
a) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 95,
b) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 81, the LCDR2 region of SEQ ID NO: 8 and the LCDR3 region of SEQ ID NO: 113,
c) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 113,
d) a variable heavy chain comprising a HCDR1 region of SEQ ID NO: 4, a HCDR2 region of SEQ ID NO: 71, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 95,
e) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 115,
f) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 115,
g) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 121 and the LCDR3 region of SEQ ID NO: 115,
h) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 123 and the LCDR3 region of SEQ ID NO: 115,
i) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 125 and the LCDR3 region of SEQ ID NO: 115,
j) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 127 and the LCDR3 region of SEQ ID NO: 115,
k) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 113,
l) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 81, the LCDR2 region of SEQ ID NO: 8 and the LCDR3 region of SEQ ID NO: 95,
m) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 9, or
n) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 81, the LCDR2 region of SEQ ID NO: 8 and the LCDR3 region of SEQ ID NO: 9.

In an embodiment, the present disclosure relates to a nucleic acid composition comprising a nucleic acid sequence or a plurality of nucleic acid sequences encoding an isolated antibody or antibody fragment specific for human CD117, wherein said antibody or antibody fragment comprises
a) a variable heavy chain of SEQ ID NO: 22 and a variable light chain of SEQ ID NO: 98,
b) a variable heavy chain of SEQ ID NO: 22 and a variable light chain of SEQ ID NO: 99,
c) a variable heavy chain of SEQ ID NO: 22 and a variable light chain of SEQ ID NO: 100,
d) a variable heavy chain of SEQ ID NO: 70 and a variable light chain of SEQ ID NO: 98,
e) a variable heavy chain of SEQ ID NO: 22 and a variable light chain of SEQ ID NO: 105,
f) a variable heavy chain of SEQ ID NO: 106 and a variable light chain of SEQ ID NO: 105,
g) a variable heavy chain of SEQ ID NO: 106 and a variable light chain of SEQ ID NO: 117,
h) a variable heavy chain of SEQ ID NO: 106 and a variable light chain of SEQ ID NO: 118,
i) a variable heavy chain of SEQ ID NO: 106 and a variable light chain of SEQ ID NO: 119,
j) a variable heavy chain of SEQ ID NO: 106 and a variable light chain of SEQ ID NO: 120,
k) a variable heavy chain of SEQ ID NO: 106 and a variable light chain of SEQ ID NO: 100,
l) a variable heavy chain of SEQ ID NO: 22 and a variable light chain of SEQ ID NO: 97,
m) a variable heavy chain of SEQ ID NO: 22 and a variable light chain of SEQ ID NO: 61, or
n) a variable heavy chain of SEQ ID NO: 22 and a variable light chain of SEQ ID NO: 62.

In an embodiment, the present disclosure relates to a nucleic acid composition comprising a nucleic acid sequence or a plurality of nucleic acid sequences encoding an isolated antibody or antibody fragment specific for human CD117, wherein said antibody or antibody fragment comprises 6 six CDRs as defined by Kabat of one of the antibodies disclosed in Table 18.

In an embodiment, the present disclosure relates to a nucleic acid composition comprising a nucleic acid sequence or a plurality of nucleic acid sequences encoding an isolated antibody or antibody fragment specific for human CD117, wherein said antibody or antibody fragment comprises 6 six CDRs as defined by IMGT of one of the antibodies disclosed in Table 18.

In an embodiment, the present disclosure relates to a nucleic acid composition comprising a nucleic acid sequence or a plurality of nucleic acid sequences encoding an isolated antibody or antibody fragment specific for human CD117, wherein said antibody or antibody fragment comprises a variable heavy chain and a variable light chain of one of the antibodies disclosed in Table 18.

In an embodiment, said nucleic acid composition and/or said nucleic acid sequence and/or said plurality of nucleic acid sequences are isolated.

### Vectors

In an embodiment, the present disclosure provides a vector composition comprising a vector or a plurality of vectors comprising a nucleic acid composition comprising a nucleic acid sequence or a plurality of nucleic acid sequences encoding humanized antibody or antibody fragment specific for human CD117 according to the present disclosure.

In an embodiment, the present disclosure provides a vector composition comprising a vector or a plurality of vectors comprising a nucleic acid composition comprising a nucleic acid sequence or a plurality of nucleic acid sequences encoding any one of the humanized antibody or antibody fragment specific for human CD117 disclosed in Table 18.

### Host Cells

In an embodiment, the present disclosure provides a host cell comprising a vector composition comprising a vector or a plurality of vectors comprising a nucleic acid composition comprising a nucleic acid sequence or a plurality of nucleic acid sequences encoding a humanized antibody or antibody fragment specific for human CD117 according to the present disclosure.

In an embodiment, the present disclosure refers to a host cell comprising a vector composition comprising a vector or a plurality of vectors comprising a nucleic acid composition comprising a nucleic acid sequence or a plurality of nucleic acid sequences encoding any one of the humanized antibody or antibody fragment specific for human CD117 disclosed in Table 18.

In an embodiment, the host cell according to the present disclosure is able to express the humanized antibody or antibody fragment specific for human CD117 encoded by the vector composition or the nucleic acid composition.

In a further embodiment, the host cell is an isolated host cell. In a further embodiment, said host cell is a mammalian cell. In an embodiment, said mammalian cell is a human cell. In another embodiment, said mammalian cell is a CHO cell. In an embodiment, said cell is a HEK cell. In another embodiment, said cell is a PERC.6 cell. In an embodiment, said cell is a HKB11 cell.

The skilled artisan will realize that the nucleic acid sequence or the plurality of nucleic acid sequences encoding the heavy and/or light chain of an antibody or antibody fragment of the present disclosure can be cloned into different vectors or into the same vector.

The vectors can be introduced into the appropriate host cells such as prokaryotic (e.g., bacterial) or eukaryotic (e.g., yeast or mammalian) cells by methods well known in the art (see e.g., “Current Protocol in Molecular Biology”, Ausubel et al. (eds.), Greene Publishing Assoc and John Wiley Interscience, New York, 1989 and 1992). Numerous cloning vectors are known to those of skill in the art, and the selection of an appropriate cloning vector is a matter of choice. The gene can be placed under the control of a promoter, ribosome binding site (for bacterial expression) and, optionally, an operator (collectively referred to herein as “control” elements), so that the nucleic acid sequence encoding the desired protein is transcribed into RNA in the host cell transformed by a vector containing this expression construction. The coding sequence may or may not contain a signal peptide or leader sequence. Upon expression in host cells, the antibodies or antibody fragments of the present disclosure are obtained. These steps can be achieved in different ways, as will be known by the person skilled in the art. In general, such steps typically include transforming or transfecting a suitable host cell with a nucleic acid composition or vector composition or an infectious particle, which encodes the antibody, or antibody fragments. Further, such steps typically include culturing said host cells under conditions suitable for the proliferation (multiplication, growth) of said host cells and a culturing step under conditions suitable for the production (expression, synthesis) of the encoded antibody or antibody fragment. The culturing of host cells under conditions suitable for proliferation or expression is typically accomplished in the presence of media comprising components suitable for cell growth or induction of expression. In particular, embodiments, the methods for the production of the antibodies or antibody fragments of the present disclosure further comprise the step of isolating and purifying the produced antibody or antibody fragment from the host cells or medium. If the expression system secretes the protein into growth media, the protein can be purified directly from the media. If the protein is not secreted, it is isolated from cell lysates or recovered from the cell membrane fraction. The selection of the appropriate growth conditions and recovery methods are within the skill of the art. The antibody or antibody fragment of the present disclosure can then be purified by a number of techniques as known to the person skilled in the art.

In an embodiment, the present disclosure refers to a method of producing a humanized antibody or antibody fragment specific for human CD117 of any of the antibodies disclosed in Table 18. In an embodiment, a method of producing an isolated antibody or antibody fragment according to the present disclosure is provided, wherein the method comprises culturing a host cell comprising a vector composition comprising a vector or a plurality of vectors comprising a nucleic acid composition comprising a nucleic acid sequence or a plurality of nucleic acid sequences encoding an antibody or antibody fragment according to the present disclosure, under conditions suitable for expression of the antibody or antibody fragment, and isolating the antibody or antibody fragment from the host cell or host cell culture medium. An antibody or antibody fragment isolated as described herein may be purified techniques know in the art, such as high-performance liquid chromatography (HPLC), ion exchange chromatography, gel electrophoresis, affinity chromatography, size exclusion chromatography, and the like. The conditions used to purify a particular antibody or antibody fragment will depend, in part, on factors such as net charge, hydrophobicity, hydrophilicity etc., and will be apparent to those having skill in the art. For affinity chromatography purification an antibody, ligand, receptor or antigen can be used to which the antibody or antibody fragment binds. For example, for affinity chromatography purification of antibody or antibody fragment according to the present disclosure, a matrix with protein A or protein G may be used. The purity of an antibody or antibody fragment can be determined by any of a variety of well-known analytical methods including gel electrophoresis, high-pressure liquid chromatography, and the like.

### Specificity

In an embodiment, the present disclosure relates to a humanized antibody or antibody fragment specific for human CD117. In an embodiment, the present disclosure relates to a humanized antibody or antibody fragment specific for human CD117 disclosed in Table 18. In an embodiment, the present disclosure relates to a humanized antibody or antibody fragment specific for a polypeptide encoded by the amino acid sequence of SEQ ID NO: 1. In an embodiment, the present disclosure relates to a humanized antibody or antibody fragment specific for a polypeptide comprising the amino acid sequence of SEQ ID NO: 1.

In an embodiment, the present disclosure relates to a humanized antibody or antibody fragment that is specific for human CD117 and cynomolgus CD117.

In an embodiment, the present disclosure relates to a humanized antibody or antibody fragment specific for CD117 as disclosed herein, wherein said antibody or antibody fragment cross-competes with antibody SR-1 for binding to CD117. In another embodiment, the present disclosure relates to a humanized antibody or antibody fragment specific for CD117 as disclosed herein, wherein said antibody or antibody fragment cross-competes with an antibody or antibody fragment comprising a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9 for binding to CD117.

In an embodiment, the present disclosure relates to a humanized antibody or antibody fragment specific for CD117 as disclosed herein, wherein said antibody or antibody fragment binds to the same epitope on CD117 as antibody SR-1. In another embodiment, the present disclosure relates to a humanized antibody or antibody fragment specific for CD117 as disclosed herein, wherein said antibody or antibody fragment binds to the same epitope on CD117 as an antibody or antibody fragment comprising a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9.

In an embodiment, the present disclosure relates to a humanized antibody or antibody fragment specific for CD117 as disclosed herein, wherein said antibody or antibody fragment retains the binding specificity of antibody SR-1. In another embodiment, the present disclosure relates to a humanized antibody or antibody fragment specific for CD117 as disclosed herein, wherein said antibody or antibody fragment retains the binding specificity of an antibody or antibody fragment comprising a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9.

In an embodiment, the present disclosure relates to a humanized antibody or antibody fragment specific for CD117 as disclosed herein, wherein said antibody or antibody fragment binds to CD117 with about the same affinity as antibody SR-1. In another embodiment, the present disclosure relates to a humanized antibody or antibody fragment specific for CD117 as disclosed herein, wherein said antibody or antibody fragment binds to CD117 with about the same affinity as an antibody or antibody fragment comprising a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9.

In an embodiment, the present disclosure relates to a humanized antibody or antibody fragment specific for CD117 as disclosed herein, wherein said antibody or antibody fragment binds to CD117 with the same affinity as antibody SR-1. In another embodiment, the present disclosure relates to a humanized antibody or antibody fragment specific for CD117 as disclosed herein, wherein said antibody or antibody fragment binds to CD117 with the same affinity as an antibody or antibody fragment comprising a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9.

In an embodiment, the present disclosure relates to a humanized antibody or antibody fragment specific for CD117 as disclosed herein, wherein said antibody or antibody fragment binds to CD117 with at least the same affinity as antibody SR-1. In another embodiment, the present disclosure relates to a humanized antibody or antibody fragment specific for CD117 as disclosed herein, wherein said antibody or antibody fragment binds to CD117 with at least the same affinity as an antibody or antibody fragment comprising a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9.

In an embodiment, the present disclosure relates to a humanized antibody or antibody fragment specific for CD117 as disclosed herein, wherein said antibody or antibody fragment binds to CD117 with a higher affinity than antibody SR-1. In another embodiment, the present disclosure relates to a humanized antibody or antibody fragment specific for CD117 as disclosed herein, wherein said antibody or antibody fragment binds to CD117 with a higher affinity than an antibody or antibody fragment comprising a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9.

In an embodiment, the present disclosure relates to a humanized antibody or antibody fragment specific for CD117 as disclosed herein, wherein said antibody or antibody fragment binds to CD117 with a higher affinity than antibody SR-1. In another embodiment, the present disclosure relates to a humanized antibody or antibody fragment specific for CD117 as disclosed herein, wherein said antibody or antibody fragment binds to CD117 with a higher affinity than an antibody or antibody fragment comprising a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9.

In an embodiment, the present disclosure relates to a humanized antibody or antibody fragment specific for CD117 as disclosed herein, wherein said antibody or antibody fragment binds to CD117 with an at least two-fold higher affinity than an antibody or antibody fragment comprising a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9, and wherein the humanized antibody or antibody fragment comprises the two following mutations:
a) the asparagine in the LCDR1 region (SEQ ID NO: 7) is replaced by glutamic acid, histidine, glutamine or serine, and
b) the aspartic acid in the LCDR3 region (SEQ ID NO: 9) is replaced by glutamic acid. In certain embodiments said antibody or antibody fragment binds to CD117 with an at three-fold, an at least five-fold or an at least ten-fold higher affinity to CD117.

In an embodiment, the present disclosure relates to a humanized antibody or antibody fragment specific for CD117 as disclosed herein, wherein said antibody or antibody fragment has a lower number of critical deamidation sequence motifs than antibody SR-1. In another embodiment, the present disclosure relates to a humanized antibody or antibody fragment specific for CD117 as disclosed herein, wherein said antibody or antibody fragment has a lower number of critical deamidation sequence motifs than an antibody or antibody fragment comprising a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9.

In an embodiment, the present disclosure relates to a humanized antibody or antibody fragment specific for CD117 as disclosed herein, wherein said antibody or antibody fragment has a lower number of critical sequence motifs for chemical cleavage than antibody SR-1. In another embodiment, the present disclosure relates to a humanized antibody or antibody fragment specific for CD117 as disclosed herein, wherein said antibody or antibody fragment has a lower number of critical sequence motifs for chemical cleavage than an antibody or antibody fragment comprising a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9.

### Biological Function

In an embodiment, the present disclosure relates to a humanized antibody or antibody fragment specific for CD117 as disclosed herein, wherein said antibody or antibody fragment is functionally equivalent to antibody SR-1. In another embodiment, the present disclosure relates to a humanized antibody or antibody fragment specific for CD117 as disclosed herein, wherein said antibody or antibody fragment is functionally equivalent to an antibody or antibody fragment comprising a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9.

In an embodiment, the present disclosure relates to a humanized antibody or antibody fragment specific for CD117 as disclosed herein, wherein said antibody or antibody fragment inhibits the biological function of CD117 to at least the same degree as antibody SR-1. In another embodiment, the present disclosure relates to a humanized antibody or antibody fragment specific for CD117 as disclosed herein, wherein said antibody or antibody fragment inhibits the biological function of CD117 to at least to the same degree as an antibody or antibody fragment comprising a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9.

In certain embodiments, the present disclosure relates to humanized antibody or antibody fragment, wherein said humanized antibody or antibody fragment inhibits SCF binding to CD117. SCF binding to CD117 is important for the transmission of functional signals by CD117. Binding of CD117 to SCF can be measured by commonly known systems, such as the Octet system, or as described in the experimental section herein below. In an embodiment, the present disclosure relates to a humanized antibody or antibody fragment specific for CD117 as disclosed herein, wherein said antibody or antibody fragment inhibits SCF binding to CD117 to at least to the same degree as an antibody or antibody fragment comprising a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9.

In another embodiment, the present disclosure relates to a humanized antibody or antibody fragment specific for CD117 as disclosed herein, wherein said antibody or antibody fragment inhibits SCF binding to CD117 stronger than an antibody or antibody fragment comprising a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9.

In certain embodiments, the present disclosure relates to humanized antibody or antibody fragment, wherein said humanized antibody or antibody fragment inhibits SCF-dependent proliferation of CD117-positive cells. SCF-dependent proliferation can be tested by the skilled person by any commonly used assay, such as cell count, colony-forming unit (CFU) assay, cell titer glow (CTG; Promega) and carboxyfluorescein succinimidyl ester (CFSE) cell proliferation assay, or as described in the experimental section herein below. In an embodiment, the present disclosure relates to a humanized antibody or antibody fragment specific for CD117 as disclosed herein, wherein said antibody or antibody fragment inhibits SCF-dependent proliferation of CD117-positive cells to at least to the same degree as an antibody or antibody fragment comprising a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9.

In another embodiment, the present disclosure relates to a humanized antibody or antibody fragment specific for CD117 as disclosed herein, wherein said antibody or antibody fragment inhibits SCF-dependent proliferation of CD117-positive cells with an IC50 lower than an antibody or antibody fragment comprising a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9. In certain embodiments, said IC50 is at least 2-fold, at least 3-fold, at least 4-fold or at least 5-fold lower than that of an antibody or antibody fragment comprising a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9.

In certain embodiments, the present disclosure relates to a humanized antibody or antibody fragment, wherein said humanized antibody or antibody fragment inhibits SCF-dependent phosphorylation of CD117. SCF-dependent phosphorylation is another function of CD117. SCF-dependent phosphorylation can for example be measured in cells, e.g. TF-1 cells, expressing CD117, for example as described in the experimental section herein below. In an embodiment, the present disclosure relates to a humanized antibody or antibody fragment specific for CD117 as disclosed herein, wherein said antibody or antibody fragment inhibits SCF-dependent phosphorylation of CD117 to at least to the same degree as an antibody or antibody fragment comprising a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9.

In other embodiments, the present disclosure relates to a humanized antibody or antibody fragment specific for CD117 as disclosed herein, wherein said antibody or antibody fragment inhibits SCF-dependent phosphorylation of CD117 stronger than an antibody or antibody fragment comprising a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO:8 and the LCDR3 region of SEQ ID NO: 9.

### Effector Function

The Fc region of an immunoglobulin generally confers to the favorable pharmacokinetic properties of antibodies, such as prolonged half-life in serum and to the ability to induce effector function via binding to Fc receptors expressed on cells. On the other hand, binding to Fc receptors might also results in an undesirable activation of certain cell surface receptors leading to unwanted cytokine release and severe side effects upon systemic administration.

Accordingly, for certain therapeutic situations, it is desirable to reduce or abolish the normal binding of the wild-type Fc region of an antibody, such as of an wild-type IgG Fc region to one or more or all of Fc receptors and/or binding to a complement component, such as C1q in order to reduce or abolish the ability of the antibody to induce effector function. For instance, it may be desirable to reduce or abolish the binding of the Fc region of an antibody to one or more or all of the Fcy receptors, such as: FcγRI, FcγRIIa, FcγRIIb, FcγRIIIa. Effector function can include, but is not limited to, one or more of the following: complement dependent cytotoxicity (CDC), antibody-dependent cell-mediated cytotoxicity (ADCC), antibody-dependent cellular phagocytosis (ADCP), cytokine secretion, immune complex-mediated antigen uptake by antigen-presenting cells, binding to NK cells, binding to macrophages, binding to monocytes, binding to polymorphonuclear cells, direct signaling inducing apoptosis, crosslinking of target-bound antibodies, dendritic cell maturation, or T cell priming.

A reduced or abolished binding of an Fc region to an Fc receptor and/or to C1q is typically achieved by mutating a wild-type Fc region, such as of an IgG1 Fc region, more particular a human IgG1 Fc region, resulting in a variant or engineered Fc region of said wild-type Fc region, e.g., a variant human IgG1 Fc region. Substitutions that result in reduced binding can be useful. For reducing or abolishing the binding properties of an Fc region to an Fc receptor, non-conservative amino acid substitutions, i.e., replacing one amino acid with another amino acid having different structural and/or chemical properties, are preferred.

Accordingly, in an embodiment, the isolated antibody or antibody fragment specific for human CD117 according to the present disclosure comprises a variant Fc region having a reduced or abolished binding to an Fc receptor and/or to C1q when compared to the wild-type Fc region. In one such embodiment, the isolated antibody or antibody fragment according to the present disclosure comprises a variant Fc region that reduces or abolishes the ability of the antibody to induce effector function. In a further embodiment, the isolated antibody or antibody fragment according to the present disclosure does not substantially induce effector function.

In certain embodiments, the effector function is one or more selected from the group consisting of CDC, ADCC and ADCP. In an embodiment, the effector function is ADCC. In an embodiment, the effector function is CDC. In an embodiment, the effector function is ADCP. In an embodiment, the isolated antibody or antibody fragment according to the present disclosure does not substantially induce ADCC and/or CDC and/or ADCP. In an embodiment, the isolated antibody or antibody fragment according to the present disclosure does not induce ADCC or ADCP in vitro.

In an embodiment, the variant Fc region of the isolated antibody or antibody fragment according to the present disclosure comprises one or more amino acid substitutions that reduce or abolish the binding of the variant Fc region to one or more Fc receptors and/or to C1 q when compared to the wild-type Fc region. In an embodiment, the variant Fc region of the isolated antibody or antibody fragment according to the present disclosure comprises one or more amino acid substitutions that reduce or abolish the ability of the antibody to induce effector function when compared to the wild-type Fc region. In a particular embodiment, the one or more amino acid substitutions may reduce the binding affinity of the variant Fc region for one or more Fc receptors and/or to C1q by at least 2-fold, at least 5-fold, at least 10-fold, at least 20-fold or even at least 50-fold when compared to the wild-type Fc region. In alternative embodiments, the one or more amino acid substitutions may reduce the ability of the isolated antibody or antibody fragment according to the present disclosure to induce effector function by at least 2-fold, at least 5-fold, at least 10-fold, at least 20-fold or even at least 50-fold when compared to the wild-type Fc region.

In an embodiment, the variant Fc region of the isolated antibody or antibody fragment according to the present disclosure does not substantially bind to one or more Fc receptors and/or C1q. In an embodiment, the variant Fc region of the antibody according to the present disclosure does substantially abolish the ability of said antibody to induce effector function. In an embodiment, the antibody or antibody fragment according to the present disclosure does not substantially induce effector function. In an embodiment, said effect function is ADCC and/or ADCP and/or CDC. In an embodiment, the antibody or antibody fragment according to the present disclosure does not substantially induce effector function meaning that the level of induced effector function is not significantly above the background as measured in the absence of said antibody.

In an embodiment, the Fc receptor is a human Fc receptor. In an embodiment, the Fc receptor is an Fcy receptor. In an embodiment, the Fc receptor is a human FcγRIIIa, FcγRI, FcγRIIa and/or FcγRIIb.

In an embodiment, the isolated antibody or antibody fragment according to the present disclosure comprises a variant human IgG1 Fc region, which comprises one or more amino acid substitutions compared to the wild-type human IgG1 Fc region. In an embodiment, that one or more amino acid substitutions reduce or abolish the binding of the variant Fc region to an Fc receptor and/or to C1q and/or reduces the ability of said antibody to induce effector function when compared to the wild-type Fc region.

Various Fc modifications are commonly used. For a review see for example Antibodies (2020) 9: 64. Silencing functions include (numbering according EU index) the LALA (L234A/L235A), the PA-LALA (L234A/L235A/P329A) and the PG-LALA (L234A/L235A/P329G) mutations, as well as the AEASS mutations (L234A/L235E/G237A/A330S/P331S).

In certain embodiments, the present disclosure relates to humanized antibodies that are specific for CD117, wherein said humanized antibodies have a silenced Fc region. In certain embodiments, the present disclosure relates to humanized antibodies that are specific for CD117, wherein said humanized antibodies or antibody fragments are in the AEASS format.

In certain embodiments, the present disclosure relates to humanized antibodies that are specific for CD117, wherein said humanized antibodies comprise a Fc region in which at least the following modifications are present: L234A, L235E, G237A, A330S, P331S (numbering according EU index). In certain embodiments, the present disclosure relates to humanized antibodies that are specific for CD117, wherein said humanized antibodies comprise a Fc region in which the following modifications are present: L234A, L235E, G237A, A330S, P331S (numbering according EU index).

In certain embodiments, the present disclosure relates to humanized antibodies that are specific for CD117, wherein said humanized antibodies or antibody fragments are in the PA-LALA format. In certain embodiments, the present disclosure relates to humanized antibodies that are specific for CD117, wherein said humanized antibodies comprise a Fc region in which at least the following modifications are present: L234A, L235A, P329A (numbering according EU index). In certain embodiments, the present disclosure relates to humanized antibodies that are specific for CD117, wherein said humanized antibodies comprise a Fc region in which the following modifications are present: L234A, L235A, P329A (numbering according EU index).

In certain embodiments, the present disclosure relates to humanized antibodies that are specific for CD117, wherein said humanized antibodies or antibody fragments are in the PG-LALA format. In certain embodiments, the present disclosure relates to humanized antibodies that are specific for CD117, wherein said humanized antibodies comprise a Fc region in which at least the following modifications are present: L234A, L235A, P329G (numbering according EU index). In certain embodiments, the present disclosure relates to humanized antibodies that are specific for CD117, wherein said humanized antibodies comprise a Fc region in which the following modifications are present: L234A, L235A, P329G (numbering according EU index).

In certain embodiments, the present disclosure relates to a humanized antibody, wherein said humanized antibody comprises:
a) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 95,
b) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 81, the LCDR2 region of SEQ ID NO: 8 and the LCDR3 region of SEQ ID NO: 113,
c) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 113,
d) a variable heavy chain comprising a HCDR1 region of SEQ ID NO: 4, a HCDR2 region of SEQ ID NO: 71, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 95,
e) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 115,
f) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO:8 and the LCDR3 region of SEQ ID NO: 115,
g) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 121 and the LCDR3 region of SEQ ID NO:115,
h) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 123 and the LCDR3 region of SEQ ID NO: 115,
i) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 125 and the LCDR3 region of SEQ ID NO: 115,
j) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 127 and the LCDR3 region of SEQ ID NO: 115,
k) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 113,
l) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 81, the LCDR2 region of SEQ ID NO: 8 and the LCDR3 region of SEQ ID NO: 95,
m) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 9, or
n) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 81, the LCDR2 region of SEQ ID NO: 8 and the LCDR3 region of SEQ ID NO: 9,
wherein the Fc region of said humanized antibodies is silenced.

In certain embodiments, the present disclosure relates to a humanized antibody, wherein said humanized antibody comprises:
a) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 95,
b) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 81, the LCDR2 region of SEQ ID NO: 8 and the LCDR3 region of SEQ ID NO: 113,
c) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 113,
d) a variable heavy chain comprising a HCDR1 region of SEQ ID NO: 4, a HCDR2 region of SEQ ID NO: 71, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 95,
e) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 115,
f) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 115,
g) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO:121 and the LCDR3 region of SEQ ID NO: 115,
h) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 123 and the LCDR3 region of SEQ ID NO: 115,
i) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 125 and the LCDR3 region of SEQ ID NO: 115,
j) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO:127 and the LCDR3 region of SEQ ID NO: 115,
k) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO:8 and the LCDR3 region of SEQ ID NO: 113,
l) a variable heavy chain comprising a HCDR1 region of SEQ ID NO: 4, a HCDR2 region of SEQ ID NO: 5, a HCDR3 region of SEQ ID NO: 6 and a variable tight chain comprising, the LCDR1 region of SEQ ID NO: 81, the LCDR2 region of SEQ ID NO: 8 and the LCDR3 region of SEQ ID NO: 95,
m) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 9, or
n) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 81, the LCDR2 region of SEQ ID NO: 8 and the LCDR3 region of SEQ ID NO: 9,
wherein the Fc region of said humanized antibodies comprises the modifications L234A, L235E, G237A, A330S and P331S (numbering according EU index).

In certain embodiments, the present disclosure relates to a humanized antibody, wherein said humanized antibody comprises:
a) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 95,
b) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 81, the LCDR2 region of SEQ ID NO: 8 and the LCDR3 region of SEQ ID NO: 113,
c) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 113,
d) a variable heavy chain comprising a HCDR1 region of SEQ ID NO: 4, a HCDR2 region of SEQ ID NO: 71, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 95,
e) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 115,
f) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO:115,
g) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 121 and the LCDR3 region of SEQ ID NO: 115,
h) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 123 and the LCDR3 region of SEQ ID NO: 115,
i) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 125 and the LCDR3 region of SEQ ID NO: 115,
j) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 127 and the LCDR3 region of SEQ ID NO:115,
k) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 113,
l) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 81, the LCDR2 region of SEQ ID NO: 8 and the LCDR3 region of SEQ ID NO: 95,
m) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 9, or
n) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 81, the LCDR2 region of SEQ ID NO: 8 and the LCDR3 region of SEQ ID NO: 9,
wherein the Fc region of said humanized antibodies comprises the modifications L234A, L235A and P329A (numbering according EU index).

In certain embodiments, the present disclosure relates to a humanized antibody, wherein said humanized antibody comprises:
a) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 95,
b) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 81, the LCDR2 region of SEQ ID NO: 8 and the LCDR3 region of SEQ ID NO: 113,
c) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 113,
d) a variable heavy chain comprising a HCDR1 region of SEQ ID NO: 4, a HCDR2 region of SEQ ID NO: 71, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 95,
e) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 115,
f) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 115,
g) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 121 and the LCDR3 region of SEQ ID NO: 115,
h) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 123 and the LCDR3 region of SEQ ID NO: 115,
i) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 125 and the LCDR3 region of SEQ ID NO:115,
j) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 127 and the LCDR3 region of SEQ ID NO: 115,
k) a variable heavy chain comprising a HCDR1 region of SEQ ID NO: 4, a HCDR2 region of SEQ ID NO: 107, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 113,
l) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 81, the LCDR2 region of SEQ ID NO: 8 and the LCDR3 region of SEQ ID NO: 95,
m) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 9, or
n) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 81, the LCDR2 region of SEQ ID NO: 8 and the LCDR3 region of SEQ ID NO: 9,
wherein the Fc region of said humanized antibodies comprises the modifications L234A, L235A and P329G (numbering according EU index).

In certain embodiments, the present disclosure relates to a humanized antibody, wherein said humanized antibody comprises:
a) a variable heavy chain of SEQ ID NO: 22 and a variable light chain of SEQ ID NO: 98,
b) a variable heavy chain of SEQ ID NO: 22 and a variable light chain of SEQ ID NO: 99,
c) a variable heavy chain of SEQ ID NO: 22 and a variable light chain of SEQ ID NO: 100,
d) a variable heavy chain of SEQ ID NO: 70 and a variable light chain of SEQ ID NO: 98,
e) a variable heavy chain of SEQ ID NO: 22 and a variable light chain of SEQ ID NO: 105,
f) a variable heavy chain of SEQ ID NO: 106 and a variable light chain of SEQ ID NO: 105,
g) a variable heavy chain of SEQ ID NO: 106 and a variable light chain of SEQ ID NO: 117,
h) a variable heavy chain of SEQ ID NO: 106 and a variable light chain of SEQ ID NO: 118,
i) a variable heavy chain of SEQ ID NO: 106 and a variable light chain of SEQ ID NO: 119,
j) a variable heavy chain of SEQ ID NO: 106 and a variable light chain of SEQ ID NO: 120,
k) a variable heavy chain of SEQ ID NO: 106 and a variable light chain of SEQ ID NO: 100,
l) a variable heavy chain of SEQ ID NO: 22 and a variable light chain of SEQ ID NO: 97,
m) a variable heavy chain of SEQ ID NO: 22 and a variable light chain of SEQ ID NO: 61, or
n) a variable heavy chain of SEQ ID NO: 22 and a variable light chain of SEQ ID NO: 62,
wherein the Fc region of said humanized antibodies is silenced.

In certain embodiments, the present disclosure relates to a humanized antibody, wherein said humanized antibody comprises
a) a variable heavy chain of SEQ ID NO: 22 and a variable light chain of SEQ ID NO: 98,
b) a variable heavy chain of SEQ ID NO: 22 and a variable light chain of SEQ ID NO: 99,
c) a variable heavy chain of SEQ ID NO: 22 and a variable light chain of SEQ ID NO: 100,
d) a variable heavy chain of SEQ ID NO: 70 and a variable light chain of SEQ ID NO: 98,
e) a variable heavy chain of SEQ ID NO: 22 and a variable light chain of SEQ ID NO: 105,
f) a variable heavy chain of SEQ ID NO: 106 and a variable light chain of SEQ ID NO: 105,
g) a variable heavy chain of SEQ ID NO: 106 and a variable light chain of SEQ ID NO: 117,
h) a variable heavy chain of SEQ ID NO: 106 and a variable light chain of SEQ ID NO: 118,
i) a variable heavy chain of SEQ ID NO: 106 and a variable light chain of SEQ ID NO: 119,
j) a variable heavy chain of SEQ ID NO: 106 and a variable light chain of SEQ ID NO: 120,
k) a variable heavy chain of SEQ ID NO: 106 and a variable light chain of SEQ ID NO: 100,
1) a variable heavy chain of SEQ ID NO: 22 and a variable light chain of SEQ ID NO: 97,
m) a variable heavy chain of SEQ ID NO: 22 and a variable light chain of SEQ ID NO: 61, or
n) a variable heavy chain of SEQ ID NO: 22 and a variable light chain of SEQ ID NO: 62,
wherein the Fc region of said humanized antibodies comprises the modifications L234A, L235E, G237A, A330S and P331S (numbering according EU index).

In certain embodiments, the present disclosure relates to a humanized antibody, wherein said humanized antibody comprises:
a) a variable heavy chain of SEQ ID NO: 22 and a variable light chain of SEQ ID NO: 98,
b) a variable heavy chain of SEQ ID NO: 22 and a variable light chain of SEQ ID NO: 99,
c) a variable heavy chain of SEQ ID NO: 22 and a variable light chain of SEQ ID NO: 100,
d) a variable heavy chain of SEQ ID NO: 70 and a variable light chain of SEQ ID NO: 98,
e) a variable heavy chain of SEQ ID NO: 22 and a variable light chain of SEQ ID NO: 105,
f) a variable heavy chain of SEQ ID NO: 106 and a variable light chain of SEQ ID NO: 105,
g) a variable heavy chain of SEQ ID NO: 106 and a variable light chain of SEQ ID NO: 117,
h) a variable heavy chain of SEQ ID NO: 106 and a variable light chain of SEQ ID NO: 118,
i) a variable heavy chain of SEQ ID NO: 106 and a variable light chain of SEQ ID NO: 119,
j) a variable heavy chain of SEQ ID NO: 106 and a variable light chain of SEQ ID NO: 120,
k) a variable heavy chain of SEQ ID NO: 106 and a variable light chain of SEQ ID NO: 100,
l) a variable heavy chain of SEQ ID NO: 22 and a variable light chain of SEQ ID NO: 97,
m) a variable heavy chain of SEQ ID NO: 22 and a variable light chain of SEQ ID NO: 61, or
n) a variable heavy chain of SEQ ID NO: 22 and a variable light chain of SEQ ID NO: 62,
wherein the Fc region of said humanized antibodies comprises the modifications L234A, L235A and P329A (numbering according EU index).

In certain embodiments, the present disclosure relates to a humanized antibody, wherein said humanized antibody comprises:
a) a variable heavy chain of SEQ ID NO: 22 and a variable light chain of SEQ ID NO: 98,
b) a variable heavy chain of SEQ ID NO: 22 and a variable light chain of SEQ ID NO: 99,
c) a variable heavy chain of SEQ ID NO: 22 and a variable light chain of SEQ ID NO: 100,
d) a variable heavy chain of SEQ ID NO: 70 and a variable light chain of SEQ ID NO: 98,
e) a variable heavy chain of SEQ ID NO: 22 and a variable light chain of SEQ ID NO: 105,
f) a variable heavy chain of SEQ ID NO: 106 and a variable light chain of SEQ ID NO: 105,
g) a variable heavy chain of SEQ ID NO: 106 and a variable light chain of SEQ ID NO: 117,
h) a variable heavy chain of SEQ ID NO: 106 and a variable light chain of SEQ ID NO: 118,
i) a variable heavy chain of SEQ ID NO: 106 and a variable light chain of SEQ ID NO: 119,
j) a variable heavy chain of SEQ ID NO: 106 and a variable light chain of SEQ ID NO: 120,
k) a variable heavy chain of SEQ ID NO: 106 and a variable light chain of SEQ ID NO: 100,
l) a variable heavy chain of SEQ ID NO: 22 and a variable light chain of SEQ ID NO: 97,
m) a variable heavy chain of SEQ ID NO: 22 and a variable light chain of SEQ ID NO: 61, or
n) a variable heavy chain of SEQ ID NO: 22 and a variable light chain of SEQ ID NO: 62,
wherein the Fc region of said humanized antibodies comprises the modifications L234A, L235A and P329G (numbering according EU index).

### Therapeutic Methods

The antibodies and antibody fragments of the present disclosure, or pharmaceutical compositions incorporating the same, can be used for the treatment of various conditions. For example, such antibodies can be used in ablation of endogenous hematopoietic stem and progenitor cells (HSPCs) in a subject in need thereof. Ablation of endogenous HSPCs is an initial step in stem cell replacement therapy. Stem cell replacement therapy generally involves reducing or eliminating endogenous HSPCs, which are defective in some respect, and replacing them with replacement HSPCs. The replacement HSPCs can be autologous, allogenic or xenogenic. Endogenous HSPCs may be defective as a result of hereditary mutation impairing function or expression (e.g., sickle cell anemia or thalassemia), as a result of a hematologic cancer, or as a result of damage from chemotherapy used in treating a cancer. Endogenous HSPCs may also be replaced in conjunction with an organ transplant because the endogenous HSPCs would result in immune attack of the transplant.

Antibodies against CD117 can also be used in treatment of cancers expressing CD117. Such cancers include hematological cancers, such as AML and solid tumors, such as mast cell cancer, testicular stromal cancer, gastrointestinal stromal cancer, melanoma, breast and lung cancer. Expression of CD117 is preferably at a higher level than tissue matched normal control cells as determined by immunohistochemistry assay.

The isolated antibody or antibody fragment according to the present disclosure may also be used in other therapeutic methods, such as the treatment of inflammatory diseases, autoimmune diseases, urticaria, prurigo nodularis, eosinophilic esophagitis, mastocytosis, hematologic malignancies and potentially other diseases.

In certain embodiments, the present disclosure relates to the use of the humanized antibodies or antibody fragments of the present disclosure for use in medicine. In other embodiments, the present disclosure relates to the use of the humanized antibodies or antibody fragments of the present disclosure for use in the treatment of cancer. In other embodiments, the present disclosure relates to the use of the humanized antibodies or antibody fragments of the present disclosure for use in the treatment of AML. In other embodiments, the present disclosure relates to the use of the humanized antibodies or antibody fragments of the present disclosure for use in the treatment of a solid cancer. In certain embodiments said solid cancer is mast cell cancer, testicular stromal cancer, gastrointestinal stromal cancer, melanoma, breast or lung cancer.

In certain embodiments, the present disclosure relates to the use of the humanized antibodies or antibody fragments of the present disclosure for use in the treatment of a disease associated with the undesired presence of CD117. In certain embodiments, the present disclosure relates to the use of the humanized antibodies or antibody fragments of the present disclosure for use in the treatment of a disease associated with the undesired presence of CD117-positive cells.

In an embodiment, the disease to be treated is a proliferative disease. In a particular embodiment, the disease is cancer. Non-limiting examples of cancers include hematologic malignancies like chronic myelomonocytic leukemia (CMML), acute myeloid leukemia (AML), myelodysplastic syndrome, mastocytosis and non-hematologic malignancies like bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, sarcoma, skin cancer, squamous cell carcinoma, bone cancer, melanoma, renal cell carcinoma, and kidney cancer.

In an embodiment, the present disclosure provides a method for the treatment of a disease.

In an embodiment, the present disclosure provides a method for the treatment of a disease comprising administering to a patient an antibody or antibody fragment of the present disclosure.

In an embodiment, the present disclosure provides a method for the treatment of a disease comprising administering to a subject in need there of an antibody or antibody fragment of the present disclosure.

In an embodiment, the present disclosure provides a method for the prevention of a disease.

In an embodiment, the present disclosure provides a method for the prevention of a disease comprising administering to a subject an antibody or antibody fragment of the present disclosure.

In an embodiment, the present disclosure provides an isolated antibody or antibody fragment according to the present disclosure for the treatment of a disease. In an embodiment, the present disclosure provides an isolated antibody or antibody fragment according to the present disclosure for use in the treatment of a disease. In an embodiment, the present disclosure provides an isolated antibody or antibody fragment according to the present disclosure for use in the treatment of a disease in a subject in need thereof.

In an embodiment, the present disclosure provides the use of an isolated antibody or antibody fragment according to the present disclosure for the manufacture of a medicament. In an embodiment, the present disclosure provides an isolated antibody or antibody fragment according to the present disclosure for use as a medicament. In an embodiment, the present disclosure provides an isolated antibody or antibody fragment according to the present disclosure for use in medicine. In an embodiment, the present disclosure provides an isolated antibody or antibody fragment according to the present disclosure for use as a medicament for the treatment of a subject in need thereof.

In an embodiment, the present disclosure provides an isolated antibody or antibody fragment specific for human CD117 according to the present disclosure for use in a method of treating a subject having a disease comprising administering to the subject a therapeutically effective amount of an antibody or antibody fragment according to the present disclosure.

In an embodiment, the method further comprises administering to the subject a therapeutically effective amount of at least one additional therapeutic agent. The subject in need of treatment is typically a mammal, more specifically a human. For use in therapeutic methods, an isolated antibody or antibody fragment according to the present disclosure would be formulated, dosed, and administered in a way consistent with good medical practice.

### Pharmaceutical Compositions

In an embodiment, the present disclosure provides a pharmaceutical composition comprising an isolated antibody or antibody fragment according to the present disclosure and a pharmaceutically acceptable carrier or excipient.

Antibodies are administered in an effective regime meaning a dosage, route of administration and frequency of administration that achieves the intended purpose, such as reduction of endogenous HSPCs or of cancer cells expressing CD117. In some instances, efficacy can be observed in an individual patient relative to historical controls or past experience in the same patient. In other instances, efficacy can be demonstrated in a preclinical or clinical trial in a population of treated patients relative to a control population of untreated patients.

The pharmaceutical compositions may further comprise at least one other pharmaceutically active compound. The pharmaceutical composition according to the present disclosure can be used in the diagnosis, prevention and/or treatment of diseases associated with the undesired presence of CD117, in particular human CD117. The pharmaceutical composition according to the present disclosure can be used in the diagnosis, prevention and/or treatment of diseases associated with the undesired presence of CD117-positive cells, in particular CD117-positive human cells. In particular, the present disclosure provides a pharmaceutical composition comprising an antibody or antibody fragment according to the present disclosure that is suitable for prophylactic, therapeutic and/or diagnostic use in a mammal, more particular in a human.

In general, an antibody or antibody fragment according to the present disclosure may be formulated as a pharmaceutical composition comprising at least one antibody or antibody fragment according to the present disclosure and at least one pharmaceutically acceptable carrier or excipient, and optionally one or more further pharmaceutically active compounds. Such a formulation may be suitable for oral, parenteral, topical administration or for administration by inhalation. Accordingly, a pharmaceutical composition comprising at least one antibody or antibody fragment according to the present disclosure may be administered parenterally, such as intravenously, or intramuscularly, or subcutaneously. Alternatively, an antibody of the invention may be administered via a non-parenteral route, such as per-orally or topically. In a preferred embodiment, a pharmaceutical composition comprising an antibody or antibody fragment according to the present disclosure is administered intravenously or subcutaneously.

In particular, an antibody or antibody fragment according to the present disclosure may be used in combination with one or more pharmaceutically active compounds that are or can be used for the prevention and/or treatment of the diseases in which a target antigen of interest is involved, as a result of which a synergistic effect may or may not be obtained. Examples of such compounds, as well as routes, methods and pharmaceutical formulations or compositions for administering them will be clear to the clinician.

In an embodiment, the present disclosure provides a pharmaceutical composition comprising an antibody or antibody fragment according to the present disclosure for use in the prevention and/or treatment of a disease associated with the undesired presence of CD117, in particular human CD117. In an embodiment, the present disclosure provides a pharmaceutical composition comprising an antibody or antibody fragment according to the present disclosure for use in the prevention and/or treatment of a disease associated with the undesired presence of CD117-positive cells, in particular CD117-positive human cells. In an embodiment, the present disclosure provides a pharmaceutical composition comprising an antibody or antibody fragment according to the present disclosure for the use as a medicament. In an embodiment, the present disclosure provides a pharmaceutical composition comprising an antibody or antibody fragment according to the present disclosure for use in the prevention and/or treatment of an autoimmune disease and/or inflammatory disease and/or cancer.

In an embodiment, the present disclosure provides a method for the treatment of an autoimmune disease and/or inflammatory disease and/or cancer in a subject in need thereof using a pharmaceutical composition comprising an antibody or antibody fragment according to the present disclosure.

Further provided is a method of producing an antibody or antibody fragment according to the present disclosure in a form suitable for administration in vivo, the method comprising (a) obtaining an antibody or antibody fragment by a method according to the present disclosure, and (b) formulating said antibody or antibody fragment with at least one pharmaceutically acceptable carrier or excipient, whereby a preparation of antibody or antibody fragment is formulated for administration in vivo. Pharmaceutical compositions according to the present disclosure comprise a therapeutically effective amount of one or more antibodies or antibody fragments according to the present disclosure dissolved in a pharmaceutically acceptable carrier or excipient.

### Diagnostic Use

In an embodiment, the present disclosure provides the use of an isolated antibody or antibody fragment specific for human CD117 according to the present disclosure for the diagnosis of a disease. In an embodiment, the present disclosure provides the use of an antibody or antibody fragment according to the present disclosure for the detection of CD117, in particular human CD117. In an embodiment, the present disclosure provides a method for detecting CD117 in a subject or a sample, comprising the step of contacting said subject or sample with an isolated antibody or antibody fragment specific for human CD117 of the present disclosure. In an embodiment, the present disclosure provides a method for diagnosing a disease in a subject, comprising the step of contacting said subject or sample with an isolated antibody or antibody fragment according to the present disclosure. The antibodies may also be used to determine CD117 expression levels in cells from patients. The CD117 expressions levels may serve as therapeutic biomarkers, for example for patient stratification.

### EXAMPLES

### Example 1: Anti-CD117 Antibodies of the Present Invention

Antibody SR-1 is a mouse antibody first described in Broudy et al, Blood (1992) 79: 338-46. The sequence of SR-1 is, for example, disclosed in US20200165337A1 (see SEQ ID No. 1 and 5 therein).

The sequence of the variable chains and the CDRs are shown in the following table:

TABLE 1
SR-1

| SEQ ID | | |
|---|---|---|
| No. | Comment | Sequence |
| 2 | VH | QVQLQQPGAELVKPGASVKMSCKASGYTFTSYNMHWVKQTPGQG |
| | | LEWIGVIYSGNGDTSYNQKFKGKATLTADKSSSTAYMQINSLTSEDSA |
| | | VYYCARERDTRFGNWGQGTLVTVSA |
| 3 | VL | NIVLTQSPASLAVSLGLRATISCRASESVDIYGNSFMHWYQQKPGQP |
| | | PKLLIYLASNLESGVPARFSGSGSRTDFTLTIDPVEADDAATYYCQQN |
| | | NEDPYTFGGGTKLEIK |
| 4 | HCDR1 (Kabat) | SYNMH |
| 5 | HCDR2 (Kabat) | VIYSGNGDTSYNQKFKG |
| 6 | HCDR3 (Kabat) | ERDTREGN |
| 7 | LCDR1 (Kabat) | RASESVDIYGNSFMH |
| 8 | LCDR2(Kabat) | LASNLES |
| 9 | LCDR3 (Kabat) | QQNNEDPYT |
| 10 | HCDR1 (IMGT) | GYTFTSYN |
| 11 | HCDR2 (IMGT) | IYSGNGDT |
| 12 | HCDR3 (IMGT) | ARERDTRFGN |
| 13 | LCDR1 (IMGT) | ESVDIYGNSF |
| 14 | LCDR2 (IMGT) | AS |
| 15 | LCDR3 (IMGT) | QQNNEDPYTF |

Humanization of SR-1 is described in WO2007/127317 and WO2020/112687. WO2007/127317 utilizes a classical humanization approach by CDR grafting to generate an antibody designated AMG191. No further engineering was performed on AMG191. Also WO2020/112687 utilized a relatively classical approach, with certain modifications being introduced in the framework regions. Such antibodies therefore still have shortcomings, such as the occurrence of unwanted post-translational modification sites or sites which render the humanized antibodies susceptible for fragmentation. Such sites occur within the CDRs of SR-1, and both mentioned humanization approaches did not aim to remove these sites, since it is expected that engineering within the CDR may affect the properties of the antibody, e.g., the affinity. By a sophisticated approach, the inventors of the present application succeeded to humanize SR-1, while at the same time unwanted post-translational modification and fragmentation sites were removed. Surprisingly the antibodies generated also did not compromise on the affinity and the activity of the antibodies. This was achieved by a stepwise approach as further detailed in the following examples.

### Example 2: Humanization of Murine Antibody SR-1

The humanization process relied on a combination of CDR-grafting technologies coupled with the latest research on antibody structure and up-date database of mature human IgG sequences. A number of human framework sequences were identified that were used as “acceptor” frameworks for the target CDR sequences. All acceptor sequences come from mature human IgG. As a result, the humanized sequences are expected not to be immunogenic and retain the canonical structure of the CDR loops.

### Example 2.1: Design of Humanized Variants of the Variable Heavy Chain

The closest human germline gene V-region that was identified in Homo sapiens was IGHV1-3*01:

(SEQ ID NO. 16)
QVQLVQSGAEVKKPGASVKVSCKASGYTFTSYAMHWVRQAPGQRLEWMG
WINAGNGNTKYSQKFQGRVTITRDTSASTAYMELSSLRSEDTAVYYCAR

Databases of human IgG sequences were searched for comparison to the murine VH domain using BLAST search algorithms, and candidate human variable domains were selected from the top 200 BLAST results. These were reduced to three candidates based on a combination of framework homology, maintaining key framework residues and canonical loop structure.

The three acceptor frameworks are:

>QEP24303:
(SEQ ID NO. 17)
QVQLVQSGAEVKKPGASVKVSCKASGYTFTTYGISWVRQAPGQGLEWMGWISTYNGDTNYAQ
KLRGRVTMTTDTSTSTAYMELRSLTSDDTAVYYCARDRLSTGTTFYYWGQGTLVTVSS
>ADWO8092:
(SEQ ID NO. 18)
QVQLVESGAEVKKPGASVKLSCKASGYTFSSYWMHWVRQAPGQRLEWMGEINPDNGHTNYN
EKFKSRVTITVDKSASTAYMELSSLRSEDTAVYYCAREADYSYGAFDIWGPGTTVTVSS
>AKU38660:
(SEQ ID NO. 19)
QVQLVQSGAEVRKPGASVKLSCKASGYTFTRYSMHWVRQAPGQGLEWMGIINPSGGSTSYAQ
NFQGRVTLTRDTSTSTVYMELSSLRSEDTAVYYCTRDRLRNWFDPWGQGTLVTVSS

Two additional frameworks were designed based on homology and sequences that would allow a greater sequence with the aim to achieve a more stable product:

>IGHV3-23*03:
(SEQ ID NO. 20)
EVQLLESGGGLVQPGGSLRLSCAASGFTFSSYAMSWVRQAPGKGLEWVSVIYSGGSSTYYADS
VKGRFTISRDNSKNTLYLQMNSLRAEDTAVYYCAK
>IGHV5-51*01:
(SEQ ID NO. 21)
EVQLVQSGAEVKKPGESLKISCKGSGYSFTSYWIGWVRQMPGKGLEWMGIIYPGDSDTRYSPS
FQGQVTISADKSISTAYLQWSSLKASDTAMYYCAR

With the CDRs of the murine VH of SR-1 grafted into aforementioned acceptor frameworks, the following humanized variants were selected and synthesized:

>VH1
(SEQ ID NO. 22)
QVQLVQSGAEVKKPGASVKVSCKASGYTFTSYNMHWVRQAPGQGLEWMGVIYSGNGDTSYN
QKFKGRVTMTTDTSTSTAYMELRSLTSDDTAVYYCARERDTRFGNWGQGTLVTVSA
>VH2
(SEQ ID NO. 23)
QVQLVESGAEVKKPGASVKLSCKASGYTFTSYNMHWVRQAPGQRLEWMGVIYSGNGDTSYNQ
KFKGRVTITVDKSASTAYMELSSLRSEDTAVYYCARERDTRFGNWGQGTLVTVSA
>VH3
(SEQ ID NO. 24)
QVQLVQSGAEVRKPGASVKLSCKASGYTFTSYNMHWVRQAPGQGLEWMGVIYSGNGDTSYN
QKFKGRVTLTRDTSTSTVYMELSSLRSEDTAVYYCARERDTRFGNWGQGTLVTVSA
>VH4
(SEQ ID NO. 25)
EVQLLESGGGLVKPGASLRLSCAASGYTFTSYNIHWVRQAPGQGLEWVSAIYSGNVDTSYSQKF
KGRFTISRDNSKNTLYLQMNSLRAEDTAVYYCARERDTRFGNWGQGTLVTVSA
>VH5
(SEQ ID NO. 26)
EVQLVQSGAEVKKPGESLKISCKGSGYTFTSYNMHWVRQMPGKGLEWMGVIYSGNADTSYNQ
KFKGQVTISADKSISTAYLQWSSLKASDTAMYYCARERDTRFGNWGQGTLVTVSA

SEQ ID No.s of the variable heavy chains including their CDR are shown in the following table.

TABLE 2

| | | HCDR1 | | HCDR2 | | HCDR3 | |
|---|---|---|---|---|---|---|---|
| | VH | Kabat | IMGT | Kabat | IMGT | Kabat | IMGT |
| SR-1 | 2 | 4 | 10 | 5 | 11 | 6 | 12 |
| VH1 | 22 | 4 | 10 | 5 | 11 | 6 | 12 |
| VH2 | 23 | 4 | 10 | 5 | 11 | 6 | 12 |
| VH3 | 24 | 4 | 10 | 5 | 11 | 6 | 12 |
| VH4 | 25 | 27 | 10 | 28 | 30 | 6 | 12 |
| VH5 | 26 | 4 | 10 | 29 | 31 | 6 | 12 |

SEQ ID No. 27:
SYNIH
SEQ ID No. 28:
AIYSGNVDTSYSQKFKG
SEQ ID No. 29:
VIYSGNADTSYNQKFKG
SEQ ID No. 30:
IYSGNVDT.
SEQ ID No. 31:
TYSGNADT

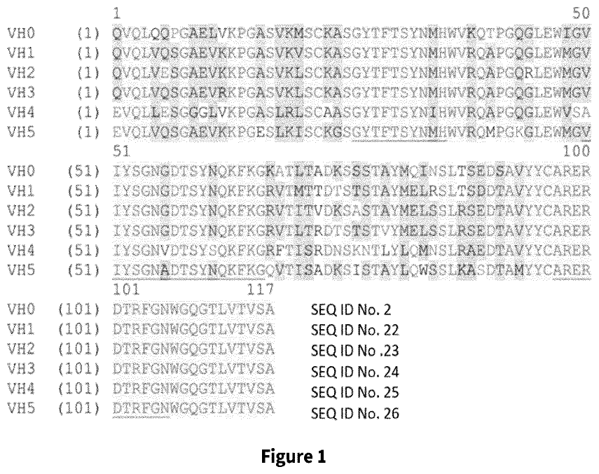

An alignment of the humanized variants to the original murine SR-1 sequence is shown in FIG. 1. The homology of the humanized variants to original murine VH sequence is shown in the following table.

TABLE 3

| | Identical amino acids | Consensus amino acids |
|---|---|---|
| VH1 | 83.8% | 92.3% |
| VH2 | 82.9% | 90.6% |
| VH3 | 83.8% | 90.6% |
| VH4 | 72.6% | 84.6% |
| VH5 | 76.9% | 88.0% |

### Example 2.2: Design of Humanized Variants of the Variable Light Chain

The closest human germline gene V-region that was identified in Homo sapiens was IGKV3D-11*02:

(SEQ ID NO. 32)
EIVLTQSPATLSLSPGERATLSCRASQSVSSYLAWYQQKPGQAPR
LLIYDASNRATGIPARFSGSGPGTDFTLTISSLEPEDFAVYYCQQ
RSNWH.

Databases of human IgK sequences were searched for comparison to the murine VL domain using BLAST search algorithms, and candidate human variable domains were selected from the top 200 BLAST results. These were reduced to two candidates based on a combination of framework homology, maintaining key framework residues and canonical loop structure.

The two acceptor frameworks are:

> ADU57829:
(SEQ ID NO. 33)
EIVLTQSPSSLSASVGDRVTITCRASQSISSYLNWYQQKPGKAPK
LLIYAASSLQSGVPSRFSGSGSGTDFTLTISSLQPEDFATYYCQQ
SYSTPYTFGQGTKLEIKS
> CAB46458:
(SEQ ID NO. 34)
DIVMTQSPSTLSASVGDRVTITCRASQSISSWLAWYQQKPGKAPK
LLIYEASSLESGVPSRFSGSGSGTEFTLTISSLQPDDFATYYCQQ
YNSYPYTFGQGTKLEIK

The latter framework (CAB46458) was used in two approaches—one with the framework as shown above (VL2) and one with an additional mutation introduced (VL3). Also three additional variants were chosen based on homology:

>IGKV3-15*01
(SEQ ID NO. 35)
EIVMTQSPATLSVSPGERATLSCRASQSVSSNLAWYQQKPGQAPR
LLIYGASTRATGIPARFSGSGSGTEFTLTISSLQSEDFAVYYCQQ
YNNWP
>IGKVI-39*01
(SEQ ID NO. 36)
DIQMTQSPSSLSASVGDRVTITCRASQSISSYLNWYQQKPGKAPK
LLIYAASSLQSGVPSRFSGSGSGTDFTLTISSLQPEDFATYYCQQ
SYSTP
>IGKV3D-11*01
(SEQ ID NO. 37)
EIVLTQSPATLSLSPGERATLSCRASQGVSSYLAWYQQKPGQAPR
LLIYDASNRATGIPARFSGSGPGTDFTLTISSLEPEDFAVYYCQQ
RSNWH

With the CDRs of the murine VL of SR-1 grafted into aforementioned acceptor frameworks, the following humanized variants were selected and synthesized:

>VL1
(SEQ ID NO. 38)
EIVLTQSPSSLSASVGDRVTITCRASESVDIYGNSFMHWYQQKPG
KAPKLLIYLASNLESGVPSRFSGSGSGTDFTLTISSLQPEDFATY
YCQQNNEDPYTFGQGTKLEIK
>VL2
(SEQ ID NO. 39)
DIVMTQSPSTLSASVGDRVTITCRASESVDIYGNSFMHWYQQKPG
KAPKLLIYLASNLESGVPSRFSGSGSGTEFTLTISSLQPDDFATY
YCQQNNEDPYTFGQGTKLEIK.
>VL3
(SEQ ID NO. 40)
DIVMTQSPSTLSASVGDRVTITCRASESVDIYGSSFMHWYQQKPG
QPPKLLIYLASNLESGVPSRFSGSGSGTEFTLTISSLEADDVATY
YCQQNNEDPYTFGGGTKLEIK
>VL4
(SEQ ID NO. 41)
EIVMTQSPATLSVSPGERATLSCRASESVDIYGQSFMHWYQQKPG
QAPRLLIYLASNLESGIPARFSGSGSGTEFTLTISSLQSEDFAVY
YCQQNDEEPYTFGGGTKLEIK
>VL5
(SEQ ID NO. 42)
DIQMTQSPSSLSASVGDRVTITCRASESVDIYGNAFMHWYQQKPG
KAPKLLIYLASNLESGVPSRFSGSGSGTDFTLTISSLQPEDFATY
YCQQANEEPYTFGGGTKLEIK
>VL6
(SEQ ID NO. 43)
EIVLTQSPATLSLSPGERATLSGRASESVDIYGQSFMHWYQQKPG
QAPRLLIYLASNLESGIPARFSGSGPGTDFTLTISSLEPEDFAVY
YCQQNNENPYTFGGGTKLEIK

SEQ ID No.s of the variable light chains including their CDR are shown in the following table.

TABLE 4

| | | LCDR1 | | LCDR2 | | LCDR3 | |
|---|---|---|---|---|---|---|---|
| | VL | Kabat | IMGT | Kabat | IMGT | Kabat | IMGT |
| SR-1 | 3 | 7 | 13 | 8 | 14 | 9 | 15 |
| VL1 | 38 | 7 | 13 | 8 | 14 | 9 | 15 |
| VL2 | 39 | 7 | 13 | 8 | 14 | 9 | 15 |
| VL3 | 40 | 44 | 47 | 8 | 14 | 9 | 15 |
| VL4 | 41 | 45 | 48 | 8 | 14 | 50 | 53 |
| VL5 | 42 | 46 | 49 | 8 | 14 | 51 | 54 |
| VL6 | 43 | 45 | 48 | 8 | 14 | 52 | 55 |

SEQ ID No. 44:
RASESVDIYGSSFMH
SEQ ID No. 45:
RASESVDIYGQSFMH
SEQ ID No. 46:
RASESVDIYGNAFMH
SEQ ID No. 47:
ESVDIYGSSF
SEQ ID No. 48:
ESVDIYGQSF
SEQ ID No. 49:
ESVDIYGNAF
SEQ ID No. 50:
QQNDEEPYT
SEQ ID No. 51:
QQANEEPYT
SEQ ID No. 52:
QQNNENPYT
SEQ ID No. 53:
QQNDEEPYTF
SEQ ID No. 54:
QQANEEPYTF
SEQ ID No. 55:
QQNNENPYTF

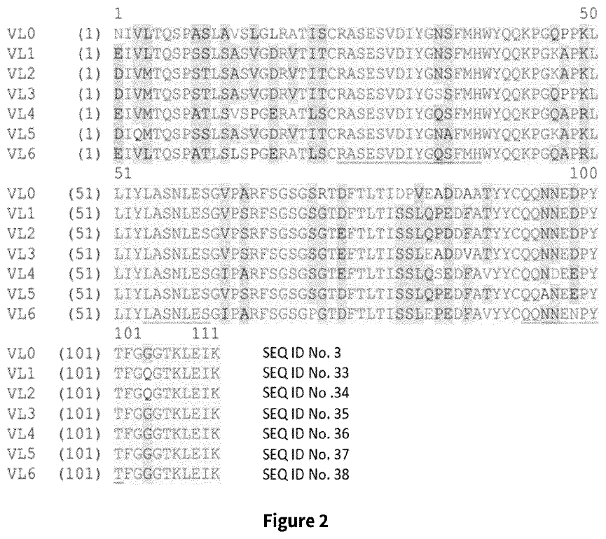

An alignment of the humanized variants to the original murine SR-1 sequence is shown in FIG. 2. The homology of the humanized variants to original murine VL sequence is shown in the following table.

TABLE 5

| | Identical amino acids | Consensus amino acids |
|---|---|---|
| VL1 | 82.0% | 88.3% |
| VL2 | 80.2% | 88.3% |
| VL3 | 83.8% | 91.9% |
| VL4 | 79.3% | 90.1% |
| VL5 | 78.4% | 87.4% |
| VL6 | 81.1% | 89.2% |

### Example 2.3: Expression and Purification of the Humanized Variants

DNA coding for the amino acid sequence of each antibody was synthesized and cloned into the mammalian transient expression plasmid pETE V2 (Fusion Antibodies, Northern Ireland)). Antibodies were expressed using a CHO based transient expression system and the resulting antibody containing cell culture supernatants were clarified by centrifugation and filtration. Antibodies were purified using a state-of-the-art chromatography equipment from cell culture supernatants via affinity chromatography. Purified antibodies were buffer exchanged into phosphate buffered saline solution. The purity of these antibodies was determined to be >95%, as judged by reducing and denaturing Sodium Dodecyl Sulfate Polyacrylamide gels. Antibody concentration was determined by measuring absorbance at 280 nm.

The five humanized variable heavy chains were tested in combination with five of the humanized variable light chains, leading to a total of twenty-five (25) combinations. Of these 25 combinations, nine could not be produced. These nine combinations include all combinations which contain either the humanized heavy chain VH4 or the humanized light chain VL6. The remaining 16 combinations could be produced in sufficient quantity for further characterization.

### Example 2.4: Affinity Determination of the Humanized Variants

IgG antibodies were immobilized on biosensors using suitable capture surfaces and binding of soluble antigen to immobilized antibodies was monitored by BLI (Octet). The resulting sensorgrams were analyzed using the supplied software (Fortebio). As antigen human recombinant CD117 (c-Kit) from Sino Biological was used (Catalog No. 11996-H08H, Lot #LC14SE0812). As a pre-experiment a series of experiments was performed to optimize assay parameters. The following parameters were selected:

TABLE 6

| Antibody loading concentration | 0.15 | ug/ml |
|---|---|---|
| Antigen stability @ 25° C., | >4 | hrs |
| shaking @1000 rpm | | |
| Running Buffer | HBS-P+ with | |
| | 10 uM DTT | |
| Time for association | 900 | sec |
| Time for dissociation | 900 | sec |

| | |
|---|---|
| Kinetic screening protocol required | Modified |
| Antigen screening range (seven | 100-0.1372 nM |
| concentrations, dilute 3-fold) | |
| Interaction/fitting model | 1:1 |

In the main experiment kinetic assays were performed by first capturing IgG using anti-human Fc biosensors. The mAb capture biosensors were then submerged in wells containing different concentrations of antigen (association stage) followed by a dissociation step in running buffer. To allow for double reference correction, IgG-captured sensors were dipped into wells containing only buffer and blank sensors were also dipped into wells containing the antigen. This referencing provided a means of compensating for both the natural dissociation of the capture IgG and non-specific binding of the antigen to the sensor surface. Steps were performed at 25° C. at a constant shaking of 1000 rpm. New sensors were used for each sample. Dissociation rate constants (Kd) were calculated using the ForteBio Data Analysis software. All consumables used were those recommended by ForteBio.

All samples were diluted in freshly prepared running buffer. Antibody variants were immobilized onto the surface of a series of biosensors using the capture methods described. Antigen was passed over the surface to generate a binding response. Binding data for the IgG:antigen interactions were collected at 25° C. on the biosensors. A dilution series of the antigen was used in the association step, in order to fit results globally and get the best values for ka, kd, and KD. The response data for the binding of antigen to the surface immobilized IgG were fitted to a 1:1 binding model. Kinetic parameters are summarized in the table below. HC0 LC0 refers to murine SR-1.

TABLE 7

| | Capture | kₐ | k_{d} | K_{D} | K_{D} | | | Mean |
|---|---|---|---|---|---|---|---|---|
| Antibody | level (nm) | (m⁻¹s⁻¹) | (s⁻¹) | (M) | (nM) | R² | X² | Rₘₐₓ |
| HC0 LC0 | 0.896 | 4.76E+05 | 6.97E−04 | 1.47E−09 | 1.47 | 0.9964 | 0.1449 | 0.1766 |
| HC1 LC1 | 0.854 | 5.03E+05 | 3.97E−04 | 7.90E−10 | 0.79 | 0.9969 | 0.1276 | 0.1774 |
| HC1 LC2 | 0.910 | 5.76E+05 | 6.68E−04 | 1.16E−09 | 1.16 | 0.9953 | 0.2759 | 0.1796 |
| HC1 LC3 | 0.880 | 6.70E+05 | 7.27E−04 | 1.09E−09 | 1.09 | 0.9962 | 0.2069 | 0.1807 |
| HC1 LC5 | 0.877 | 6.28E+05 | 5.52E−03 | 8.79E−09 | 8.79 | 0.9838 | 0.3467 | 0.2420 |
| HC1 LC6 | | | | | | | | |
| HC2 LC1 | 0.905 | 3.99E+05 | 2.06E−03 | 5.16E−09 | 5.16 | 0.9953 | 0.1376 | 0.1959 |
| HC2 LC2 | 0.898 | 4.49E+05 | 4.83E−03 | 1.08E−08 | 10.8 | 0.9959 | 0.1125 | 0.2172 |
| HC2 LC3 | 0.886 | 5.71E+05 | 4.58E−03 | 8.03E−09 | 8.03 | 0.9946 | 0.1597 | 0.2617 |
| HC2 LC5 | 0.898 | 6.32E+05 | 2.14E−02 | 3.38E−08 | 33.8 | 0.9751 | 0.1935 | 0.0752 |
| HC2 LC6 | | | | | | | | |
| HC3 LC1 | 0.916 | 4.95E+05 | 9.71E−04 | 1.96E−09 | 1.96 | 0.9940 | 0.2410 | 0.2114 |
| HC3 LC2 | 0.883 | 5.77E+05 | 1.78E−03 | 3.09E−09 | 3.09 | 0.9936 | 0.1842 | 0.1905 |
| HC3 LC3 | 0.886 | 6.58E+05 | 2.16E−03 | 3.28E−09 | 3.28 | 0.9940 | 0.1679 | 0.1639 |
| HC3 LC5 | 0.881 | 7.15E+05 | 1.87E−02 | 2.62E−08 | 26.2 | 0.9776 | 0.2352 | 0.1176 |
| HC3 LC6 | | | | | | | | |
| HC4 LC1 | | | | | | | | |
| HC4 LC2 | | | | | | | | |
| HC4 LC3 | | | | | | | | |
| HC4 LC5 | | | | | | | | |
| HC4 LC6 | | | | | | | | |
| HC5 LC1 | 0.879 | 5.00E+05 | 9.61E−04 | 1.92E−09 | 1.92 | 0.9855 | 0.3870 | 0.1581 |
| HC5 LC2 | 0.873 | 4.72E+05 | 1.70E−03 | 3.61E−09 | 3.61 | 0.9962 | 0.1484 | 0.2045 |
| HC5 LC3 | 0.844 | 5.89E+05 | 2.16E−03 | 3.67E−09 | 3.67 | 0.9964 | 0.1416 | 0.1517 |
| HC5 LC5 | 0.848 | 6.26E+05 | 1.84E−02 | 2.94E−08 | 29.4 | 0.9734 | 0.1986 | 0.2261 |
| HC5 LC6 | | | | | | | | |

Five of the antibodies showed a dissociation constant KD that was within the range of 2-fold as compared to the original antibody SR-1 (HC0 LC0), namely combinations VH1/VL1, VH1/VL2, VH1/VL3, VH3/VL1 and VH5/VL1. Three of the antibodies even showed a dissociation constant KD that was improved compared to original antibody SR-1, namely combinations VH1/VL1, VH1/VL2 and VH1/VL3.

### Example 2.5: Biophysical Characterization of the Five Preselected Antibodies

It is accepted that protein aggregation, along the non-native pathway, is caused by protein conformational instability, or partial unfolding of a protein. The unfolding of the protein exposes the hydrophobic core residues, which then come together with other partially unfolded monomers to form dimers, and ultimately act as a nucleation point for mass aggregation. Thus, conformational stability is indicative of a protein's propensity for non-native aggregation. Thermal stability is accepted as being a viable method to monitor a protein's conformational stability, through the determination of its melting point (Tm).

Differential scanning fluorimetry (DSF) allows for monitoring of conformational stability. Multiple studies have found highly correlative data between differential scanning calorimetry (DSC) and DSF, suggesting that DSF can successfully monitor protein thermal stability, and ultimately non-native aggregation propensity. DSF uses a fluorescent dye, which is quenched in aqueous environments, but fluoresces under hydrophobic conditions. As such, under heat exposure, antibodies begin to lose conformational stability and unfold, exposing hydrophobic core residues, which can be measured as an increase in fluorescent signal. Temperature vs fluorescence (melting profile) plots can then be used to determine the melting temperature (dF/dT curve) of a protein of interest.

Many antibody molecules exhibit a two-phase melting profile. Published studies have shown mAbs display distinctive unfolding profiles as a result of melting transitions of their individual domains: CH2, CH3, and Fab. The thermal stability of IgG molecules is affected by all these domains. DSF curves reported for antibodies display two clearly discernible transitions: a lower temperature representing unfolding of the CH2 domain (Tm1), and a higher temperature representing melting of CH3-Fab domains (Tm2). Fab melting transition is generally well defined with a peak up to 2-3 times greater than that of CH2 or CH3.

For DSF analysis, solutions of 5 μl of Sypro Orange (diluted 1/200 in water; Sigma) and 45 ul of 0.3 mg/ml of the antibody to be tested were added to a tube (Bio-Rad; TLS0831). The tubes were sealed with optical flat cap (Bio-Rad; TCS0803) and heated in an i-Cycler iQ5 real-time PCR detection system (Bio-Rad) from 20 to 95° C. in increments of 0.5° C. Fluorescence changes in the wells of the plate were monitored simultaneously with a charge-coupled (CCD) camera. The wavelengths for excitation and emission were 485 and 575 nm, respectively. The temperature midpoint for the protein unfolding transition, Tm, was calculated using the Bio-Rad iQ5 software.

For size exclusion chromatography (SEC), samples were diluted to a final concentration of 0.1 mg/ml using phosphate buffered saline (PBS). Highly purified samples of antibodies were loaded independently onto a Superdex 200 increase 10/300 GL gel-filtration column. 50 μl samples were injected and the column flow rate was maintained at 0.75 ml/min. Separations and equilibration steps were performed in phosphate buffered saline at 19° C. Protein peaks were monitored using absorbance at 214 nm and spectra were analyzed using the Unicorn evaluation software package (Cytiva).

Melting curves were successfully generated for all samples. All antibodies tested display single-phase transition melting profiles. Tm values are summarized in the table below.

Also SEC analysis has been performed successfully for all samples. The peak quality for SEC analysis was good all samples. All antibodies eluted as one major peak. From the column calibration, the major peak corresponds to monomeric IgG.

TABLE 8

| Antibody | Tm1 [° C.] | Tm2 [° C.] | % Monomer | Comment |
|---|---|---|---|---|
| VH0/VL0 | 74.0 | N/A | >98 | Good peak |
| | | | | quality |
| VH1/VL1 | 69.3 | N/A | >98 | Good peak |
| | | | | quality |
| VH1/VL2 | 67.0 | N/A | >98 | Good peak |
| | | | | quality |
| VH1/VL3 | 68.3 | N/A | >98 | Good peak |
| | | | | quality |
| VH3/VL1 | 70.3 | N/A | >98 | Good peak |
| | | | | quality |
| VH5/VL1 | 68.7 | N/A | >98 | Good peak |
| | | | | quality |

### Example 2.6: Summary and Selection of the Final Candidate for Further Engineering

Except for the combinations which contain VH4 and/or VL6 all combinations could be successfully expressed and purified. From the SDS-PAGE analysis, all antibodies exhibit adequate levels of purity. Under reducing conditions, both heavy and the light chains of the antibody are visible and are observed at the expected molecular weight of ~50 and 25 kDa, respectively. Under non-reducing conditions, a single major band and several minor bands are observed. The additional bands (impurities) are likely the result of non-glycosylated IgG and IgG degradation products (e.g. a single [partial]light chain, a combination of two heavy and one light chain, two heavy chains, two heavy and one light chain).

In the kinetic (Octet) analysis, combinations VH1/LC1, VH1/LC2, VH1/LC3, VH3/LC1 and VH5/LC1 exhibit binding characteristics similar to that of the control antibody HCO LCO, exhibiting dissociation constants within 2-fold of the control antibody.

The biophysical properties of all antibodies were as good as that as of the original SR-1 antibody.

For further engineering antibody VH1/LC1 was selected which showed an affinity of 0.79 nM, which is almost 2-fold higher than that of the original SR-1 antibody (1.47 nM).

### Example 3: Removal of Individual Sites that are Prone to PTM or Fragmentation

### Example 3.1: Design of the Engineered, Humanized Variants

In the next engineering step, antibodies were screened for sites that are prone to Fv glycosylation, deamidation, isomerization, fragmentation or other kinds of post-translational modification (PTM) sequence hot spots. Several such sites were identified in the CDRs of the antibodies. See the following table.

TABLE 9

| Motif | Type | Occurrence | CDR |
|---|---|---|---|
| NS | deamidation | RASESVDIYGNSFMH | LCDR1 |
| | | (SEQ ID No. 7) | |
| NN | deamidation | QONNEDPYT (SEQ ID No. 9) | LCDR3 |
| DP | Isomerization, | QQNNEDPYT (SEQ ID No. 9) | LCDR3 |
| | fragmentation | | |
| NG | deamidation | VIYSGNGDTSYNQKFKG | HCDR2 |
| | | (SEQ ID No. 5) | |

To remove these detrimental amino acid motifs, variants of antibody VH1/VL1 were generated. The approach for the respective changes were rationalized by analyses as described in the following.

Sequence and structure-based descriptors were computed in silico to identify suitable variants that would preserve the structural integrity and bioactivity of the antibody while enhancing developability, binding affinity, solubility. To this aim, physicochemical descriptors were assessed against a dataset of therapeutic antibodies retrieved from the TABS database (https://tabs.craic.com/) and a second dataset consisting of paired antibody repertoires from the Observed Antibody Space database (http://opig.stats.ox.ac.uk/webapps/oas/). Sequence alignments, antibody numbering and CDR canonical structures were annotated upon alignment of the variable domain sequences to isotype specific Hidden Markov Models (HMMs) built as described in Nat Protoc (2014) 9: 2771-83. Three-dimensional models were built based on methods described in Nucleic Acid Res (2017) 45: W17-W23 and Bioinformatics (2014) 30: 2733-40, using a template-based approach for the frameworks, canonical structure modelling for the CDRs and a random-forest machine learning modelling approach for the heavy chain loop H3. For the VH/VL packing, a template based approach based on global similarity and identity at residue position L44 as described in FEBS J. (2011) 278: 2858-66 was used. Among the sequence descriptors, the antibody solubility profile was computed as a linear combination of different physicochemical properties such as hydrophobicity, electrostatic charge at neutral pH, α-helix and β-strand propensity of each amino acid (Nat Struct Biol (1996) 3: 842-8, J Mol Biol (1994) 238: 693-708). In order to account for the effect of neighboring amino acids, we employed a sliding window approach where the per-residue solubility descriptor is averaged over a window of seven consecutive amino acids and corrected for the occurrence of polar/non-polar aggregation prone patterns J Mol Biol (2000) 296: 961-8; Methods Mol Biol (2022) 2313: 57-113. While during the engineering step we did not impose a specific target value on local and global solubility, we excluded variants that would lead to the formation of covalent aggregates (cysteines) and solvent-exposed hydrophobic patches, as assessed by per-residue relative solvent accessibility computed on the 3D model of the paired VH/VL domain (F1000Res (2016) 5:189). Additionally, variants that would impact the conformations of the CDRs and consequently the antibody binding mode, were also excluded. This was performed by assessing the contribution of the engineered site to any of the established canonical structures (Nat Protocol (2014) 9: 2771-83) as well as by the evaluation of the expected binding mode of the native and engineered molecule using a per-residue probability score (Bioinformatics (2013) 29: 2285-91). Additionally, we excluded/prioritised variants that would lead to increased/reduced immunogenicity risk, as assessed by comparing the immunogenicity profile computed in silico against that of a large dataset of natural human repertoires and therapeutic antibodies of known immunogenicity (ADA frequency). To this aim we employed a readaptation of the NetMHCIIpan neural-network algorithm to screen the native and variant antibody sequences for the presence of class II restricted HLA ligands and putative T cell epitopes (Nucl Acids Res (2020) 48: W449-W454). We computed a global and local immunogenicity profile, corrected by the presence of shared HLA ligands to human germlines, which are likely tolerated or tolerogenic. Higher priority was given to variants that do not carry neo-epitopes (new HLA binders vs native) or cross-reactive epitopes (ligands recognized by multiple HLA alleles), and lower priority to variants that show high epitope content in regions of the antibody that show low immunogenicity scores in the reference antibody datasets.

The following table summarizes the new variants.

TABLE 10

| Antibody | Removal of . . . | Exchange |
|---|---|---|
| VH1/VL1.2 | Deamidation site in LCDR1 | NS => QS |
| VH1/VL1.3 | Deamidation site in LCDR1 | NS => RS |
| VH1/VL1.4 | Deamidation site in LCDR1 | NS => SS |
| VH1/VL1.5 | Deamidation site in LCDR1 | NS => KS |
| VH1/VL1.6 | Deamidation site in LCDR1 | NS => FS |
| VH1/VL1.7 | Deamidation site in LCDR1 | NS => ES |
| VH1/VL1.8 | Deamidation site in LCDR1 | NS => YS |
| VH1/VL1.9 | Deamidation site in LCDR3 | NN => GN |
| VH1/VL1.10 | Deamidation site in LCDR3 | NN => RN |
| VH1/VL1.11 | Deamidation site in LCDR3 | NN => EN |
| VH1/VL1.12 | Deamidation site in LCDR3 | NN => HN |
| VH1/VL1.13 | Deamidation site in LCDR3 | NN => NK |
| VH1/VL1.14 | Isomerization, fragmentation | DP => PP |
| | site in LCDR3 | |
| VH1/VL1.15 | Isomerization, fragmentation | DP => EP |
| | site in LCDR3 | |
| VH1.2/VL1 | Deamidation site in HCDR2 | NG => SG |

Sequences of the new VH and VL chains are shown in the following.

>VL1.2
(SEQ ID NO. 56)
EIVLTQSPSSLSASVGDRVTITCRASESVDIYGQSFMHWYQQKPG
KAPKLLIYLASNLESGVPSRFSGSGSGTDFTLTISSLQPEDFATY
YCQQNNEDPYTFGQGTKLEIKRTV
>VL1.3
(SEQ ID NO. 57)
EIVLTQSPSSLSASVGDRVTITCRASESVDIYGRSFMHWYQQKPG
KAPKLLIYLASNLESGVPSRFSGSGSGTDFTLTISSLQPEDFATY
YCQQNNEDPYTFGQGTKLEIKRTV
>VL1.4
(SEQ ID NO. 58)
EIVLTQSPSSLSASVGDRVTITCRASESVDIYGSSFMHWYQQKPG
KAPKLLIYLASNLESGVPSRFSGSGSGTDFTLTISSLQPEDFATY
YCQQNNEDPYTFGQGTKLEIKRTV
>VL1.5
(SEQ ID NO. 59)
EIVLTQSPSSLSASVGDRVTITCRASESVDIYGKSFMHWYQQKPG
KAPKLLIYLASNLESGVPSRFSGSGSGTDFTLTISSLQPEDFATY
YCQQNNEDPYTFGQGTKLEIKRTV
>VL1.6
(SEQ ID NO. 60)
EIVLTQSPSSLSASVGDRVTITCRASESVDIYGFSFMHWYQQKPG
KAPKLLIYLASNLESGVPSRFSGSGSGTDFTLTISSLQPEDFATY
YCQQNNEDPYTFGQGTKLEIKRTV
>VL1.7
(SEQ ID NO. 61)
EIVLTQSPSSLSASVGDRVTITCRASESVDIYGESFMHWYQQKPG
KAPKLLIYLASNLESGVPSRFSGSGSGTDFTLTISSLQPEDFATY
YCQQNNEDPYTFGQGTKLEIKRTV
>VL1.8
(SEQ ID NO. 62)
EIVLTQSPSSLSASVGDRVTITCRASESVDIYGYSFMHWYQQKPG
KAPKLLIYLASNLESGVPSRFSGSGSGTDFTLTISSLQPEDFATY
YCQQNNEDPYTFGQGTKLEIKRTV
>VL1.9
(SEQ ID NO. 63)
EIVLTQSPSSLSASVGDRVTITCRASESVDIYGNSFMHWYQQKPG
KAPKLLIYLASNLESGVPSRFSGSGSGTDFTLTISSLQPEDFATY
YCQQGNEDPYTFGQGTKLEIKRTV
>VL1.10
(SEQ ID NO. 64)
EIVLTQSPSSLSASVGDRVTITCRASESVDIYGNSFMHWYQQKPG
KAPKLLIYLASNLESGVPSRFSGSGSGTDFTLTISSLQPEDFATY
YCQQRNEDPYTFGQGTKLEIKRTV
>VL1.11
(SEQ ID NO. 65)
EIVLTQSPSSLSASVGDRVTITCRASESVDIYGNSFMHWYQQKPG
KAPKLLIYLASNLESGVPSRFSGSGSGTDFTLTISSLQPEDFATY
YCQQENEDPYTFGQGTKLEIKRTV
>VL1.12
(SEQ ID NO. 66)
EIVLTQSPSSLSASVGDRVTITCRASESVDIYGNSFMHWYQQKPG
KAPKLLIYLASNLESGVPSRFSGSGSGTDFTLTISSLQPEDFATY
YCQQHNEDPYTFGQGTKLEIKRTV
>VL1.13
(SEQ ID NO. 67)
EIVLTQSPSSLSASVGDRVTITCRASESVDIYGNSFMHWYQQKPG
KAPKLLIYLASNLESGVPSRFSGSGSGTDFTLTISSLQPEDFATY
YCQQNKEDPYTFGQGTKLEIKRTV
>VL1.14
(SEQ ID NO. 68)
EIVLTQSPSSLSASVGDRVTITCRASESVDIYGNSFMHWYQQKPG
KAPKLLIYLASNLESGVPSRFSGSGSGTDFTLTISSLQPEDFATY
YCQQNNEPPYTFGQGTKLEIKRTV
>VL1.15
(SEQ ID NO. 69)
EIVLTQSPSSLSASVGDRVTITCRASESVDIYGNSFMHWYQQKPG
KAPKLLIYLASNLESGVPSRFSGSGSGTDFTLTISSLQPEDFATY
YCQQNNEEPYTFGQGTKLEIKRTV
>VH1.2
(SEQ ID NO. 70)
QVQLVQSGAEVKKPGASVKVSCKASGYTFTSYNMHWVRQAPGQGL
EWMGVIYSGSGDTSYNQKFKGRVTMTTDTSTSTAYMELRSLTSDD
TAVYYCARERDTRFGNWGQGTLVTVSA

SEQ ID No.s of the variable light chains including their CDR are shown in the following table.

TABLE 11

| | | LCDR1 | | LCDR2 | | LCDR3 | |
|---|---|---|---|---|---|---|---|
| | VL | Kabat | IMGT | Kabat | IMGT | Kabat | IMGT |
| VL1.2 | 56 | 45 | 48 | 8 | 14 | 9 | 15 |
| VL1.3 | 57 | 73 | 74 | 8 | 14 | 9 | 15 |
| VL1.4 | 58 | 44 | 47 | 8 | 14 | 9 | 15 |
| VL1.5 | 59 | 75 | 76 | 8 | 14 | 9 | 15 |
| VL1.6 | 60 | 77 | 78 | 8 | 14 | 9 | 15 |
| VL1.7 | 61 | 79 | 80 | 8 | 14 | 9 | 15 |
| VL1.8 | 62 | 81 | 82 | 8 | 14 | 9 | 15 |
| VL1.9 | 63 | 7 | 13 | 8 | 14 | 83 | 84 |
| VL1.10 | 64 | 7 | 13 | 8 | 14 | 85 | 86 |
| VL1.11 | 65 | 7 | 13 | 8 | 14 | 87 | 88 |
| VL1.12 | 66 | 7 | 13 | 8 | 14 | 89 | 90 |
| VL1.13 | 67 | 7 | 13 | 8 | 14 | 91 | 92 |
| VL1.14 | 68 | 7 | 13 | 8 | 14 | 93 | 94 |
| VL1.15 | 69 | 7 | 13 | 8 | 14 | 95 | 96 |
| | | HCDR1 | | HCDR2 | | HCDR3 | |
| | VH | Kabat | IMGT | Kabat | IMGT | Kabat | IMGT |
| VH1.2 | 70 | 4 | 10 | 71 | 72 | 6 | 12 |

SEQ ID No. 71:
VIYSGSGDTSYNQKFKG
SEQ ID No. 72:
IYSGSGDT
SEQ ID No. 73:
RASESVDIYGRSFMH
SEQ ID No. 74:
ESVDIYGRSF
SEQ ID No. 75:
RASESVDIYGKSFMH
SEQ ID No. 76:
ESVDIYGKSF
SEQ ID No. 77:
RASESVDIYGFSFMH
SEQ ID No. 78:
ESVDIYGFSF
SEQ ID No. 79:
RASESVDIYGESFMH
SEQ ID No. 80:
ESVDIYGESF
SEQ ID No. 81:
RASESVDIYGYSFMH
SEQ ID No. 82:
ESVDIYGYSF
SEQ ID No. 83:
QQGNEDPYT
SEQ ID No. 84:
QQGNEDPYTF
SEQ ID No. 85:
QQRNEDPYT
SEQ ID No. 86:
QQRNEDPYTF
SEQ ID No. 87:
QQENEDPYT
SEQ ID No. 88:
QQENEDPYTF
SEQ ID No. 89:
QQHNEDPYT
SEQ ID No. 90:
QQHNEDPYTF
SEQ ID No. 91:
QQNKEDPYT
SEQ ID No. 92:
QQNKEDPYTF
SEQ ID No. 93:
QQNNEPPYT
SEQ ID No. 94:
QQNNEPPYTF
SEQ ID No. 95:
QQNNEEPYT
SEQ ID No. 96:
QQNNEEPYTF

### Example 3.2: Testing of the Engineered, Humanized Variants

The engineered variants were subjected to the same type of analysis and characterization as the initially humanized variants. For the experimental set up see Example 2.

All variants could be expressed and purified, but the yield was not sufficiently high for all variants to perform all assays, in particular differential scanning fluorimetry (DSF). Four antibodies showed an increase of the dissociation constant KD of more than 2-fold as compared to the original SR-1 antibody (V71/VL5.3, VH1/VL155, VH1/LC1.10 and VH1/VL1.11). Five of the antibodies showed a dissociation constant KD that was even below that of the original antibody SR-1, namely VH1/LC1.2, VH1/LC1.4, VH/LC17, VH1/LC1 and VH1/LC1.15). Three variants had a monomer content of below 85% (HC1/LC1.2, HC6/LC1.10 and HC9/LC1.21). In DF, some antibodies showed a slightly lower TM1. For some of the antibodies also a TM2 could be measured. Results are summarized in the following table.

TABLE 12

| Antibody | K_{D} [nM] | Tm1 [° C.] | Tm2 [° C.] | % Monomer |
|---|---|---|---|---|
| SR-1 | 0.9 | 69.0 | 74.5 | 90.2 |
| VH1/VL1 | 0.4 | 69.3 | N/A | 98.0 |
| VH1/VL1.2 | 0.3 | N/D | N/D | 81 |
| VH1/VL1.3 | 3.5 | 70.5 | 75.0 | 91.3 |
| VH1/VL1.4 | 0.5 | 70.5 | N/A | 90.1 |
| VH1/VL1.5 | 3.7 | 69.5 | 76.0 | 90.2 |
| VH1/VL1.6 | 1.2 | N/D | N/D | 95 |
| VH1/VL1.7 | 0.2 | 69.5 | N/A | 88 |
| VH1/VL1.8 | 0.2 | N/D | N/D | 96.7 |
| VH1/VL1.9 | 1.3 | 68.5 | 81.5 | 87.8 |
| VH1/VL1.10 | 4 | 68.0 | 83.0 | 81.2 |
| VH1/VL1.11 | 2.2 | 67.0 | 81.5 | 81.15 |
| VH1/VL1.12 | 1.3 | 69.5 | NA | 87.5 |
| VH1/VL1.13 | 1.4 | 69.0 | 81.5 | 90.3 |
| VH1/VL1.14 | 1.4 | N/D | N/D | 93.2 |
| VH1/VL1.15 | 0.4 | 68.5 | 80.0 | 86.7 |
| VH1.2/VL1 | 1.3 | 69.0 | N/A | 99 |

### Example 3.3: Summary and Selection of the Candidates for Further Engineering

In summary, it was possible to remove unwanted motifs from the sequence of humanized variants of SR-1. This was in particular surprising since the sequence motif were located within the CDRs regions. The new variants nevertheless retained their ability to bind the target antigen with about the same, and some variants even with increase affinity, with antibodies VH1/VL1.7 and VH1/VL1.8 showing the highest affinities. At the same time the biophysical properties of the binders remained in an acceptable range.

For further engineering, five antibodies were selected (VH1/VL1.7, VH1/VL1.8, VH1/VL1.13, VH1/VL1.15 and VH1.2/VL1), which overall showed the most promising properties for further derivatization.

### Example 4: Removal of Multiple Sites that are Prone to PTM or Fragmentation

### Example 4.1: Design of the Further Engineered, Humanized Variants

Encouraged by the successful removal of detrimental sequences motifs from the CDRs of humanized variants of SR-1, the inventors next investigated if it would even be possible to remove multiple such motifs without losing affinity or functionality of the binders.

In addition to the site that were engineered in Example 3, also a YL motif in the in the variable light chain was engineered. The following variants were generated.

TABLE 13
(“—” indicates that the site was not changed):

| | NS in | YL in | NN in | DP in | NG in |
|---|---|---|---|---|---|
| Antibody | LCDR1 | VL | LCDR3 | LCDR3 | HCDR2 |
| VH1/VL1.16 | YS | — | — | EP | — |
| VH1/VL1.17 | ES | — | — | EP | — |
| VH1/VL1.18 | YS | — | NK | EP | — |
| VH1/VL1.19 | ES | — | NK | EP | — |
| VH1.2/VL1.16 | YS | — | — | EP | SG |
| VH1.2/VL1.17 | ES | — | — | EP | SG |
| VH1/VL1.25 | QS | — | NK | EP | — |
| VH1/VL1.26 | SS | — | NK | EP | — |
| VH1/VL1.27 | HS | — | NK | EP | — |
| VH1/VL1.28 | SS | YK | NK | EP | — |
| VH1/VL1.29 | ES | — | NR | EP | — |
| VH1.4/VL1.29 | ES | — | NR | EP | QG |

Variable light chains VL1.28 and VL1.29 each contain an additional L-to-K and N-to-R mutation. respectively.

Sequences of the new VH and VL chains are shown in the following.

>VL1.16
(SEQ ID NO. 97)
EIVLTQSPSSLSASVGDRVTITCRASESVDIYGYSFMHWYQQKPG
KAPKLLIYLASNLESGVPSRFSGSGSGTDFTLTISSLQPEDFATY
YCQQNNEEPYTFGQGTKLEIKRTV
>VL1.17
(SEQ ID NO. 98)
EIVLTQSPSSLSASVGDRVTITCRASESVDIYGESFMHWYQQKPG
KAPKLLIYLASNLESGVPSRFSGSGSGTDFTLTISSLQPEDFATY
YCQQNNEEPYTFGQGTKLEIKRTV
>VL1.18
(SEQ ID NO. 99)
EIVLTQSPSSLSASVGDRVTITCRASESVDIYGYSFMHWYQQKPG
KAPKLLIYLASNLESGVPSRFSGSGSGTDFTLTISSLQPEDFATY
YCQQNKEEPYTFGQGTKLEIKRTV
>VL1.19
(SEQ ID NO. 100)
EIVLTQSPSSLSASVGDRVTITCRASESVDIYGESFMHWYQQKPG
KAPKLLIYLASNLESGVPSRFSGSGSGTDFTLTISSLQPEDFATY
YCQQNKEEPYTFGQGTKLEIKRTV
>VL1.25
(SEQ ID NO. 101)
EIVLTQSPSSLSASVGDRVTITCRASESVDIYGQSFMHWYQQKPG
KAPKLLIYLASNLESGVPSRFSGSGSGTDFTLTISSLQPEDFATY
YCQQNKEEPYTFGQGTKLEIKRTV
>VL1.26
(SEQ ID NO. 102)
EIVLTQSPSSLSASVGDRVTITCRASESVDIYGSSFMHWYQQKPG
KAPKLLIYLASNLESGVPSRFSGSGSGTDFTLTISSLQPEDFATY
YCQQNKEEPYTFGQGTKLEIKRTV
>VL1.27
(SEQ ID NO. 103)
EIVLTQSPSSLSASVGDRVTITCRASESVDIYGHSFMHWYQQKPG
KAPKLLIYLASNLESGVPSRFSGSGSGTDFTLTISSLQPEDFATY
YCQQNKEEPYTFGQGTKLEIKRTV
>VL1.28
(SEQ ID NO. 104)
EIVLTQSPSSLSASVGDRVTITCRASESVDIYGSSFMHWYQQKPG
KAPKLLIYKASNLESGVPSRFSGSGSGTDFTLTISSLQPEDFATY
YCQQNKEEPYTFGQGTKLEIKRTV
>VL1.29
(SEQ ID NO. 105)
EIVLTQSPSSLSASVGDRVTITCRASESVDIYGESFMHWYQQKPG
KAPKLLIYLASNLESGVPSRFSGSGSGTDFTLTISSLQPEDFATY
YCQQNREEPYTFGQGTKLEIKRTV
>VH1.4
(SEQ ID NO. 106)
QVQLVQSGAEVKKPGASVKVSCKASGYTFTSYNMHWVRQAPGQGL
EWMGVIYSGQGDTSYNQKFKGRVTMTTDTSTSTAYMELRSLTSDD
TAVYYCARERDTRFGNWGQGTLVTVSA

SEQ ID No.s of the variable light chains including their CDR are shown in the following table.

TABLE 14

| | | LCDR1 | | LCDR2 | | LCDR3 | |
|---|---|---|---|---|---|---|---|
| | VL | Kabat | IMGT | Kabat | IMGT | Kabat | IMGT |
| VL1.16 | 97 | 81 | 82 | 8 | 14 | 95 | 96 |
| VL1.17 | 98 | 79 | 80 | 8 | 14 | 95 | 96 |
| VL1.18 | 99 | 81 | 82 | 8 | 14 | 113 | 114 |
| VL1.19 | 100 | 79 | 80 | 8 | 14 | 113 | 114 |
| VL1.25 | 101 | 45 | 48 | 8 | 14 | 113 | 114 |
| VL1.26 | 102 | 44 | 47 | 8 | 14 | 113 | 114 |
| VL1.27 | 103 | 109 | 110 | 8 | 14 | 113 | 114 |
| VL1.28 | 104 | 44 | 47 | 111 | 112 | 113 | 114 |
| VL1.29 | 105 | 79 | 80 | 8 | 14 | 115 | 116 |
| | | HCDR1 | | HCDR2 | | HCDR3 | |
| | VH | Kabat | IMGT | Kabat | IMGT | Kabat | IMGT |
| VH1.4 | 106 | 4 | 10 | 107 | 108 | 6 | 12 |

SEQ ID No. 107:
VIYSGQGDTSYNQKFKG
SEQ ID No. 108:
IYSGQGDT
SEQ ID No. 109:
RASESVDIYGHSFMH
SEQ ID No. 110:
ESVDIYGHSF
SEQ ID No. 111:
KASNLES
SEQ ID No. 112:
KAS
SEQ ID No. 113:
QQNKEEPYT
SEQ ID No. 114:
QQNKEEPYTF
SEQ ID No. 115:
QQNREEPYT
SEQ ID No. 116:
QQNREEPYTF

### Example 4.2: Testing of the Further Engineered, Humanized Variants

The engineered variants were subjected to the same type of analysis and characterization as the initially humanized variants. For the experimental set up see Example 2.

All variants could be expressed and purified in with sufficient yield. Two antibodies showed an increase of the dissociation constant K_{D} of more than 2-fold as compared to the original SR-1 antibody (VH1/VL1.27 and VH1/VL1.28). Five of the new antibodies showed a dissociation constant Ka that was at least 2-fold below that of the original antibody SR-1, namely VH1/VL1.16, VH1/VL1.17, VH1/VL1.19, VH1.2/VL1.16 and VH1.2/VL1.17). The monomer content of all antibodies was above 92%. In SEC, for all antibodies a TM2 could be measured. Results are summarized in the following table.

TABLE 15

| Antibody | K_{D} [nM] | Tm2 [° C.] | % Monomer |
|---|---|---|---|
| SR-1 | 0.91 | 75 | 96.3 |
| VH1/VL1.16 | 0.13 | 80.5 | 96.5 |
| VH1/VL1.17 | 0.19 | 80.0 | 93.8 |
| VH1/VL1.18 | 0.56 | 83.5 | 95.5 |
| VH1/VL1.19 | 0.45 | 83.5 | 92.7 |
| VH1.2/VL1.16 | 0.37 | 81.0 | 95.3 |
| VH1.2/VL1.17 | 0.44 | 80.0 | 93.5 |
| VH1/VL1.25 | 1.1 | 84.0 | 92.3 |
| VH1/VL1.26 | 1.6 | 84.0 | 97.7 |
| VH1/VL1.27 | 3.3 | 84.0 | 89.3 |
| VH1/VL1.28 | 8.3 | 84.0 | 95.7 |
| VH1/VL1.29 | 1.1 | 82.8 | 98.9 |
| VH1.4/VL1.29 | 1.3 | 82.5 | 100 |

### Example 4.3: Summary and Selection of the Candidates for Further Engineering

in summary, it was even possible to remove several unwanted motifs from the sequence of humanized variants of SR-1. At the same time the affinity of many variants was even higher than that of the original SR-1 antibody, and the biophysical properties of the binders remained in the range of that of SR.1-

Several antibodies appear to be particular preferred (VH1/VL1.17, VH1/VL1.19, VH1/VL1.27 and VH1.4/VL129) which show the most promising properties. In each of these antibodies at least two detrimental sequence motifs were removed: the deamidation site in the LCDR1 (NS to ES in VH1/VL1.17, VH1/VL119 and VH1.4/VL1.29, and NS to HS in VH1/VL1.27), and the isomerization/fragmentation site in LCDR3 (DP to EP in all mentioned binders). Additionally, the deamidation site in the LCDR3 was changed from NN to NK in VH1/VL1.19 and VH1/VL1.27 and from NN to NR in VH1.4/VL1.29. Also, in binder VH1.4/VL1.29 the deamidation site in HCDR2 was removed (NG to QG).

Also particular preferred are antibodies that have an affinity as high, or even higher, than antibody SR-1, namely VH1/VL1.17, VH1/VH1.18, VH1/VL1.19, VH1.2/VL1.17, and VH1/VL1.16.

### Example 5: Additional Sequence Variations

### Example 5.1: Design of the Further Engineered, Humanized Variants

Based on the results and the experience obtained so far, further sequence variations were designed. These mutations aim to further combine additional mutations, and also to increase hydrophilicity.

The following variants were generated.

TABLE 16
(“—” indicates that the site was not changed)

| | NS in | YL in | NN in | DP in | NG in |
|---|---|---|---|---|---|
| Antibody | LCDR1 | VL | LCDR3 | LCDR3 | HCDR2 |
| VH1.4/VL1.30 | ES | YH | NR | EP | QG |
| VH1.4/VL1.31 | ES | YE | NR | EP | QG |
| VH1.4/VL1.32 | ES | YS | NR | EP | QG |
| VH1.4/VL1.33 | ES | YQ | NR | EP | QG |
| VH1.4/VL1.19 | ES | — | NK | EP | QG |

Additionally, binders VH1.4/VL1.30, VH1.4/VL1.31, VH1.4/VL1.32 and VH1.4/VL1.33 contain a L-to-H, a L-to-H, a L-to-H, and a L-to-H mutation, respectively, to increase hydrophilicity.

Sequences of the new VH and VL chains are shown in the following.

>VL1.30
(SEQ ID NO. 117)
EIVLTQSPSSLSASVGDRVTITCRASESVDIYGESFMHWYQQKPG
KAPKLLIYHASNLESGVPSRFSGSGSGTDFTLTISSLQPEDFATY
YCQQNREEPYTFGQGTKLEIK
>VL1.31
(SEQ ID NO. 118)
EIVLTQSPSSLSASVGDRVTITCRASESVDIYGESFMHWYQQKPG
KAPKLLIYEASNLESGVPSRFSGSGSGTDFTLTISSLQPEDFATY
YCQQNREEPYTFGQGTKLEIK
>VL1.32
(SEQ ID NO. 119)
EIVLTQSPSSLSASVGDRVTITCRASESVDIYGESFMHWYQQKPG
KAPKLLIYSASNLESGVPSRFSGSGSGTDFTLTISSLOPEDFATY
YCQQNREEPYTFGQGTKLEIK
>VL1.33
(SEQ ID NO. 120)
EIVLTQSPSSLSASVGDRVTITCRASESVDIYGESFMHWYQQKPG
KAPKLLIYQASNLESGVPSRFSGSGSGTDFTLTISSLQPEDFATY
YCQQNREEPYTFGQGTKLEIK

SEQ ID No.s of the variable light chains including their CDR are shown in the following table.

TABLE 17

| | | LCDR1 | | LCDR2 | | LCDR3 | |
|---|---|---|---|---|---|---|---|
| | VL | Kabat | IMGT | Kabat | IMGT | Kabat | IMGT |
| VL1.30 | 117 | 79 | 80 | 121 | 122 | 115 | 116 |
| VL1.31 | 118 | 79 | 80 | 123 | 124 | 115 | 116 |
| VL1.32 | 119 | 79 | 80 | 125 | 126 | 115 | 116 |
| VL1.33 | 120 | 79 | 80 | 127 | 128 | 115 | 116 |

SEQ ID No. 121:
HASNLES
SEQ ID No. 122:
HAS
SEQ ID No. 123:
EASNLES
SEQ ID No. 124:
EAS
SEQ ID No. 125:
SASNLES
SEQ ID No. 126:
SAS
SEQ ID No. 127:
QASNLES
SEQ ID No. 128:
QAS

### Example 5.2: Testing of the Further Engineered, Humanized Variants

The engineered variant were subjected to the same type of analysis and characterization as the initially humanized variants. For the experimental set up see Example 2.

All variants can be expressed and purified in with sufficient yield. The antibodies show an affinity as measured by the dissociation constant K_{D} in the range of the original SR-1 antibody. Numerous antibodies even have an increased affinity. The biophysical properties of the antibodies are good.

### Example 5.3: Summary and Selection of the Candidates for Further Engineering

In summary, it was even possible to remove several unwanted motifs from the sequence of humanized variants of SR-1. At the same time the affinity of many variants was even higher than that of the original SR-1 antibody. At the same time the biophysical properties of the binders remained in the range of that of SR-1.

Certain antibodies appear to be particular preferred (VH1/VL1.17, VH1/VL1.19, VH1/VL1.27 and VH1/LC1.29) which show the most promising properties. In each of these antibodies detrimental sequence motifs were removed: the deamidation site in the LCDR1 (NS to ES VH1/VL1.17, VH1/VL1.19 and VH1.4/VL1.29, and NS to HS in VH1/VL1.27), the deamidation site in the LCDR3 (NN to NK in VH1/VL1.19 and VH1/VL1.27, and NN to NR in VH1.4/VL1.29), and the isomerization/fragmentation site in LCDR3 (DP to EP in all aformentioned binders). Additionally, in binder VH1.4/VL1.29 the deamidation site in HCDR2 was removed (NG to G).

### Example 6: Summary of the Sequences of the Antibodies Disclosed in the Present Patent

The following table summarizes the key antibodies produced and tested in the present disclosure. Indicated are the VH and the VL sequences of the antibodies. The respective VH sequences, the VL sequences, and the CDR sequences can be found in the preceding Examples.

TABLE 18

| | | VH SEQ | | VL SEQ |
|---|---|---|---|---|
| Antibody | VH | ID No. | VL | ID No. |
| Dufour | VH1 | 22 | VL1 | 38 |
| Zumstein | VH1 | 22 | VL2 | 39 |
| Signalkuppe | VH1 | 22 | VL3 | 40 |
| Dom | VH3 | 24 | VL1 | 38 |
| Liskamm | VH5 | 26 | VL1 | 38 |
| Weisshorn | VH1 | 22 | VL1.2 | 56 |
| Täschhorn | VH1 | 22 | VL1.3 | 57 |
| Matterhorn | VH1 | 22 | VL1.4 | 58 |
| Parrot | VH1 | 22 | VL1.5 | 59 |
| Dent Blanche | VH1 | 22 | VL1.6 | 60 |
| Ludwigshöhe | VH1 | 22 | VL1.7 | 61 |
| Nadelhorn | VH1 | 22 | VL1.8 | 62 |
| Grand Combin | VH1 | 22 | VL1.9 | 63 |
| Finsteraarhor | VH1 | 22 | VL1.10 | 64 |
| Castor | VH1 | 22 | VL1.11 | 65 |
| Zinalrithorn | VH1 | 22 | VL1.12 | 66 |
| Alphubel | VH1 | 22 | VL1.13 | 67 |
| Aletschhorn | VH1 | 22 | VL1.14 | 68 |
| Breithorn | VH1 | 22 | VL1.15 | 69 |
| Jungfrau | VH1.2 | 70 | VL1 | 38 |
| Mönch | VH1.3 | 24 | VL1 | 38 |
| Schreckhorn | VH1 | 22 | VL1.16 | 97 |
| Piz Bernina | VH1 | 22 | VL1.17 | 98 |
| Dirruhorn | VH1 | 22 | VL1.18 | 99 |
| Allalinhorn | VH1 | 22 | VL1.19 | 100 |
| Weissmies | VH1.2 | 70 | VL1.16 | 97 |
| Lagginhorn | VH1.2 | 70 | VL1.17 | 98 |
| Piz Zupo | VH1 | 22 | VL1.25 | 101 |
| Eiger | VH1 | 22 | VL1.26 | 102 |
| Piz Argient | VH1 | 22 | VL1.27 | 103 |
| Piz Roseg | VH1 | 22 | VL1.28 | 104 |
| Bietschhorn | VH1 | 22 | VL1.29 | 105 |
| Bellavista | VH1.4 | 106 | VL1.29 | 105 |
| Piz Palü | VH1.4 | 106 | VL1.30 | 117 |
| Nesthorn | VH1.4 | 106 | VL1.31 | 118 |
| Dreieckhorn | VH1.4 | 106 | VL1.32 | 119 |
| Piz Morteratsch | VH1.4 | 106 | VL1.33 | 120 |
| Balmhorn | VH1.4 | 106 | VL1.19 | 100 |

### Example 7: Antibody-Mediated Inhibition of SCF-Dependent Proliferation

SCF-dependent proliferation is a function of CD117. The humanized antibodies of the present disclosure were tested for their ability to inhibit SCF-dependent proliferation in comparison to the original SR-1 antibody and some other prior art antibodies. In addition to the SR-1 antibody, which is described above, the antibodies in Table 19 were tested in comparison to the antibodies of the present disclosure.

TABLE 19

| | | |
|---|---|---|
| SEQ ID | | |
| No. | Comment | Sequence |

NegCtrl

| | | |
|---|---|---|
| 129 | VH | QVQLQQSGPGLVKPSQTLSLTCAISGDSVSSNSAAWSWIRQSPGRG |
| | | LEWLGRIYYRSKWYNDYAVSVKSRITINPDTSKNQFSLQLNSVTPEDT |
| | | AVYYCARLDHRYHEDTVYPGMDVWGQGTLVTVSS |
| 130 | VL | DIELTQPPSVSVAPGQTARISCSGDNLPAYTVTWYQQKPGQAPVLVI |
| | | YDDSDRPSGIPERFSGSNSGNTATLTISGTQAEDEADYYCASWDPSS |
| | | GVVFGGGTKLTVL |
| 131 | HCDR1 (Kabat) | SNSAAWS |
| 132 | HCDR2 (Kabat) | RIYYRSKWYNDYAVSVKS |
| 133 | HCDR3 (Kabat) | LDHRYHEDTVYPGMDV |
| 134 | LCDR1 (Kabat) | SGDNLPAYTVT |
| 135 | LCDR2(Kabat) | DDSDRPS |
| 136 | LCDR3 (Kabat) | ASWDPSSGVV |
| 137 | HCDR1 (IMGT) | GDSVSSNSAA |
| 138 | HCDR2 (IMGT) | IYYRSKWYN |
| 139 | HCDR3 (IMGT) | ARLDHRYHEDTVYPGMDV |
| 140 | LCDR1 (IMGT) | NLPAYT |
| 141 | LCDR2 (IMGT) | DDS |
| 136 | LCDR3 (IMGT) | ASWDPSSGVV |

AMG191

| | | |
|---|---|---|
| 142 | VH | QVQLVQSGAEVKKPGASVKVSCKASGYTFTSYNMHWVRQAPGQG |
| | | LEWMGVIYSGNGDTSYNQKFKGRVTITADKSTSTAYMELSSLRS |
| | | EDTAVYYCARERDTRFGNWGQGTLVTVSS |
| 143 | VL | DIVMTQSPDSLAVSLGERATINCRASESVDIYGNSFMHWYQQKPGQ |
| | | PPKLLIYLASNLESGVPDRFSGSGSGTDFTLTISSLQAEDVAVYYC |
| | | QQNNEDPYTFGGGTKVEIKR |
| 4 | HCDR1 (Kabat) | SYNMH |
| 5 | HCDR2 (Kabat) | VIYSGNGDTSYNQKFKG |
| 6 | HCDR3 (Kabat) | ERDTRFGN |
| 7 | LCDR1 (Kabat) | RASESVDIYGNSFMH |
| 8 | LCDR2(Kabat) | LASNLES |
| 9 | LCDR3 (Kabat) | QQNNEDPYT |
| 10 | HCDR1 (IMGT) | GYTFTSYN |
| 11 | HCDR2 (IMGT) | IYSGNGDT |
| 12 | HCDR3 (IMGT) | ARERDTREGN |
| 13 | LCDR1 (IMGT) | ESVDIYGNSF |
| 14 | LCDR2 (IMGT) | LAS |
| 9 | LCDR3 (IMGT) | QQNNEDPYT |

FSI-174

| | | |
|---|---|---|
| 144 | VH | QVQLVQSGAEVKKPGASVKVSCKASGYTFTSYNMHWVRQAPGQG |
| | | LEWMGVIYSGNGDTSYAQKFQGRVTITADKSTSTAYMELSSLRS |
| | | EDTAVYYCARERDTRFGNWGQGTLVTVSS |
| 145 | VL | DIVMTQSPLSLPVTPGEPASISCRASESVDIYGQSFMHWYLQKP |
| | | GQSPQLLIYLASNLESGVPDRFSGSGSGTDFTLKISRVEAEDVG |
| | | VYYCQQNNEDPYTFGGGTKVEIK |
| 4 | HCDR1 (Kabat) | SYNMH |
| 146 | HCDR2 (Kabat) | VIYSGNGDTSYAQKFQG |
| 6 | HCDR3 (Kabat) | ERDTRFGN |
| 45 | LCDR1 (Kabat) | RASESVDIYGQSFMH |
| 8 | LCDR2(Kabat) | LASNLES |
| 9 | LCDR3 (Kabat) | QQNNEDPYT |
| 10 | HCDR1 (IMGT) | GYTFTSYN |
| 11 | HCDR2 (IMGT) | IYSGNGDT |
| 12 | HCDR3 (IMGT) | ARERDTREGN |
| 48 | LCDR1 (IMGT) | ESVDIYGQSF |
| 14 | LCDR2 (IMGT) | LAS |
| 15 | LCDR3 (IMGT) | QQNNEDPYT |

NegCtrl is a human IgG1 control antibody specific for chicken lysozyme. AMG191 and FSI-174 are human IgG1 antibodies specific for human CD117. AMG191 is a humanized derivative of SR-1, but the six CDRs are identical to SR-1, i.e. AMG191 still contains the critical sites in the CDRs that have been removed in the humanized antibodies of the present disclosure. FSI-174 is also a humanized derivative of SR-1. Compared to SR-1, FSI-174 has two amino acid differences in the HCDR2 and one amino acid difference in the LCDR1. These changes were introduced to bring them closer to the human germline sequences, thereby increasing the humanness of the antibodies. One of the changes introduced into FSI-174 coincidently removes a deamidation site in the LCDR1. The LCDR3 of FSI-174 is however identical to that of SR-1, i.e. the deamidation hotspot and the isomerization/fragmentation hotspot in the LCDR3 are still present in FSI-174. The CDRs (according to Kabat) of SR-1, AMG191 and FSI-174 are shown in Table 20.

TABLE 20

| Anti- | | | | | | |
|---|---|---|---|---|---|---|
| body | HCDR | HCDR2 | HCDR3 | LCDR1 | LCDR2 | LCDR3 |
| SR-1 | SYNMH | VIYSGNG | ERDT | RASESVDI | LASN | QQNN |
| | (SEQ | DTSYNQK | RFGN | YGNSFMH | LES | EDPYT |
| | ID | FKG | (SEQ | (SEQ | (SEQ | (SEQ |
| | No. | (SEQ | ID | ID | ID | ID |
| | 4) | ID | No. | No. | No. | No. |
| | | No. | 6) | 7) | 8) | 9) |
| | | 5) | | | | |
| AMG191 | SYNMH | VIYSGNG | ERDT | RASESVDI | LASN | QQN |
| | (SEQ | DTSYNQK | RFGN | YGNSFMH | LES | NED |
| | ID | FKG | (SEQ | (SEQ | (SEQ | PYT |
| | No. | (SEQ | ID | ID | ID | (SEQ |
| | 4) | ID | No. | No. | No. | ID |
| | | No. | 6) | 7) | 8) | No. |
| | | 5). | | | | 9) |
| FSI- | SYNMH | VIYSGNG | ERDT | RASESV | LASN | QQNN |
| 174 | (SEQ | DTSYAQK | RFGN | DIYGQS | LES | EDP |
| | ID | FQG | (SEQ | FMH | (SEQ | YT |
| | No. | (SEQ | ID | (SEQ | ID | (SEQ |
| | 4) | ID | No. | ID | No. | ID |
| | | No. | 6) | No. | 8) | No. |
| | | 146) | | 45) | | 9) |

SCF-dependent proliferation is measured as follows: TF-1 cells were seeded at a cell concentration of 150,000 cells/ml in sterile, white cell culture treated plates in RPMI Media+10% FCS. Cells were treated with 100 ng/ml of SCF and a titration of various CD117 antibodies or an isotype control antibody at different concentrations. After 3 days of incubation a CellTiter-Glo 2.0 assay (Promega) was performed and luminescence is read in a luminometer (Envision, Perkin Elmer), and IC50's were determined. Results are shown in Table 21.

TABLE 21

IC50 [μg/ml]

| Antibody | Experiment #1 | Experiment #2 | Average |
|---|---|---|---|
| NegCtrl | >0.5 | >0.5 | >0.5 |
| SR-1 | 0.0924 | 0.4597 | 0.2760 |
| AMG191 | 0.0930 | 0.3232 | 0.2081 |
| FSI-174 | 0.0387 | 0.0560 | 0.0473 |
| Dufour | 0.1196 | 0.3206 | 0.2201 |
| Matterhorn | 0.0648 | 0.1680 | 0.1164 |
| Piz Bernina | 0.0122 | 0.0231 | 0.0176 |
| Allalinhorn | 0.0415 | 0.0467 | 0.0441 |
| Piz Zupo | >0.5 | >0.5 | >0.5 |
| Eiger | >0.5 | >0.5 | >0.5 |
| Piz Argient | >0.5 | >0.5 | >0.5 |
| Piz Roseg | >0.5 | >0.5 | >0.5 |
| Bietschhorn | 0.0455 | 0.2562 | 0.1509 |
| Bellavista | 0.1026 | 0.2119 | 0.1573 |

Several of the antibodies generated showed an IC50 in this SCF-dependent proliferation assay that is in the range of, or even below that of the original SR-1 antibody, as well as the known humanized derivatives thereof.

### Example 8: Antibody-Mediated Inhibition of SCF-Dependent Phosphorylation

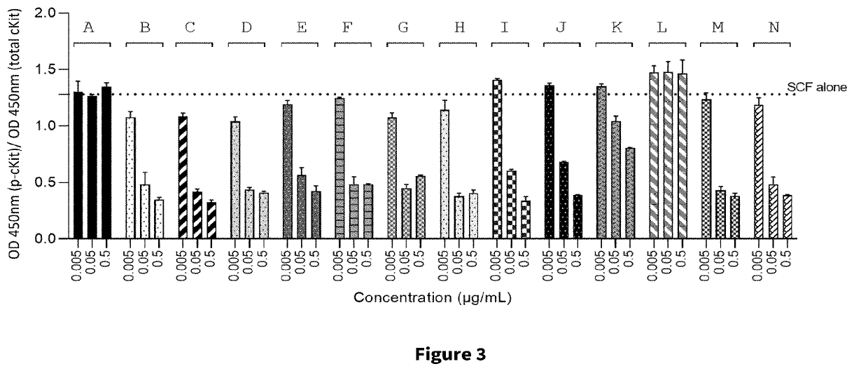

SCF-dependent phosphorylation is another function of CD117. Antibody-mediated inhibition of SCF-dependent phosphorylation is measured using TF-1 cells expressing CD117. TF-1 cells were seeded at a concentration of 1 million cells/mL in 2 mL of Medium (RPM11640 supplemented with GlutaMAX+10% heat inactivated FBS) in a 6-well plate. Cells were pre-treated with 0.005, 0.05 and 0.5 ug/mL antibody for 30 min, before adding of 100 ng/mL recombinant human SCF (1 mg/mL PeproGMP© Recombinant Human SCF) for 5 minutes. Cells were collected, washed with ice-cold PBS, before resuspending cells in 500 μL Lysis Buffer (Lysis buffer from Signaling containing Protease Inhibitor and Phosphatase Inhibitor). Cells were flash frozen in liquid nitrogen and thawed at 37° C. twice, before freezing them at −80° C. until further usage. Undiluted samples were thawed and processed according to the manufacturers protocol for the PathScan® Phospho-c-Kit (Tyr719) Sandwich ELISA Kit (Cell Signaling) and Human c-Kit (CD117) ELISA Kit (Abcam). Absorbance (OD_450 nm) was assessed using the Envision (Perkin Elmer). Tested were the same antibodies as in Example 7. Results are shown in FIG. 3.

As can be seen, most antibodies of the present disclosure tested inhibited SCF-dependent phosphorylation at least as strongly as the SR-1 antibody and the humanized derivatives AMG191 and FSI-174.

### Example 9: Inhibition of SCF-Binding in TF-1 Cells

In this experiment it was tested if the antibodies of the present disclosure are able to inhibit binding of SCF to CD117.

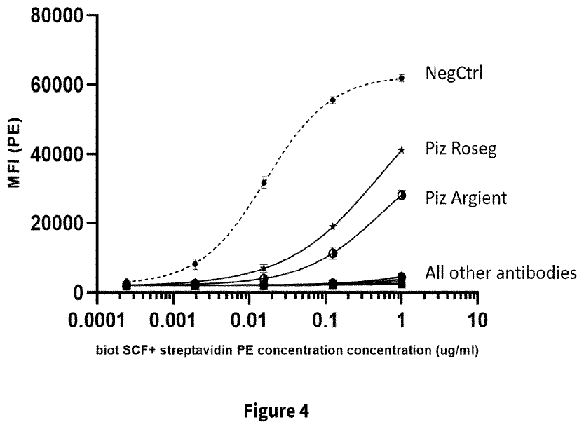

To do so, TF-1 wild-type cells were seeded at a cell concentration of 100,000 cells/well. A Fc receptor blocking was performed using 2 5 μL TruStain FcX (BioLegend). Next, the cells were treated with a fixed concentration of 50 μg/mi of the antibodies to be tested. After 15 min a mix of varying concentrations of biotinylated SCF (Acro-Biosystems) and streptavidin PE (Invitrogen) was added. After additional 30 min cells were washed, and a live dead staining via 7AAD (7-aminoactinomycin D) (BioLegend) was performed using 0.5 μg/mL 7AAD for 10 min before analyzing cells in a flow cytometer. Tested were the following antibodies: NegCtrl, SR-1, AMG191, FSI-174, Dufour, Matterhorn, Piz Bernina, Allalinhorn, Piz Zupo, Eiger, Piz Argient, Piz Roseg, Bietschhorn and Bellavista. Results are shown in FIG. 4.

Except Piz Roseg and Piz Argient, all antibodies of the present disclosure effectively inhibited binding of SCF to CD117. The NegCtrl antibody (anti-chicken lysozyme) did not inhibit binding of SCF to CD117.

### Example 10: Binding of the Antibodies to CD117 in TF-1 Cells

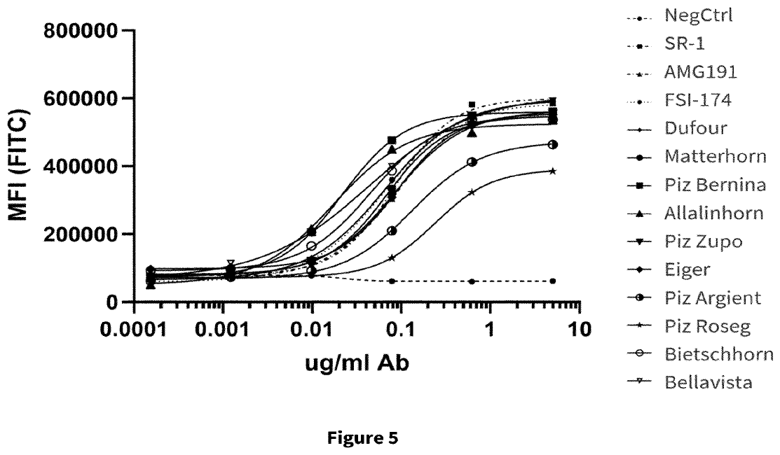

Binding of the antibodies of the present disclosure to CD117 on TF-1 cells was measured. TF-1 wild-type cells were seeded at a concentration of 100,000 cells/well. A Fc receptor blocking was performed using 25 μL TruStain FcX (BioLegend) for 15 min. Next cells are treated with various concentrations of the antibodies to be tested (0.0001-5 μg/ml). After additional 30 min cells were washed, then a secondary antibody was added to the cells (IgG (H+L) Cross-Adsorbed Goat anti-Human, Alexa Fluor® 488). After 30 min and two additional wash steps a live dead staining via 7AAD (7-aminoactinomycin D) was performed using 0.5 μg/mL 7AAD for 10 min before analyzing cells in a flow cytometer. Results are shown in FIG. 5.

Except for Piz Roseg and Piz Argient, all antibodies of the present disclosure strongly bound to CD117 on TF-1 cells. Binding could also be detected for Piz Roseg and Piz Argient, although only at higher antibody concentrations. The NegCtrl antibody (anti-chicken lysozyme) did not show any binding to TF-1 cells.

### Example 11: Inhibition of SCF-Binding in Hematopoietic Stem Cells

In this experiment it was tested if the antibodies of the present disclosure are able to inhibit binding of CD117 to SCF in HSPCs (CD34+CD38− cells) and HSC's (CD34+CD38−CD90+CD45RA− cells).

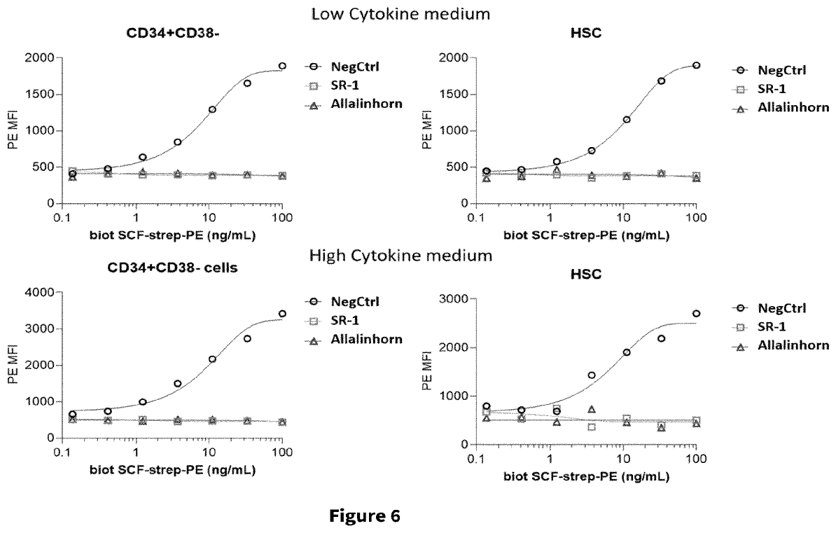

The experiment was performed in Low cytokine medium and in High cytokine medium. Low cytokine medium consist of StemPro media (Gibco), StemPro nutrients (Gibco), 50 ng/ml LDL (STEMcell Technologies), 1% Penicillin/Streptomycin (Thermo Fisher), 1% glutamine (Thermo Fisher), 20 ng/ml Flt3 (Miltenyi Biotec), 50 ng/ml TPO (Mitteny Biotec) and various concentrations of SCF (Milteny Biotec). High cytokine medium consists of the same ingredients plus 50 ng/ml IL6 (Milteny Biotec), 10 ng/ml IL3 (Milteny Biotec), 10 ng/ml IL2 (Milteny Biotec), 20 ng/ml IL7 (Milteny Biotec), 50 ng/ml IL11 (Milteny Biotec), 3 ng/ml EPO(STEMcell Technologies) and 20 ng/ml GM-CSF (Mitteny Biotec). Tested were antibodies NegCtrt, SR-1 and Allalinhorn. Results are shown in FIG. 6.

For both tested cell populations and both media the same result was achieved. Antibodies SR-1 and Allalinhorn both inhibited binding of SCF to CD117 completely and to the same extent. The NegCtrl antibody (anti-chicken lysozyme) did not inhibit binding of SCF to CD117.

### Example 12: Inhibition of Proliferation in HSPC's and HSC's

SCF-dependent proliferation was also measured in mobilized CD34-positive HSPC's isolated from peripheral blood (CliniMACS Prodigy, Mittenyi Biotech). Cells were seeded at a cell concentration of 75,000 cells/ml in sterile, white cell culture treated plates with transparent bottom in complete Stemspan AOF Media with cytokines (100 ng/ml SCF; FLT3, TPO and IL-3). Cells were treated with various concentration of the antibodies of the present disclosure. After 3 days of incubation a CellTiter-Glo 2.0 assay (Promega) was performed and luminescence was read in a luminometer (Envision, Perkin Elmer) and IC50's were determined. Results are shown in Table 22.

TABLE 22

| Antibody | IC50 [μg/ml] |
|---|---|
| NegCtrl | >10 |
| SR-1 | 0.0623 |
| AMG191 | 0.0954 |
| FSI-174 | 0.02532 |
| Dufour | 0.09488 |
| Matterhorn | 0.07156 |
| Piz Bernina | 0.00521 |
| Allalinhorn | 0.0313 |
| Piz Zupo | 0.1139 |
| Eiger | 0.5935 |
| Piz Argient | >10 |
| Piz Roseg | >10 |
| Bietschhorn | 0.08423 |
| Bellavista | 0.09599 |

The results obtained with CD34 positive HSPC's correlate nicely with the results obtained with TF-1 cells. Several of the antibodies generated showed an IC50 in this SCF-dependent proliferation assay that is in the range of, or even below that of the original SR-1 antibody, as well as the known humanized derivatives thereof.

For human CD34+ HSCs (different donors) cells are seeded in complete Stemspan AOF medium with cytokines:100 ng/ml SCF, 100 ng/ml TPO, 60 ng/ml IL-3 and 100 ng/ml FLT-3 Ligand in sterile white cell culture treated plates at a cell density of 75,000 cells/ml. HSCs are then treated with various CD117 antibodies or an isotype control antibody at different concentrations and plates are incubated for 3 days. After 3 days a CellTiter-Glo 2.0 assay (Promega) is performed and luminescence is read in a luminometer (e.g. Envision plate readers).

### Example 13: HSC Depletion Experiment

Depletion of human HSCs in mice using various mAbs was adapted from Pang et al (Blood (2019) 133:2069-78).

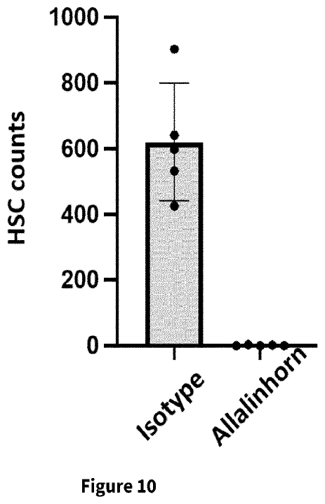

NBSGW mice (Jackson Laboratories) were injected with 1 Mio HSPCs. 8, 10, 12 and 14 days after cell injection mice received 25 mg/kg antibody Allalinhorn per dose iv. 16 weeks later the mice were euthanized and blood, spleen and bone marrow were analysed by FACS. Results for bone marrow HSC depletion are shown in FIG. 10. The figure shows in vivo depletion of HSCs after injection of Allalinhorn, compared to animals receiving an isotype control antibody. HSCs were identified by FACS as live/hCD45+/CD34+/CD38−/CD45RA−/CD90+.

In another experiment, NSG mice (Jackson Laboratories) were sub-lethally irradiated one day before injection with 1 Mio gene-edited HSPCs carrying a E73K variant of CD117. 8, 10, 12 and 14 days after cell injection mice received 25 mg/kg antibody Allalinhorn or 25 mg/kg isotope control antibody i.v. per dose. 16 weeks later the mice were euthanized and blood, spleen and bone marrow were analysed by FACS.

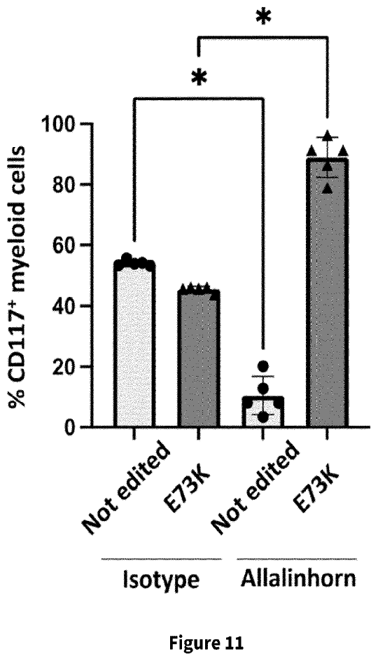

Results for bone marrow are shown in FIG. 11. CD117+ myeloid cells were identified by FACS as live/hCD45+/CD33+/CD117+. To identify CD117+ cells the antibody clone 104D2 was used, which does not interfere with binding of SR-1 and Allalinhorn to CD117. Clone 104D2 was used as an expression control. Cells were classified as not edited (104D2+ and SR-1+) or E73K edited (104D2+ but SR-1−) based on double staining with anti-CD117 clone SR-1 and anti-CD117 clone 104D2.

FIG. 11 shows depletion of unedited cells in mice receiving antibody Allaninhorn compared to mice receiving an isotype control antibody. In contrast, injection of Allaninhorn resulted in an enrichment of E73K variant cells compared to animals receiving isotype control antibody.

### Example 14: Removal of the Critical Sites in the CDRs has a Beneficial Effect on Antibody Stability

The positive effect of the removal of the critical sites is tested by subjecting the antibodies to different stress conditions, such as heat stress and pH stress.

For heat stress, the antibody solutions are incubated at 50° C., protected from light for 5 days. For the pH stress condition, the antibody solutions are diluted to 1 mg/ml in PBS and buffer exchanged against either 20 mM citrate buffer pH3 or 20 mM Tris buffer pH9. Low pH and high pH samples are then incubated at room temperature, protected from light for 1, 3 and 7 days. At the end of the incubation period, the stressed samples are neutralized by dilution 1:2 in 200 mM phosphate buffer pH7.4 and stored at −80° C. All samples are then analyzed by high-performance size exclusion chromatography (HP-SEC), Cation exchange chromatography (CEX), CE-SDS and/or RP-HPLC and peptide mapping to identify chemical and physical degradation. To evaluate the impact of the forced degradation on the biological activity of the molecule, measurement of binding to CD117 is also performed on the stressed antibodies.

### Example 15: Antibody-Mediated Inhibition of HSPC Colony Formation

The effect of the antibodies on HSPC colony formation was tested as follows. HSPCs were plated on methylcellulose (500 cells per 35 mm dish, in duplicate) containing hematopoietic cytokines (MethoCult GF H84435), and allowed to grow for 14 days at 37° C., 5% C02, in the presence of the anti-CD117 antibodies SR-1, Dufour and Allalinhorn. The antibodies were added in the medium once at the time of plating at the indicated final concentrations (0.1, 1 and 10 μg/mL). Colonies were counted manually using an inverted microscope (and a gridded scoring dish).

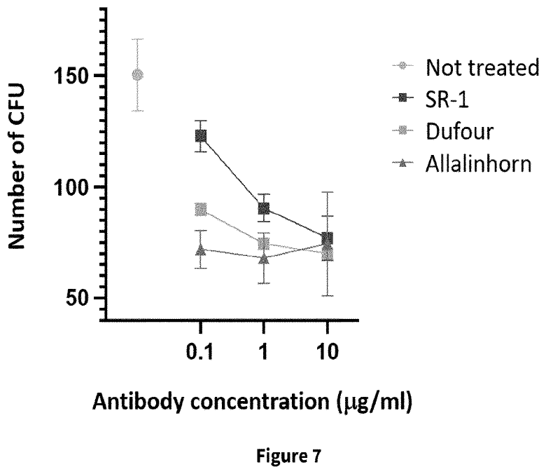

Results are shown in FIG. 7. All three anti-CD117 antibodies tested led to a dose-dependent reduction of colony. Reduction in colony formation was most pronounced with antibody Allalinhorn.

### Example 16: SCF-Dependent Phosphorylation of CD117

TF-1 cells (wild type, knock out, and variants E73K, D121K and S123K of CD117) were seeded at a concentration of 1 million cells/mL in 2 mL of Medium (RPM11640 supplemented with GlutaMAX+10% heat inactivated FBS) in a 6-well plate. Cells were treated with 1-20 ng/mL recombinant human SCF (1 mg/mL PeproGMP Recombinant Human SCF) for 5 minutes, before cell collection. Cells were washed with ice-cold PBS, before resuspending cells in 500 μL Lysis Buffer (Lysis buffer from Signaling containing Protease Inhibitor and Phosphatase Inhibitor). Cells were flash frozen in liquid nitrogen and thawed at 37° C. twice, before freezing them at −80° C. until further usage. Undiluted samples were thawed and processed according to the manufacturers protocol for the PathScan Phospho-c-Kit (Tyr719) Sandwich ELISA Kit (Cell Signaling). Absorbance (OD_450 nm) was assessed using the Envision (Perkin Elmer).

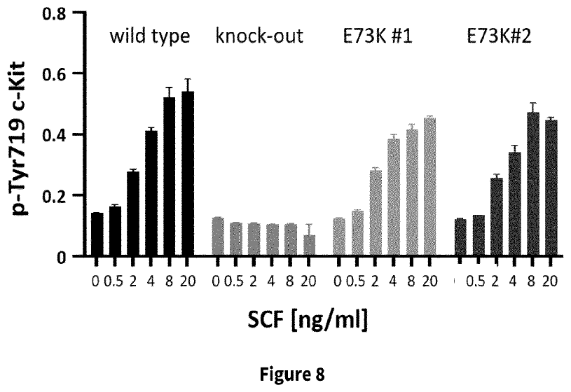

Exemplary results for variant E73K are shown on FIG. 8. Wild-type TF-1 cells and the E73K, D121K and S123K variants of CD117 showed a SCF-dependent phosphorylation at position Tyr719 of CD117. Phosphorylation levels of the knock-out cells are at the background level.

### Example 17: Preserved Signal Transduction of CD117 Variants in the Presence of Blocking Antibodies

TF-1 cells (wild type, and variants E73K, E73Y, D121K, S123F and S123K of CD117) were seeded at a concentration of 1 million cells/mL in 2 mL of Medium (RPM11640 supplemented with GlutaMAX+10% heat inactivated FBS) in a 6-well plate. Cells were pre-treated with 0.5 μg/mL of antibodies Piz Bernina or Allalinhorn for 30 minutes, before adding 100 ng/mL recombinant human SCF (1 mg/mL PeproGMP Recombinant Human SCF) for 5 minutes. Cells were collected, washed once with ice-cold PBS and resuspended in 500 μL Lysis Buffer (Lysis buffer from Signaling containing Protease Inhibitor and Phosphatase Inhibitor). Cells were then flash frozen in liquid nitrogen and thawed at 37° C. twice, before freezing them at −80° C. until further usage. Undiluted samples were thawed and processed according to the manufacturers protocol for the PathScan Phospho-c-Kit (Tyr719) Sandwich ELISA Kit (Cell Signaling) or the Human c-Kit (CD117) ELISA Kit (Abcam). Absorbance (OD_450 nm) was assessed using the Envision (Perkin Elmer).

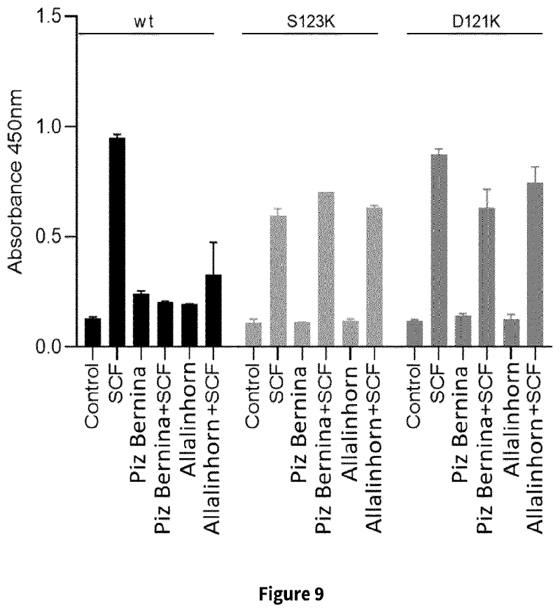

Exemplary results for variants D121K and S123K are shown in FIG. 9. SCF-dependent CD117 phosphorylation in wild-type TF-1 cells is blocked by antibodies Piz Bernina or Allalinhorn, whereas in TF-1 cells with D121K and S123K variants of CD117 phosphorylation is unaffected. The same is also observed for variants E73K, E73Y and S123F (data not shown).

### Example 18: CD117 Variants are Resistant to Treatment with ADCs

In this experiment, DF-1 cells were used. DF-1 cells lack CD117. Cells were transfected with selected CD117 variants (E73Y, D121K and S123K). Transfected cells were treated with ADCs (antibodies Piz Bernina and Allalinhorn, both coupled to teserine) at a concentration of 0.1, 1 and 10 μg/ml. After 48 hours cells were analyzed by FACS.

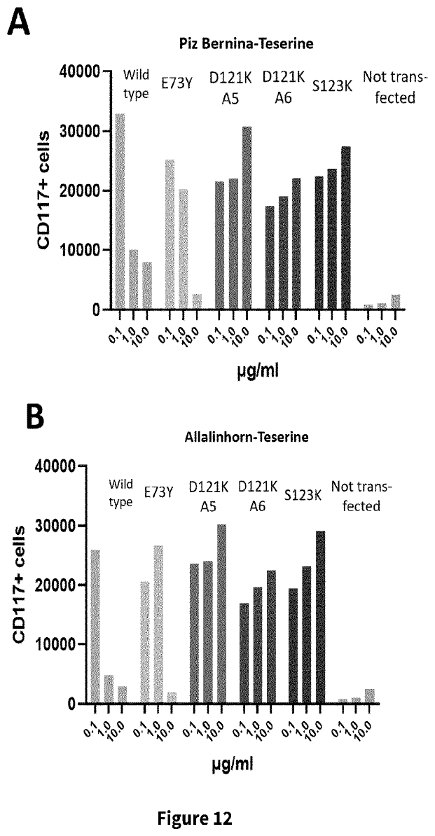

Results are shown in FIG. 12. D121K A5 and D121K A6 designate two different batches of plasmids for the same variant. Both tested ADCs, Piz Bernina-teserine and Allalinhorn-teserine, led to an effective depletion of wild-type DF-1 cells, but not DF-1 cells transfected with D121K or S123K variants of CD117.

### Example 19: Additional HSC Depletion Experiment

Depletion of human HSCs in mice using various mAbs was adapted from Pang et al (Blood (2019) 133:2069-78).

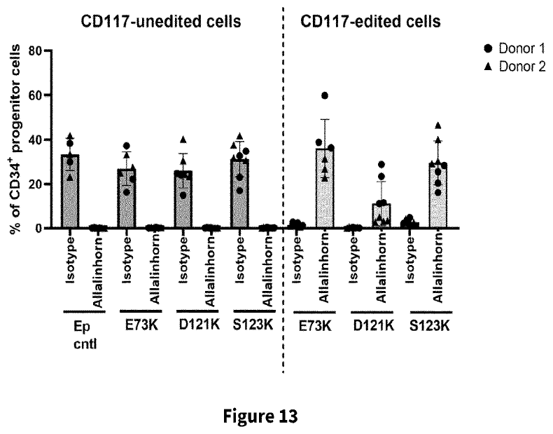

NBSGW mice (Jackson Laboratories) were injected with 1 Mio HSPCs from 2 different donors carrying a E73K, S123K or D121K variant of CD117. Control groups of mice received unedited electroporation control HSPCs. 7, 9, 11 and 12 days after cell injection mice received 4 mg/kg antibody Allalinhorn per dose i.v. 16 weeks later the mice were euthanized and blood, spleen and bone marrow were analysed by FACS. Results for depletion of CD117-unedited cells and enrichment of CD117-edited cells are shown in FIG. 13. To identify CD117+ cells the antibody clone 104D2 was used, which does not interfere with binding of SR-1 and Allalinhorn to CD117. Clone 104D2 was used as an expression control. Cells were classified as unedited (104D2+ and SR-1+) or edited (104D2+ but SR-1−) based on double staining with anti-CD117 clone SR-1 and anti-CD117 clone 104D2.

FIG. 13 shows depletion of unedited CD34+ progenitor cells (Gated as live/hCD45+/CD34+/CD38−) in mice receiving antibody Allalinhorn compared to mice receiving an isotype control antibody. In contrast, injection of Allalinhorn resulted in an enrichment of E73K, D121K and S123K-edited variant cells compared to animals receiving isotype control antibody.

1.-15. (canceled)

16. A humanized antibody or antibody fragment specific for human CD117, wherein said antibody or antibody fragment comprises a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9, wherein said humanized antibody or antibody fragment has at least a mutation in which the asparagine in the LCDR1 region (SEQ ID NO: 7) is replaced by glutamic acid or tyrosine.

17. The humanized antibody or antibody fragment according to claim 16, wherein said humanized antibody or antibody fragment has one or more of the additional following mutations:
a) the first asparagine in the HCDR2 region (SEQ ID NO: 5) is replaced by a serine, b) the second asparagine in the LCDR3 region (SEQ ID NO: 9) is replaced by lysine, or c) the aspartic acid in the LCDR3 region (SEQ ID NO: 9) is replaced by glutamic acid.

18. The humanized antibody or antibody fragment according to claim 17, wherein said humanized antibody or antibody fragment comprises
a) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 95,
b) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 81, the LCDR2 region of SEQ ID NO: 8 and the LCDR3 region of SEQ ID NO: 113,
c) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 113,
d) a variable heavy chain comprising a HCDR1 region of SEQ ID NO: 4, a HCDR2 region of SEQ ID NO: 71, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 95,
e) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 81, the LCDR2 region of SEQ ID NO: 8 and the LCDR3 region of SEQ ID NO: 95,
f) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 79, the LCDR2 region of SEQ ID NO: 8 and the LCDR3 region of SEQ ID NO: 9, or
g) a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 81, the LCDR2 region of SEQ ID NO: 8 and the LCDR3 region of SEQ ID NO: 9.

19. The humanized antibody or antibody fragment according to claim 18, wherein said humanized antibody or antibody fragment comprises:
a) a variable heavy chain of SEQ ID NO: 22 and a variable light chain of SEQ ID NO: 98,
b) a variable heavy chain of SEQ ID NO: 22 and a variable light chain of SEQ ID NO: 99,
c) a variable heavy chain of SEQ ID NO: 22 and a variable light chain of SEQ ID NO: 100,
d) a variable heavy chain of SEQ ID NO: 70 and a variable light chain of SEQ ID NO: 98,
e) a variable heavy chain of SEQ ID NO: 22 and a variable light chain of SEQ ID NO: 97,
f) a variable heavy chain of SEQ ID NO: 22 and a variable light chain of SEQ ID NO: 61, or
g) a variable heavy chain of SEQ ID NO: 22 and a variable light chain of SEQ ID NO: 62.

20. The humanized antibody according to claim 19, wherein said antibody is of the human IgG1 class, optionally wherein said antibody is a silenced IgG1 antibody, such as an antibody with a PA-LALA mutation.

21. The humanized antibody or antibody fragment according to claim 20, wherein said antibody or antibody fragment inhibits SCF binding to CD117, SCF-dependent proliferation of CD117-positive cells and/or SCF-dependent phosphorylation of CD117.

22. The humanized antibody or antibody fragment according to claim 21, wherein said antibody or antibody fragment binds to CD117 with at least the same affinity as an antibody or antibody fragment comprising a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9.

23. The humanized antibody or antibody fragment according to claim 22, wherein said antibody or antibody fragment binds to the same epitope on CD117 as an antibody or antibody fragment comprising a variable heavy chain comprising a HCDR1 region of SEQ ID NO: 4, a HCDR2 region of SEQ ID NO: 5, a HCDR3 region of SEQ ID NO: 6 and a variable light chain comprising, the LCDR1 region of SEQ ID NO: 7, the LCDR2 region of SEQ ID NO: 8 and the LCDR3 region of SEQ ID NO: 9.

24. The humanized antibody or antibody fragment according to claim 23, wherein said antibody or antibody fragment is a monoclonal antibody or antibody fragment.

25. The humanized antibody or antibody fragment according to claim 24 for use in medicine.

26. The humanized antibody or antibody fragment according to claim 25, where said use in medicine is the treatment of cancer, such as a hematological cancer or a solid cancer, or the treatment of an inflammatory diseases, an autoimmune disease, urticaria, prurigo nodularis, eosinophilic esophagitis, or mastocytosis.

27. A nucleic acid composition comprising a nucleic acid sequence or a plurality of nucleic acid sequences encoding the humanized antibody or antibody fragment according to claim 16.

28. A vector comprising the nucleic acid composition of claim 27.

29. A host cell comprising the nucleic acid composition of claim 27.

30. A pharmaceutical composition comprising the humanized antibody or antibody fragment according to claim 16 and a pharmaceutically acceptable carrier or excipient.
